(19) United States
(12) Patent Application Publication
(10) Pub. No.: US 2016/0010096 A1
(43) Pub. Date: Jan. 14, 2016

(54) HIGH LINOLENIC ACID PRODUCING BRASSICA PLANTS

(71) Applicant: CARGILL INCORPORATED, Wayzata, MN (US)

(72) Inventors: Zhizheng CHEN, Fort Collins, CO (US); Honggang ZHENG, Fort Collins, CO (US)

(21) Appl. No.: 14/694,747

(22) Filed: Apr. 23, 2015

Related U.S. Application Data
(63) Continuation of application No. PCT/US14/62732, filed on Oct. 28, 2014.
(60) Provisional application No. 61/896,528, filed on Oct. 28, 2013.

(51) Int. Cl. C12N 15/82; A01H 5/10

(52) U.S. Cl. CPC C12N 15/8247; C12N 15/8274; C12N 15/8286; A01H 5/10; C12N 15/8278; C12N 15/8275

(57) ABSTRACT

The present disclosure is directed to Brassica plants having elevated levels of linolenic acids, including non-transgenic plants having elevated levels of linolenic acids. Also described herein are methods for the production of those plants and oils derived from such plants.

### CROSS-REFERENCE TO RELATED APPLICATIONS

This application claims the benefit of priority to U.S. Provisional Patent Application No. 61/896,528 filed Oct. 28, 2013, which is incorporated herein by reference in its entirety.

This application contains a sequence listing submitted electronically via EFS-web, which serves as both the paper copy and the computer readable form (CRF) and consists of a file entitled “SequenceListing_{—}033449_{—}8087_W000.txt”, which was created on Oct. 28, 2014, which is 36,864 bytes in size, and which is herein incorporated by reference in its entirety.

### BACKGROUND

Plants and particularly plant seeds, which are often adapted to store significant amounts of lipids, represent a significant source of those compounds. Plant lipids, including seed oils, have a variety of uses including their use as animal feed, culinary shortening, flavoring, and as texturing agents for human consumption. In addition, some lipids that can be produced in plants, such as omega-3 fatty acids (e.g., linolenic acids), are believed to provide health benefits relative to saturated fats and other oils. Although plants and their seeds represent a significant source of lipids, consumer acceptance and regulatory hurdles for transgenic plants have limited the ability to produce and use plant oils with specifically tailored profiles for feed and culinary applications. The development of plants, and specifically non-transgenic plants, that can produce oils with desirable profiles, including those with increased omega-3 fatty acid content, is therefore deemed desirable.

### SUMMARY

Linolenic acid levels in Brassica napus seeds are generally in the range of 5-13%. Low linolenic mutants can have levels lower than 1%. Provided in the present disclosure are non-transgenic Brassica plants having high levels (e.g., greater than 15%, 16%, 17%, or 18%) of 18:3 fatty acids and particularly high levels of linolenic acids (e.g., alpha and/or gamma linolenic acids) derived by mutagenesis. In some embodiments, the high 18:3 fatty acid content of the seed oil fraction from the seed of those plants shows a negative correlation with 18:2 fatty acid content (R value: −0.74) and/or 18:1 fatty acid content (R-value: −0.77).

Analysis of Single Nucleotide Polymorphism (SNP) profiles of progeny of B. napus plants that have undergone mutagenesis has permitted the identification of two genomic blocks that significantly correlate with the increased 18:3 fatty acid phenotype. The candidate genes were mapped to B. napus (AACC; 2n=38) an allopolyploid species formed by the hybridization of ancestors of B. oleracea that has a type genome (CC 2n=18) and B. rapa that has a type “A” genome (AA 2n=20).

QTL (quantitative trait loci) mapping identified two genomic blocks which show significant correlation with the increased C18:3 fatty acid phenotype. The first genomic block was located on the B. napus chromosome N12 (which corresponds to the “C2” chromosome of B. oleracea). The second genomic block was located on the B. napus chromosome N17 (which corresponds to the “C7” chromosome of B. oleracea). Accordingly, the present disclosure provides for Brassica plants (e.g., B. napus, B. oleracea, B. juncea, and/or B. rapa) and parts thereof, including seed, comprising all or part of the loci associated with the chromosome N12 block flanked by SNP markers C2-p16531874 and C2-p51360247 of B. napus line rrm1367-003, and particularly plants and their seeds that display a high 18:3 fatty acid phenotype. The present disclosure also provides for Brassica plants and parts thereof, including seeds, comprising all or part of the genomic sequence associated with chromosome N17 flanked by SNP markers C7-p4690293 and C7-p22897297 of B. napus line rrm1367-003, and particularly plants and their seeds that display a high 18:3 fatty acid phenotype.

In another embodiment, the disclosure includes and provides for a Brassica plant (e.g., B. napus, oleracea, juncea, and/or rapa) having a non-transgenic low-saturated-fat trait that produces seed (or a plant cell of a seed) having an oil fraction with a linolenic acid content of at least 15, 16, 17, 18, 19, 20, 21, or 22 percent by weight. In addition to the high 18:3 fatty acid content of the oil fraction recovered from the Brassica plants or seeds described above, the oil fraction may also have a low erucic acid content.

In addition to the plants and seeds providing the high 18:3 fatty acid phenotype described above, this disclosure includes and provides for a meal fraction from those plants and/or seeds.

Embodiments of the foregoing plants, oil and/or the meal fraction produced from those plants, have a sufficiently low glucosinolate and erucic acid contents to be classified as canola varieties or products from canola varieties.

Unless otherwise defined, all technical and scientific terms used herein have the same meaning as commonly understood by one of ordinary skill in the art to which this invention belongs. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description, and from the claims.

### BRIEF DESCRIPTION OF THE DRAWINGS

FIG. 1 depicts a genetic linkage map of the chromosome N12 of B. napus showing the genomic block associated with the increased 18:3 fatty acid content in the mutant rrm1367-003. A total of 1115 SNP markers in this genomic segment giving R-values >=0.70 have been identified.

FIG. 2 depicts a genetic linkage map of the chromosome N17 of B. napus showing the genomic block associated with the increased 18:3 fatty acid content (e.g., ALA) in the mutant rrm1367-003. A total of 625 SNP markers in this genomic block (4-22.9 Mb positions) giving R-values ranging from 0.45 to 0.52 have been identified.

FIG. 3 depicts the weight percent of 18:3 fatty acids in the seed “oil fraction” of 196 F2 lines derived from a cross between B. napus mutant line rrm1367-003 and an elite breeding line RO011. Also shown are the percentages of 18:3 fatty acids in the seed oil fraction from the seed of two F2 parental lines rrm1367-003 (16.2% w/w) and RO011 (9.5% w/w), and B. napus cv. Topas (6.9% w/w), which was the line subjected to radiation mutagenesis to produce rrm1367-003.

### DETAILED DESCRIPTION

### 1.0 Definitions

Throughout this disclosure, the terms “plant” and “plants” include parts thereof unless stated otherwise. Parts of plants include, but are not limited to, any one or more of: a leaf, pollen, an ovule, an embryo, a cotyledon, a hypocotyl, a meristematic cell, callus, a microspore, a root, a root tip, a pistil, an anther, a flower, a seed, a shoot, a stem, a pod, petiole and a cell or protoplast of any thereof.

“High 18:3” trait or phenotype or “increased 18:3” trait or phenotype as used herein means plants of the Brassicaceae (e.g., B. napus, B. oleracea, B. juncea etc.) whose seeds have an oil fraction with greater than 16% of 18:3 fatty acids by weight. Embodiments of increased 18:3 fatty acid content include plants with a seed oil fraction having greater than 16%, 17%, or 18%, such as plants with an 18:3 fatty acid content in a range selected from 16-19, 16-20, 18-22, 19-23, 20-22, 21-23, or 22-24 percent.

“Low-saturated-fat trait” or “low saturated fatty acid trait” as used herein means plants of the Brassicaceae (e.g., B. napus, B. juncea) whose seeds have an oil fraction with less than 7% by weight of the fatty acids present in the oil fraction. Embodiments of reduced saturated fatty acid content include plants with a seed oil fraction having less than 6%, 5%, 4.5%, 4%, or 3.5%, such as plants with a saturated fatty acid content in a range selected from 7-5, 6-4.5, 5-3.5, or 5-2 percent.

As used herein 22:1 or C22:1 refers to fatty acids having a linear chain of 22 carbon atoms, a terminal carboxyl group that may or may not be esterified, and one double bond between carbon atoms (e.g., erucic acid C22:1 omega 9).

As used herein 18:3 or C18:3 refers to fatty acids having a linear chain of 18 carbon atoms, a terminal carboxyl group that may or may not be esterified, and three double bonds between carbon atoms (e.g., alpha linolenic acid and/or gamma linolenic acid).

As used herein 18:2 or C18:2 refers to fatty acids having a linear chain of 18 carbon atoms, a terminal carboxyl group that may or may not be esterified, and two double bonds between carbon atoms (e.g., linoleic acid).

As used herein 18:1 or C18:1 refers to fatty acids having a linear chain of 18 carbon atoms, a terminal carboxyl group that may or may not be esterified, and one double bond between carbon atoms (e.g., oleic acid).

With regard to saturated fatty acids, as used herein: 24:0 or C24:0 refers to lignoceric acid; 22:0 or C22:0 refers to behenic acid; 20:0 or C20:0 refers to arachidic acid; 18:0 or C18:0 refers to stearic acid; 16:0 or C16:0 refers to palmitic acid; and 14:0 or C14:0 refers to myristic acid, the terminal carboxyl groups of any of which may or may not be esterified unless indicated otherwise.

As used herein, total saturated fatty acid content, “total sats” or “sats” refers to the total of myristic acid (C14:0), palmitic acid (C16:0), stearic acid (C18:0), arachidic acid (C20:0), behenic acid (C22:0), and lignoceric acid (C24:0).

“Meal fraction,” “defatted meal” or “defatted meal fraction” as used herein means the solid remainder of Brassica seed after it is air dried and hexane extracted as follows. Seed is dried in ambient air by adjusting the temperature to achieve 9% moisture and flaked from0.38 to 0.64 cm in a ribbon blender. The flakes are cooked in a stack cooker at 82±1° C. for 30 min at 8.5% moisture, after which they are pre-pressed with vertical and horizontal bar spacing set to 0.031 cm, a vertical shaft speed of 40 RPM (revolutions per minute), and a horizontal shaft speed of 25 rpm to form a pressed cake of meal. The press cake is subsequently extracted in a Crown Model 2 extractor at 37.3 kg and hexane extracted with a 2:1 solvent to solids ratio and dried to remove residual hexane and form the meal fraction.

“Crush oil fraction” as used herein refers to the oil released from the pressing of Brassica seed without organic solvent extraction (e.g., hexane or isooctane extraction). After recovery from pressing, oil from the seed may be allowed to settle (e.g., at room temperature) to separate out any aqueous phase particulates, and the oil may be filtered (e.g., through a 0.2 micron filter) to remove particulate solids.

“Oil fraction” or “seed oil fraction” as used herein refer to C14 to C24 fatty acids typically extracted by isooctane from base hydrolyzed plant material, such as seeds, using the protocol set forth in Example 1. Unless stated otherwise, the percentages, changes in percent composition, or ratios of fatty acids are given as changes on a weight basis (e.g., percent by weight) based on the weight of the total C14-C24 fatty acids present in the oil fraction.

“Transgenic” as used in reference to plants or “genetically modified organisms” (GMO) as used herein are organisms (e.g., Brassica plants) whose genetic material has been altered using techniques generally known as “recombinant DNA technology.” Recombinant DNA technology is the ability to combine DNA molecules from different sources into one molecule ex vivo (e.g., in a test tube). This terminology generally does not cover organisms whose genetic composition has been altered by conventional cross-breeding or by “mutagenesis” breeding, as these methods predate the discovery of recombinant DNA techniques. See World Health Organization, Biorisk management Laboratory biosecurity guidance, 2006 World Health Organization (WHO/CDS/EPR/2006.6).

“Non-transgenic” as used herein refers to plants and food products derived from plants that are not “transgenic” or “genetically modified organisms” as defined above.

“Permissive plants” or “permissive Brassica plants” are Brassica plants (e.g., lines or varieties) that have an increase in the 18:3 content of their seed oil fraction when the chromosomal fragment between C2-p1653187 and C2-p51360247 of B. napus line rrm1367-003 and/or SNP markers C7-p4690293 and C7-p22898729 of C7 of B. napus rrm1367-003 are introduced into their genome by cross breeding.

As used herein “the same or substantially the same conditions” with reference to plant growth means two or more conditions (e.g., soil conditions, photoperiod and light intensity, soil moisture, humidity, temperature, etc.) under which a population of genetically identical plants would grow with phenotype traits that are statistically indistinguishable.

Weight percent,” “percent by weight,” or “wt %” of a fatty acid refers to the percent by weight of the fatty acids having from 14 carbon atoms (C14 fatty acids) to 24 carbon atoms (C24 fatty acids). When used in connection with a seed, the term refers to the percent by weight of the total of those fatty acids in the seed oil fraction.

### 2.0 Development of Fatty Acid Traits and their Molecular Mapping

The development of specific oil seed traits in members of the Brassicaceae can be accomplished by non-transgenic means, including ionizing radiation, UV light, and chemically induced mutagenesis. Cross breeding of plants and subsequent mapping of the DNA markers associated with the genetic traits permits the identification of the genetic basis for the traits. Mapping of genomic blocks responsible for traits also assists in the effective transfer of the traits into other members of the genus and/or species, including elite production lines with other desirable characteristics (e.g., disease resistance, herbicide tolerance, drought resistance, etc.).

To develop plants with elevated 18:3 oil content, and particularly seed oil with elevated 18:3 content, seed was subjected to mutagenesis and mutant lines were screened for improvement in the desired oil traits. In one embodiment, seeds of the B. napus line Topas were subjected to mutagenesis by exposure to gamma radiation. Although publically available, seeds of B. napus cv. Topas were deposited with American Type Culture Collection, 10801 University Blvd, Manassas, Va. 20110 (ATCC) and designated ATCC deposit PTA-120738 on Dec. 2, 2013. Non-transgenic (non-GMO) plants were selected for high 18:3 fatty acid content by using gas chromatography (GC) to analyze the composition of oil derived from plants grown from the mutated seed or their progeny. A series of lines having elevated 18:3 content, particularly elevated alpha linolenic acid, were developed. Seed from one such line, rrm1367-003, which has a high content of 18:3 oil in its seeds, was deposited with the ATCC under Accession number PTA-120636 on Oct. 11, 2013. The rrm1367-003 line was crossed with an elite variety, RO011, to create 196 individual F2 plants. RO011 is a breeder's code for the variety AV-Sapphire, which was released by Agriculture Victoria Services in association with Grains Research & Development Corporation (GRDC) and marketed by Dovuro Seeds since 2003. Analysis of those plants revealed a transgressive segregation of the 18:3 fatty acid content trait in this F2 population. In addition, there was a negative correlation between the 18:1 and 18:2 content of seed oil (R-value: −0.74) and between the 18:1 and 18:3 content of seed oil (R-value: −0.77). QTL analysis using a 60K SNP array purchased from Illumina, Inc., San Diego, Calif., in a subset of 173 individual F2 plants identified two genomic blocks that correlated with the elevated 18:3 fatty acid trait, one on chromosome N12 and one on chromosome N17.

2.1 Analysis of the QTL Associated with Brassica napus Chromosome N12

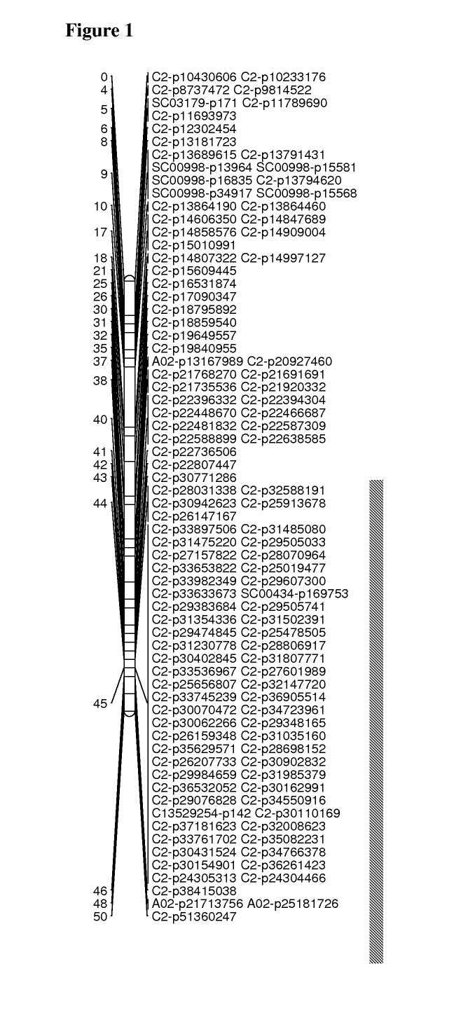

The QTL analysis indicated that the genomic block of rrm1367-003 chromosome N12 accounted for the majority of the increased 18:3 content in the seed oil fraction (R-values ranging from 0.48 to 0.74 for individual SNP markers) mapped to the region between SNP markers C2-p16531874 and C2-p51360247. The chromosomal region between SNP markers C2-p22807447 and C2-p51360247 gave a higher degree of correlation with the increased 18:3 fatty acid (R-values ranging 0.68 to 0.74 for individual SNP markers). Those chromosomal regions can be subdivided into smaller segments based on the presence of SNP markers within the region, for example as shown in Table 1 and in FIG. 1. Table 1 provides the allele types on N12 for the two crossing parental lines and physical locations for the SNP marker alleles on the C2 chromosome of B. oleracea TO1000 (B. oleracea TO1000 genome sequence version 4; released 12 Jan. 2012 from the Canseq consortium, see e.g., http://aafc-aac.usask.ca/canseq/). Table 1 also provides the locations for the SNP marker alleles on the N12 chromosome of B. napus Darmor (the Darmor genome sequence was published by Chalhoub, B. et al., in Science 345: 950-953 (2014), the B. napus Darmor genome sequence version 4.1 is available at www.genoscope.cns.fr/brassicanapus/data/).

TABLE 1
SNPs on the Chromosome N12 Genomic Block that Correlate with Elevated 18:3
Fatty Acid Content in the Seeds of B. napus Line rrm1367-003

| | | | | | Position | Position | | |
|---|---|---|---|---|---|---|---|---|
| | | | | | in B. | in B. | | |
| | | | | | oleracea | napus | | |
| | SNP | | rrm1367- | | TO1000 | Darmor | | |
| SEQ^{a} | Name | Sequence* | 003^{b} | RO011^{c} | (v4) | (v4.1) | R^{d} | cM^{e} |
| 1 | C2- | AAACAAATTTATTAACCAAATC | G | A | 16531874 | 13096295 | 0.48 | 25 |
| | p16531874 | GTAAAAGG[A/G]TTCCAGCTAG | | | | | | |
| | | AATTTTCTCTCTAGGTTTAA | | | | | | |
| 2 | C2- | TTGTTTAGTAGATCTCAAAAGC | G | A | 17090347 | 13743334 | 0.48 | 26 |
| | p17090347 | AGATCTTT[A/G]TATAGAGTTGG | | | | | | |
| | | TGAGTGGCTTAGTGGTTCC | | | | | | |
| 3 | C2- | GTTCTAGGCGAGAAAATGAGGT | G | A | 18795892 | 15582485 | 0.59 | 30 |
| | p18795892 | TTCTGCAA[A/G]CATACTTATCA | | | | | | |
| | | GAAAYAGACACAAGACTAA | | | | | | |
| 4 | C2- | TGCCATAGTTGGAGCTCTAGTT | G | A | 18859540 | 15647884 | 0.59 | 31 |
| | p18859540 | GTCCAAGA[A/G]CCCAATTTCTT | | | | | | |
| | | TTTGATATGTGATATCTTA | | | | | | |
| 5 | C2- | TTCGGTTCCGGTTATTTTGSCCA | C | A | 19649557 | 15999615 | 0.56 | 32 |
| | p19649557 | GGCCTAT[A/C]CGAAAGTATCG | | | | | | |
| | | AGCTTTTACATCAATGGAC | | | | | | |
| 6 | C2- | TTTTTCAAAAATAAATGAACCC | G | A | 19840955 | NA | 0.64 | 35 |
| | p19840955 | GAATGACA[A/G]TCTTTWTTAG | | | | | | |
| | | ACATTTTTTACCACTAGACC | | | | | | |
| 7 | A02- | GAGAAAAAGATCAAAGGATTC | A | G | NA | 19174453 | 0.56 | 37 |
| | p13167989 | GCCATCAGC[A/G]AAACTGCTTT | | | | | | |
| | | CTTCATCTTCCTCCTTGTTG | | | | | | |
| 8 | C2- | TGGAGGAACAGGCGTTCCTTAT | A | G | 20927460 | NA | 0.63 | 37 |
| | p20927460 | CAAATTTA[A/G]AAGAAGAAAA | | | | | | |
| | | AACCCTGCACTAATTCATCC | | | | | | |
| 9 | C2- | ACATGGCTAGCGATCCTTGGAC | A | G | 21691691 | 18340878 | 0.63 | 38 |
| | p21691691 | GTCTGCCT[A/G]CAAGAGACAT | | | | | | |
| | | GCTCTCCTCTTGGGGCTTGT | | | | | | |
| 10 | C2- | TCGGATTTGTTAATTTAGTAATT | A | G | 21735536 | 18409741 | 0.63 | 38 |
| | p21735536 | TACATCC[A/G]GTAAACATTTTC | | | | | | |
| | | ASAGCTRGTTGACTAAAA | | | | | | |
| 11 | C2- | CTCTACATAACTGATGCTGCAG | C | A | 21768270 | NA | 0.63 | 38 |
| | p21768270 | AGCAAGTA[A/C]ATGCCAAAAA | | | | | | |
| | | AATCRTTTTGAAAAAAATAG | | | | | | |
| 12 | C2- | ATATGCTGTTCTCGCCGTTTTTG | G | A | 21920332 | 18566854 | 0.63 | 38 |
| | p21920332 | TATAACT[A/G]GATTTGGGAAG | | | | | | |
| | | TTGACAGGATCATTCGGTT | | | | | | |
| 13 | C2- | AAGATCCGGCTCAATATGTCAA | A | C | 22394304 | 19161100 | 0.65 | 40 |
| | p22394304 | CCAAACTC[A/C]TATCTTACCAG | | | | | | |
| | | CAATCTTTTACACAACATC | | | | | | |
| 14 | C2- | AAACCGGACTGGAAGATGAAC | G | A | 22396332 | 19167990 | 0.65 | 40 |
| | p22396332 | CATCTACTT[A/G]TTTGGWTCAT | | | | | | |
| | | GGTTCAATATGGTTCAACTG | | | | | | |
| 15 | C2- | TCAATTGTGCCGTTGCAGCTTG | A | G | 22448670 | 19212172 | 0.65 | 40 |
| | p22448670 | CATTGTAT[A/G]TTTTAAAAGTT | | | | | | |
| | | AATTAACACGTCGACCTTA | | | | | | |
| 16 | C2- | TAAAATAAATTGCAGGGTACAA | G | A | 22466687 | NA | 0.65 | 40 |
| | p22466687 | CCAGGGGA[A/G]GGCGCACGGT | | | | | | |
| | | GTAATGTACGGGGGCGCGTA | | | | | | |
| 17 | C2- | CTTATAAAGTTATAACTAGATG | G | A | 22481832 | 19255063 | 0.65 | 40 |
| | p22481832 | TTTTGTTC[A/G]TATATGCATGT | | | | | | |
| | | ATAATTCTTTTATGATAMT | | | | | | |
| 18 | C2- | RAGATACAAAATTTGCTCAACG | G | A | 22587309 | 19578237 | 0.65 | 40 |
| | p22587309 | TTTTAAAA[A/G]GAACATTAAT | | | | | | |
| | | ATAGGCCTAATCCAACAAAG | | | | | | |
| 19 | C2- | TAACCTGCAARAAAAAAACAA | C | A | 22588899 | 19597612 | 0.65 | 40 |
| | p22588899 | AGACTTAAG[A/C]TTTGGAATC | | | | | | |
| | | GTCAAGAAGCAATGAAAACAT | | | | | | |
| 20 | C2- | AATTTTTTTCTAATACTGTATCT | A | C | 22638585 | 19646220 | 0.65 | 40 |
| | p22638585 | CAAACCA[A/C]GTATAGAAAAT | | | | | | |
| | | AGTAAATTATATAAGAGTT | | | | | | |
| 21 | C2- | TGTTTTCGTCGTTGAACTTTGTG | A | G | 22736506 | 19817882 | 0.65 | 41 |
| | p22736506 | AATCAAT[A/G]ATGACTTTTAAA | | | | | | |
| | | TTCTTATAAGTGTGGAGG | | | | | | |
| 22 | C2- | CAACACAATCGTGCGAGTTTTT | G | A | 22807447 | 19905502 | 0.66 | 42 |
| | p22807447 | TTATTTCT[A/G]TACTTAAATAT | | | | | | |
| | | TTTAGTTTATATTCCCTCA | | | | | | |
| 23 | C2- | TGGCTTGTTAGGACATGTAAAT | A | G | 24304466 | 21248508 | 0.69 | 45 |
| | p24304466 | AAGAATGA[A/G]GTTAATAAAC | | | | | | |
| | | ATGGGTAGTTCGTAGAAGAA | | | | | | |
| 24 | C2- | GTGTACAAAAGCTAGAATGAAT | G | A | 24305313 | 21249359 | 0.68 | 45 |
| | p24305313 | TTTTATAA[A/G]AACACAGAGA | | | | | | |
| | | ACATGTTSAACAAAATTTCA | | | | | | |
| 25 | C2- | AAGACCGTTCCATTTCCATACC | C | A | 25019477 | NA | 0.71 | 45 |
| | p25019477 | ATGGTTTA[A/C]TTCTCTCATSC | | | | | | |
| | | AATATATGMTTAATGTAAG | | | | | | |
| 26 | C2- | TAGATAGCTAGTTATATATAAA | A | G | 25478505 | NA | 0.71 | 45 |
| | p25478505 | CAAGAGTC[A/G]TTACACTAAG | | | | | | |
| | | TATTACACTATACCTTAATC | | | | | | |
| 27 | C2- | ATTAAACAAGAATGAAAGTACT | A | G | 25656807 | 22112830 | 0.71 | 45 |
| | p25656807 | GTTTTGCA[A/G]TGAAAGAAAA | | | | | | |
| | | GCYCTTCTTGTCTGTTTATT | | | | | | |
| 28 | C2- | TTTAGAAAATCCGGAAATGGAC | A | G | 25913678 | NA | 0.71 | 44 |
| | p25913678 | TATTCATC[A/G]GTATCAATTCG | | | | | | |
| | | TGGGAATTTATTTGAGTTT | | | | | | |
| 29 | C2- | ATCCTAAACAAAAGAAATCATT | G | A | 26147167 | 22537784 | 0.71 | 44 |
| | p26147167 | ATAATAAT[A/G]TCGAATAAAA | | | | | | |
| | | ATACCTAAAGACTAAGATTA | | | | | | |
| 30 | C2- | AAGACCGGATATMGGTCAGGG | A | G | 26159348 | 22551394 | 0.71 | 45 |
| | p26159348 | TCAGGGCCG[A/G]CCCAGAGGG | | | | | | |
| | | AAAACCACCAAGCCAACGATT | | | | | | |
| 31 | C2- | CTATATTATTAAAATAGAAAAA | A | G | 26207733 | 22593499 | 0.71 | 45 |
| | p26207733 | CAAATATA[A/G]ATTTACCCTAA | | | | | | |
| | | AATTTATAAATTATTTACA | | | | | | |
| 32 | C2- | GGAGAGACTYCGGAGTTYTTCA | A | C | 27157822 | 20046680 | 0.72 | 45 |
| | p27157822 | GATCTAAA[A/C]GAAGTTTACG | | | | | | |
| | | ACAGGAACCGGCACCGATTG | | | | | | |
| 33 | C2- | AGATGGATATTGTCTAGTAAAT | C | A | 27601989 | 11895115 | 0.71 | 45 |
| | p27601989 | CTAGTATA[A/C]AAATTATGGTG | | | | | | |
| | | AGCTAGGTAGTATACCTTG | | | | | | |
| 34 | C2- | TGTGGATTATAGATGACTTCAA | G | A | 28031338 | 25649343 | 0.71 | 44 |
| | p28031338 | TCCGTGTT[A/G]TCAAAGRAAA | | | | | | |
| | | AARAAAGATGACTTSAATCC | | | | | | |
| 35 | C2- | AAAACGAAGGTTTTACCCTGAA | A | G | 28070964 | 25685604 | 0.71 | 45 |
| | p28070964 | AATGCTGC[A/G]GGAAGCCTTG | | | | | | |
| | | CTGATACCCCGCACCTTGTG | | | | | | |
| 36 | C2- | KAATSTACAAGMAGTGATAGA | G | A | 28698152 | 26319446 | 0.71 | 45 |
| | p28698152 | CTAGTGATW[A/G]ACATCTTGC | | | | | | |
| | | AATGATGTGGGAAGAGGTTCA | | | | | | |
| 37 | C2- | TCAATGGAAGGCMRAAAAGAA | A | G | 28806917 | 26423543 | 0.71 | 45 |
| | p28806917 | TCRTAATCC[A/G]AGAACCCCTA | | | | | | |
| | | TCAGGTCCTGACCACGAGAT | | | | | | |
| 38 | C2- | GATATCACCAAGAATCGGAGA | G | A | 29076828 | 25030200 | 0.71 | 45 |
| | p29076828 | GAATCAGTG[A/G]AAACAAGGA | | | | | | |
| | | AAATGATGAATCAGAAAGTAA | | | | | | |
| 39 | C2- | GTTGGTTCAAAAGTAGTCGTCC | C | A | 29348165 | 24763406 | 0.71 | 45 |
| | p29348165 | ACACGGAC[A/C]ATGCTGCATT | | | | | | |
| | | AAAATATTTGATGCAAAAGA | | | | | | |
| 40 | C2- | CCAGACGGCTTTCGCTTAGCCA | C | A | 29383684 | NA | 0.71 | 45 |
| | p29383684 | GCTCTGTT[A/C]TGCACCTCGCA | | | | | | |
| | | ATGCARTCCATAATGCTAG | | | | | | |
| 41 | SC00434- | AGAACAATCTGATCGCCCGCGA | A | C | NA | 30033659 | 0.71 | 45 |
| | p169753 | CTCGACCA[A/C]CCAAAAGATA | | | | | | |
| | | CAGACGACCCCCCAAATTGA | | | | | | |
| 42 | C2- | TTTCGCTGTTCAATCTCATCAG | G | A | 29474845 | 24653698 | 0.71 | 45 |
| | p29474845 | AAACCTGC[A/G]ACAAAAATCC | | | | | | |
| | | YGATACCAGGGGTCTGATTG | | | | | | |
| 43 | C2- | CGATTAAAGGTTACATACATAC | C | A | 29505033 | NA | 0.71 | 45 |
| | p29505033 | CTTTTGGG[A/C]CGCTTATAATT | | | | | | |
| | | TAGGAAGGGCTTYGACRAA | | | | | | |
| 44 | C2- | TGTTTTTGAATTATTAGATTGG | C | A | 29505741 | NA | 0.71 | 45 |
| | p29505741 | AATTTGCA[A/C]TCTCTAAGAAT | | | | | | |
| | | TTTATTTAGGCAAATGATA | | | | | | |
| 45 | C2- | TGCCACTACCAACTCCACCAAA | C | A | 29607300 | 24563136 | 0.71 | 45 |
| | p29607300 | GTCCTTTC[A/C]MGCATCACATA | | | | | | |
| | | GATCGGACTCAATCTCAAG | | | | | | |
| 46 | C2- | ACCCCAGCAACATCATGAAGG | A | C | 29984659 | 24167548 | 0.72 | 45 |
| | p29984659 | GCAGCCTTC[A/C]ACTGCTRCAA | | | | | | |
| | | CTTAATGAAGCATACATACT | | | | | | |
| 47 | C2- | TTTGACATCAGCTCGACCTCAT | G | A | 30062266 | 24094189 | 0.71 | 45 |
| | p30062266 | TCTTAAAC[A/G]CTATCATTCTT | | | | | | |
| | | TTGCCCGACTTTGTTGACA | | | | | | |
| 48 | C2- | AGAATGGAACAACTACTGACTC | G | A | 30070472 | 24077275 | 0.71 | 45 |
| | p30070472 | CGCAATTC[A/G]CAGAAACGAC | | | | | | |
| | | ATGGTGCTGAAAAAAATAGG | | | | | | |
| 49 | C2- | ATCCAATTTGAAAAAAAAAATC | A | G | 30110169 | 24017753 | 0.72 | 45 |
| | p30110169 | TTCAGCAA[A/G]TGATAGGTTG | | | | | | |
| | | ACACATGGCGACAAATGCCC | | | | | | |
| 50 | C2- | AATGAACTCATCATTGTAGATG | A | G | 30154901 | 23970397 | 0.70 | 45 |
| | p30154901 | GTGTTCTT[A/G]ACCCAAGCAAT | | | | | | |
| | | AAKATGAAGTTTGCAACCA | | | | | | |
| 51 | C2- | CCTTTAGGATATGTGTGAATAT | A | C | 30162991 | 23965797 | 0.71 | 45 |
| | p30162991 | TAAGGGTC[A/C]ACGACTTACA | | | | | | |
| | | AGAGAAAAAAGGTAAAAGTT | | | | | | |
| 52 | C2- | CTTGTCGGGAGCTTGATTCTTG | A | G | 30402845 | 23341322 | 0.71 | 45 |
| | p30402845 | TCTCGGAG[A/G]ACAAGATTCT | | | | | | |
| | | CGGCTTCGAGAGCCCCGAGC | | | | | | |
| 53 | C2- | TTTGGTGAGAAAGATATATTGT | A | C | 30431524 | 23386148 | 0.71 | 45 |
| | p30431524 | ACGCACAT[A/C]ATTCGTTGATG | | | | | | |
| | | TAATATTTTATAGTTGTAA | | | | | | |
| 54 | C2- | AGGGAGCGATTTAGGGCTTTTA | A | C | 30771286 | 23698972 | 0.71 | 43 |
| | p30771286 | TCAGATCT[A/C]TTTTATACACA | | | | | | |
| | | AGAAGAAAAACATTTCCAC | | | | | | |
| 55 | C2- | ATGCTGGCATTATGAAAATGTT | A | C | 30902832 | 26508525 | 0.72 | 45 |
| | p30902832 | TGTAACAC[A/C]CGTATTTTTTK | | | | | | |
| | | AAATAACTTAAATAAATAA | | | | | | |
| 56 | C2- | CGCACGTAGTGTCTACTCCTTG | A | G | 30942623 | 26548494 | 0.72 | 44 |
| | p30942623 | ACACCAAC[A/G]AGAGATTCTA | | | | | | |
| | | TCYATTGTGGATAACCTGTT | | | | | | |
| 57 | C2- | GTATATTAGATATTGTGAAATA | C | A | 31035160 | 26681745 | 0.71 | 45 |
| | p31035160 | TGGTTCTA[A/C]MTAAGCTAAA | | | | | | |
| | | TCTCATGTATATCGTATCTT | | | | | | |
| 58 | C2- | AAATCTTTCCACTGATTTTCCAT | C | A | 31230778 | 26887633 | 0.71 | 45 |
| | p31230778 | CAATGGT[A/C]TAAACTCATGC | | | | | | |
| | | ATCTTAGCTATCACATTTG | | | | | | |
| 59 | C2- | ATTGATTRGGCCGCCTAATTGG | A | G | 31354336 | 27031653 | 0.71 | 45 |
| | p31354336 | TTAAATGG[A/G]CCGCTTGATTG | | | | | | |
| | | GCGTCCGATTTTGTTTTGC | | | | | | |
| 60 | C2- | GAAGCTGTAAATTTYCTTGGGA | A | C | 31475220 | NA | 0.71 | 45 |
| | p31475220 | TGACCGAG[A/C]GCATTGGTAT | | | | | | |
| | | CGTCCGACAAAGCATGAGTT | | | | | | |
| 61 | C2- | ATAAGATGTGCTTTTGTAAGAT | A | C | 31485080 | NA | 0.71 | 45 |
| | p31485080 | AATATTAA[A/C]TTTTGAWTTTT | | | | | | |
| | | GGCTTTATTATTTTGTTAA | | | | | | |
| 62 | C2- | TCAATATTAGGTATTGCAAAAA | A | C | 31502391 | 26186376 | 0.71 | 45 |
| | p31502391 | CTCCTAGC[A/C]AATTAACMGG | | | | | | |
| | | TTTATCTCACTTGACTAATT | | | | | | |
| 63 | C2- | TATTGGATAGCTCAGAATTGAT | G | A | 31807771 | 27504352 | 0.71 | 45 |
| | p31807771 | TAAAGCCA[A/G]AAGAAGAGAA | | | | | | |
| | | GGAAGTATTGGGGCCAWGYA | | | | | | |
| 64 | C2- | GTAATTTTAGATCTGAAAATCT | C | A | 31985379 | NA | 0.71 | 45 |
| | p31985379 | GAATTCGT[A/C]GAAAATGTAG | | | | | | |
| | | AAATAGTTTTAAAATTGAAA | | | | | | |
| 65 | C2- | ATGCTTGCCAATTTTATTAATA | G | A | 32008623 | NA | 0.71 | 45 |
| | p32008623 | CTATATAC[A/G]ATTTTAAAATA | | | | | | |
| | | ATTCAATACGTACATTCTT | | | | | | |
| 66 | C2- | TAGCTAACGTTTAAGCAAATTT | G | A | 32147720 | NA | 0.71 | 45 |
| | p32147720 | TTGTCAAC[A/G]AAAAATATTCC | | | | | | |
| | | TTGGAGGTCAAAATCTAGT | | | | | | |
| 67 | C2- | GTCATTCACTAATAACACATGT | A | G | 32588191 | NA | 0.71 | 44 |
| | p32588191 | TTATTGTA[A/G]AAGAAGAAAG | | | | | | |
| | | AAACGACGAAGAAMAAAAAG | | | | | | |
| 68 | C2- | ATTATCACTAGAATACCACAAC | G | A | 33536967 | 29212900 | 0.72 | 45 |
| | p33536967 | TTGGTTAC[A/G]ATATGGATCTT | | | | | | |
| | | GAGTTTGCTTCTTTACTKC | | | | | | |
| 69 | C2- | TGTTTTCTTGTAGTGACAGTAC | C | A | 33633673 | 29272119 | 0.72 | 45 |
| | p33633673 | CGTGTTAC[A/C]GCCCTACCAAA | | | | | | |
| | | CCGGAAGGAAACTCCAAGA | | | | | | |
| 70 | C2- | TTTCGAAGAGAGCCCACGAAW | C | A | 33653822 | NA | 0.71 | 45 |
| | p33653822 | CAAACAAAC[A/C]AAAAAAGCT | | | | | | |
| | | CAAATAAAAAGATGCCACATG | | | | | | |
| 71 | C2- | ACACTCAGGTCGACATTAATGT | G | A | 33745239 | 29402272 | 0.71 | 45 |
| | p33745239 | CATCTCGA[A/G]TTCGGGATGG | | | | | | |
| | | CCCTTCTTCTTTCSTAGACC | | | | | | |
| 72 | C2- | GGTTTCATGTTCAGAACCAACT | C | A | 33761702 | 29418709 | 0.71 | 45 |
| | p33761702 | GTAACATG[A/C]GAGAAAATAT | | | | | | |
| | | TTGATATAGAAAGACAAAAC | | | | | | |
| 73 | C2- | TGGTTCCCACAGTAAAGGAGAT | G | A | 33897506 | NA | 0.71 | 45 |
| | p33897506 | CATGCAGG[A/G]TCCTCGCCGG | | | | | | |
| | | AGATCATGCTGTCGTTACAR | | | | | | |
| 74 | C2- | CAATATTTTATAAGGGCCATAA | C | A | 33982349 | 29605688 | 0.71 | 45 |
| | p33982349 | AACTTCGR[A/C]GCCAGCTTGTA | | | | | | |
| | | ATTAGATCTCATCACAGCT | | | | | | |
| 75 | C2- | TAGCTATAAATCTAGAAACCTG | A | G | 34550916 | 31121006 | 0.71 | 45 |
| | p34550916 | TAGAAACT[A/G]AGTTTTACAG | | | | | | |
| | | ATTTACTGTATTCTACGGAT | | | | | | |
| 76 | C13529254- | GCAAAAACTTCCAAATTTTGAA | G | A | NA | 30205470 | 0.74 | 45 |
| | p142 | TGAATAGT[A/G]GGAGACTGAA | | | | | | |
| | | TRTTGATTCCACATTTCCAT | | | | | | |
| 77 | C2- | TTGTGCCTTTAAGTTTTCGGGA | A | G | 34723961 | 30189229 | 0.71 | 45 |
| | p34723961 | ATTTTTCA[A/G]ATATAACCTAT | | | | | | |
| | | ATATTCATTTGAAACATAA | | | | | | |
| 78 | C2- | AAAGAAGACATYAATAACAAA | A | G | 34766378 | 30233970 | 0.71 | 45 |
| | p34766378 | AAAAACTAT[A/G]TAAGGAAGC | | | | | | |
| | | GAGGACTGAAGAGAAAGCGAA | | | | | | |
| 79 | C2- | ATCCGGGACATCGAATATTCGA | G | A | 35082231 | NA | 0.71 | 45 |
| | p35082231 | GTGGCTAC[A/G]GATCGAATCG | | | | | | |
| | | GATCGGATAATTGATTATTC | | | | | | |
| 80 | C2- | TGGATGATACTTGTCAAACGTG | C | A | 35629571 | 28165080 | 0.71 | 45 |
| | p35629571 | TGGAGAAG[A/C]GGTCGAAACG | | | | | | |
| | | CTGAACCATATGCTGTTTCA | | | | | | |
| 81 | C2- | ATAAAACCGCAGTGATAACAA | A | C | 36261423 | 31427281 | 0.71 | 45 |
| | p36261423 | CTTTAATTC[A/C]AAAAGCTCCA | | | | | | |
| | | AATGACAGAGATATGGATGG | | | | | | |
| 82 | C2- | TACTAAAATTCAATTGGTTAAA | G | A | 36532052 | 31681457 | 0.71 | 45 |
| | p36532052 | AATTGTGG[A/G]AAATAGTCAA | | | | | | |
| | | TTGCATAAACTAATGCATTG | | | | | | |
| 83 | C2- | CTGAAATTCTATGTAATCTAAT | C | A | 36905514 | NA | 0.71 | 45 |
| | p36905514 | GGACTGAT[A/C]TGAGATACTCT | | | | | | |
| | | GTATGTTGGTGGTCTTTRT | | | | | | |
| 84 | C2- | CATAAGCTTAGCTTGTGTGTAC | A | G | 37181623 | 32175061 | 0.71 | 45 |
| | p37181623 | TTAAGAAA[A/G]AACTCAAGAA | | | | | | |
| | | ATCAATAGTTTATAGTTTAT | | | | | | |
| 85 | C2- | TGAAGAAATCTACATGGAGCA | G | A | 38415038 | 33117513 | 0.71 | 46 |
| | p38415038 | GCCAGAGGG[A/G]TTCATTGTT | | | | | | |
| | | AAAGGCAAGGAAGACTGGGTG | | | | | | |
| 86 | A02- | ATCAAGAAAATTACAAATTCAC | G | A | NA | 40255672 | 0.71 | 48 |
| | p21713756 | GTAGATGA[A/G]ATATACAGTA | | | | | | |
| | | GGTTAAACATATAACAACCA | | | | | | |
| 87 | A02- | ATCAGTATATACACTCACGGTT | C | A | NA | 44041755 | 0.73 | 48 |
| | p25181726 | CCGCGAGC[A/C]GSTCTTTTTGC | | | | | | |
| | | CTCAGGTTTATTGGCTGAC | | | | | | |
| 88 | C2- | GAAATGTTGCGAATATATGCTT | G | A | 51360247 | 44957822 | 0.65 | 50 |
| | p51360247 | AAAAGTAA[A/G]GTTATATTTCT | | | | | | |
| | | AATATGCAGTTCGAAATAG | | | | | | |

^{a}SEQ = SEQ ID NO.
^{b}rrm1367-003 = Nucleotide appearing in mutant line rrm1367-003
^{c}RO011 = Nucleotide appearing in the elite B. napus line RO011
^{d}R = Correlation Coefficient
^{e}cM = centiMorgan(s)
*the variable SNP base is shown in brackets
NA = Position Not Available

Comparative genomic analysis employing the genome sequence of B. oleracea TO1000 indicates the presence of candidate genes contributing to the seed oil traits of rrm1367-003 may be present in the chromosome N12 genomic block between SNP markers C2-p16531874 and C2-p51360247. Those candidate genes include FAB1 encoding Fatty acid biosynthesis 1, LPAT4 encoding Lysophosphatidyl acyltransferase 4, LRD2 and LACS2, encoding a long chain acyl-CoA, KCS20 encoding fatty acid elongase, mtACP3 encoding mitochondrial acyl carrier protein 3, KCS21, encoding a member of the 3-ketoacyl-CoA synthase family, and ACBP5 encoding acyl-CoA binding protein 5. For example, among the candidate genes, the KCS20 gene is recognized to be involved in the biosynthesis of VLCFA (Very Long Chain Fatty Acids). KCS20 is located between the two flanking SNP markers C2-p37254117 and C2-p37285344, which corresponds to the gene sequence present at position 37264817 to37267297 on the C2 chromosome of B. oleracea TO1000. The locations of potential candidate genes including KCS20 and their flanking SNP markers are shown in Table 2a. In addition to the candidate genes shown in Table 2a, in one embodiment, the disclosure includes the chromosomal region between the locations corresponding to nucleotides 51360247 and 52859203 on the C2 chromosome of Brassica oleracea, TO1000, which comprises a gene for an acyl-CoA N-acyltransferases (NAT) superfamily protein.

TABLE 2a

| | | | | | Position in | |
|---|---|---|---|---|---|---|
| | | | | | B. oleracea | |
| | SNP | | rrm1367- | | TO1000 | |
| SEQ^{a} | Name | Sequence* | 003^{b} | RO011^{c} | (v.4) | R-value |
| 89 | C2-p23082339 | AACAGAATGGCACCAGGTGTGT | A | G | 23082339 | 0.66 |
| | | TGGATCTC[A/G]CACTATAGGAA | | | | |
| | | GCACGAAAGATCTGTRTCT | | | | |
| | FAB1, KAS2 | | | | 23114001- | |
| | (AT1G74960) | | | | 23116673 | |
| | LPAT4 | | | | 23122707- | |
| | (AT1G75020) | | | | 23124889 | |
| 90 | C2-p23898427 | AGGTTGAATTTGATCGTTATAA | A | G | 23898427 | 0.67 |
| | | AAGATCTC[A/G]TGTGCAAGTTT | | | | |
| | | AACAACATCACCATCGTTM | | | | |
| 91 | C2-p32635329 | GAAAGAACAGTTGGCCGAAGC | A | G | 32635329 | 0.72 |
| | | ATTGACTAA[A/G]CCACTAGAAC | | | | |
| | | GATCAAGAGTTCAGGAGCTT | | | | |
| | LRD2 & LACS2 | | | | 32638678- | |
| | (AT1G49430) | | | | 32642533 | |
| 92 | C2-p32643944 | TGTACTTCTTATAGCTTTAACAA | A | G | 32643944 | 0.71 |
| | | CCGATCC[A/G]GACAGTACAAT | | | | |
| | | GCAAAGTGACCTAGTTTGG | | | | |
| 93 | C2-p37254117 | GGAGATTCCAATTAAGGCCAAA | G | A | 37254117 | 0.71 |
| | | ACTGAGTC[A/G]GTAGAATGGTT | | | | |
| | | CTTAGCTCAATTAGTGGAG | | | | |
| | KCS20 | | G | A | 37264817- | 0.71 |
| | (AT5G43760) | | | | 37267297 | |
| 94 | C2-p37285344 | AACTCTAAATATGCAATCCGAA | A | G | 37285344 | 0.71 |
| | | GAATTAAA[A/G]CAGGAGATGG | | | | |
| | | AAGGGTTCTCACAGACCTGA | | | | |
| 95 | C2-p41012763 | GCCCAAATATCATAAAGAAACA | A | G | 41012763 | 0.68 |
| | | CACGAAAC[A/G]CTCTCCGTTTT | | | | |
| | | TGGTTTTGATCCCAGACAG | | | | |
| | mtACP3 | | A | G | 41493634- | 0.68 |
| | (AT5G47630) | | | | 41494099 | |
| | KCS21 | | | | 47537495- | |
| | (AT5G49070) | | | | 47538868 | |
| | ACBP5 | | | | 48440166- | |
| | (AT5G27630) | | | | 48444662 | |
| 88 | C2-p51360247 | GAAATGTTGCGAATATATGCTT | G | A | 51360247 | 0.68 |
| | | AAAAGTAA[A/G]GTTATATTTCT | | | | |
| | | AATATGCAGTTCGAAATAG | | | | |

^{a}SEQ = SEQ ID NO.
^{b}rrm1367-003 = Nucleotide appearing in mutant line rrm1367-003
^{c}RO011 = Nucleotide appearing in the elite B. napus line RO011
*the variable SNP base is shown in brackets

Comparative genomic analysis employing the genome sequence of B. napus Darmor indicates the presence of candidate genes that may contribute to the seed oil traits of rrm1367-003 may be present in the chromosome N12 genomic block of rrm1367-003 between SNP markers C2-p16531874 and C2-p51360247. A number of candidate genes that are involved in acyl lipid metabolism are listed in Table 2b.

TABLE 2b

| | Start of | End of | | | |
|---|---|---|---|---|---|
| Darmor | gene in | gene in | | | |
| Gene | Darmor | Darmor | Arabidopsis | | |
| Name^{a} | (V4.1) | (4.1) | Locus ID^{b} | GO Term^{b} | Description |
| BnaC02g17470D | 13064655 | 13066707 | AT1G67730 | GO: 0042761 | YBR159, KCR1; ketoreductase/ |
| | | | | | oxidoreductase |
| BnaC02g17730D | 13334685 | 13336079 | AT1G67980 | GO: 0042409 | CCoAMT; caffeoyl-CoA O- |
| | | | | | methyltransferase |
| BnaC02g18330D | 14345144 | 14345548 | AT1G68530 | GO: 0042335 | CUT1, POP1, CER6, G2; KCS6 (3- |
| | | | | | KETOACYL-COA SYNTHASE 6); |
| | | | | | catalytic/transferase, transferring acyl |
| | | | | | groups other than amino-acyl groups |
| BnaC02g18340D | 14361373 | 14362326 | AT1G68530 | GO: 0042335 | CUT1, POP1, CER6, G2; KCS6 (3- |
| | | | | | KETOACYL-COA SYNTHASE 6); |
| | | | | | catalytic/transferase, transferring acyl |
| | | | | | groups other than amino-acyl groups |
| BnaC02g20100D | 16436367 | 16438007 | AT1G70670 | GO: 0008150 | caleosin-related family protein |
| BnaC02g20500D | 17059284 | 17065032 | AT1G71010 | GO: 0044267 | phosphatidylinositol-4-phosphate 5- |
| | | | | | kinase family protein |
| BnaC02g20600D | 17124380 | 17125729 | AT1G71160 | GO: 0042335 | KCS7 (3-KETOACYL-COA |
| | | | | | SYNTHASE 7); acyltransferase/ |
| | | | | | catalytic/transferase, transferring acyl |
| | | | | | groups other than amino-acyl groups |
| BnaC02g20910D | 17552199 | 17559219 | AT1G71960 | GO: 0042626 | ABC transporter family protein |
| BnaC02g21040D | 17657806 | 17660824 | AT1G72110 | GO: 0008150 | O-acyltransferase (WSD1-like) family |
| | | | | | protein |
| BnaC02g21440D | 18352055 | 18355703 | AT1G72520 | GO: 0040007 | lipoxygenase, putative |
| BnaC02g21680D | 18609969 | 18613056 | AT1G72970 | GO: 0010430 | EDA17; HTH (HOTHEAD); FAD |
| | | | | | binding/aldehyde-lyase/mandelonitrile |
| | | | | | lyase |
| BnaC02g21760D | 18685218 | 18687137 | AT1G73050 | GO: 0046202 | (R)-mandelonitrile lyase, putative/(R)- |
| | | | | | oxynitrilase, putative |
| BnaC02g22260D | 19102583 | 19103285 | AT1G73550 | GO: 0012505 | lipid binding |
| BnaC02g22330D | 19173927 | 19177091 | AT1G73680 | GO: 0051707 | pathogen-responsive alpha-dioxygenase, |
| | | | | | putative |
| BnaC02g22460D | 19512763 | 19513059 | AT1G73780 | GO: 0008289 | protease inhibitor/seed storage/lipid |
| | | | | | transfer protein (LTP) family protein |
| BnaC02g24210D | 21509791 | 21511678 | AT1G76680 | GO: 0031407 | OPR1; 12-oxophytodienoate reductase |
| BnaC02g24660D | 21843883 | 21847833 | AT1G77740 | GO: 0003006 | 1-phosphatidylinositol-4-phosphate 5- |
| | | | | | kinase, putative/PIP kinase, putative/ |
| | | | | | PtdIns(4)P-5-kinase, putative/ |
| | | | | | diphosphoinositide kinase, putative |
| BnaC02g25190D | 22562373 | 22564354 | AT1G78690 | GO: 0008152 | phospholipid/glycerol acyltransferase |
| | | | | | family protein |
| BnaC02g27480D | 25391900 | 25395416 | AT4G00520 | GO: 0006637 | acyl-CoA thioesterase family protein |
| BnaC02g28920D | 28320133 | 28324178 | AT4G11030 | GO: 0006633 | long-chain-fatty-acid--CoA ligase, |
| | | | | | putative/long-chain acyl-CoA |
| | | | | | synthetase, putative |
| BnaC02g30500D | 32274605 | 32277354 | AT5G43760 | GO: 0009922 | KCS20 (3-KETOACYL-COA |
| | | | | | SYNTHASE 20); fatty acid elongase |
| BnaC02g30940D | 32836208 | 32842294 | AT5G44240 | GO: 0015662 | haloacid dehalogenase-like hydrolase |
| | | | | | family protein |
| BnaC02g30970D | 32849245 | 32849973 | AT5G44240 | GO: 0015662 | haloacid dehalogenase-like hydrolase |
| | | | | | family protein |
| BnaC02g32230D | 34714155 | 34716442 | AT5G46290 | GO: 0004312 | KAS I (3-KETOACYL-ACYL |
| | | | | | CARRIER PROTEIN SYNTHASE I); |
| | | | | | catalytic/fatty-acid synthase/ |
| | | | | | transferase, transferring acyl groups |
| | | | | | other than amino-acyl groups |
| BnaC02g33390D | 35848230 | 35850773 | AT5G47630 | GO: 0005575 | mtACP3 (mitochondrial acyl carrier |
| | | | | | protein 3); acyl carrier/cofactor binding |
| BnaC02g33480D | 35897951 | 35900472 | AT5G47730 | GO: 0006810 | SEC14 cytosolic factor, putative/ |
| | | | | | polyphosphoinositide-binding protein, |
| | | | | | putative |
| BnaC02g33900D | 36218888 | 36222814 | AT2G01320 | GO: 0042626 | ABC transporter family protein |
| BnaC02g35520D | 38220631 | 38223911 | AT3G25585 | GO: 0016780 | ATAAPT2; AAPT2 |
| | | | | | (AMINOALCOHOLPHOSPHOTRANSFERASE); |
| | | | | | phosphatidyltransferase/ |
| | | | | | phosphotransferase, for other substituted |
| | | | | | phosphate groups |
| BnaC02g35530D | 38245451 | 38250737 | AT3G25610 | GO: 0015662 | haloacid dehalogenase-like hydrolase |
| | | | | | family protein |
| BnaC02g35560D | 38297830 | 38301105 | AT3G25620 | GO: 0042626 | ABC transporter family protein |
| BnaC02g36350D | 39419430 | 39421700 | AT3G26790 | GO: 0010373 | FUS3 (FUSCA 3); DNA binding/ |
| | | | | | transcription activator/transcription |
| | | | | | factor |
| BnaC02g37030D | 40002669 | 40003899 | AT3G27660 | GO: 0010344 | OLE3; OLEO4 (OLEOSIN 4) |
| BnaC02g37590D | 40546448 | 40548252 | AT3G28910 | GO: 0042761 | ATMYB30; MYB30 (MYB DOMAIN |
| | | | | | PROTEIN 30); DNA binding/ |
| | | | | | transcription factor |
| BnaC02g38440D | 41445298 | 41451930 | AT5G48230 | GO: 0009793 | EMB1276; ACAT2 (ACETOACETYL- |
| | | | | | COA THIOLASE 2); acetyl-CoA C- |
| | | | | | acetyltransferase/catalytic |
| BnaC02g38450D | 41451986 | 41453468 | AT5G48230 | GO: 0009793 | EMB1276; ACAT2 (ACETOACETYL- |
| | | | | | COA THIOLASE 2); acetyl-CoA C- |
| | | | | | acetyltransferase/catalytic |
| BnaC02g38640D | 41539189 | 41539500 | AT5G48485 | GO: 0009627 | DIR1 (DEFECTIVE IN INDUCED |
| | | | | | RESISTANCE 1); lipid binding/lipid |
| | | | | | transporter |
| BnaC02g38800D | 41783404 | 41787189 | AT5G48880 | GO: 0019395 | PKT1, KAT5; PKT2 (PEROXISOMAL |
| | | | | | 3-KETO-ACYL-COA THIOLASE 2); |
| | | | | | acetyl-CoA C-acyltransferase/catalytic |
| BnaC02g38930D | 41869878 | 41871251 | AT5G49070 | GO: 0012505 | KCS21 (3-KETOACYL-COA |
| | | | | | SYNTHASE 21); acyltransferase/ |
| | | | | | catalytic/transferase, transferring acyl |
| | | | | | groups other than amino-acyl groups |
| BnaC02g39080D | 42089507 | 42093682 | AT5G49460 | GO: 0006085 | ACLB-2 (ATP CITRATE LYASE |
| | | | | | SUBUNIT B 2); ATP citrate synthase |
| BnaC02g39790D | 42724221 | 42729031 | AT5G27630 | GO: 0006869 | ACBP5 (ACYL-COA BINDING |
| | | | | | PROTEIN 5); acyl-CoA binding |
| BnaC02g40590D | 43675108 | 43679061 | AT5G25370 | GO: 0046466 | PLDALPHA3 (PHOSPHLIPASE D |
| | | | | | ALPHA 3); phospholipase D |
| BnaC02g41520D | 44381595 | 44384530 | AT5G23940 | GO: 0048730 | EMB3009 (embryo defective 3009); |
| | | | | | transferase/transferase, transferring acyl |
| | | | | | groups other than amino-acyl groups |
| BnaC02g41620D | 44429529 | 44434000 | AT5G23450 | GO: 0030148 | ATLCBK1 (A. THALIANA LONG- |
| | | | | | CHAIN BASE (LCB) KINASE 1); D- |
| | | | | | erythro-sphingosine kinase/ |
| | | | | | diacylglycerol kinase |
| BnaC02g41740D | 44509475 | 44514537 | AT5G23190 | GO: 0010345 | CYP86B1; electron carrier/heme |
| | | | | | binding/iron ion binding/ |
| | | | | | monooxygenase/oxygen binding |
| BnaC02g42690D | 45192282 | 45196242 | AT5G63770 | GO: 0048366 | ATDGK2 (Diacylglycerol kinase 2); |
| | | | | | diacylglycerol kinase |

^{a}Darmor gene name from www.genoscope.cns.fr/brassicanapus/data/
^{b}Arabidopsis Locus Name and GO Term are available at www.arabidopsis.org.

Overall fatty acid synthesis and its regulation may be more complicated in plants than in any other organism. How plants control the very different amounts and types of lipids produced in different tissues and the transcriptional regulation of enzymes involved in fatty acid biosynthesis and oil accumulation in plants remain largely unknown. Without being bound by any theory, one possible mode for the high C18:3 phenotype observed in the rrm1367-003 line may be: (1) boosted expression of FAD3 by an unknown mechanism, such Fad3 gene duplication or enhanced Fad3 gene expression; (2) an increased rate of C18:2 and/or C18:3 transportation into the desired locations; and/or (3) blocked elongation of C18 fatty acids.

2.2 Analysis of the QTL Associated with Brassica napus Chromosome N17

The second genomic block identified in the QTL analysis as correlating with the phenotypic increase in 18:3 fatty acid content in the seed oil fraction, is located on chromosome N17. That genomic block maps to a location between SNP markers C7-4690293 and C7-P22897297 of B. napus line rrm1367-003 (R-values ranging from 0.46 to 0.52 for individual SNP markers). That chromosomal region can be subdivided into smaller segments based upon the presence of SNP markers within the region, for example as shown in Table 3. Table 3 provides the physical locations for the SNP marker alleles on the C7 chromosome of B. oleracea TO1000 (B. oleracea TO1000 genome sequence version 4; released 12 Jan. 2012 from Canseq consortium http://aafc-aac.usask.ca/canseq/). Table 3 also provides the locations for the SNP marker alleles on the N17 chromosome of B. napus Darmor (the Darmor genome sequence was published by Chalhoub, B. et al., in Science 345: 950-953 (2014), and the B. napus Darmor genome sequence, version 4.1, is available at www.genoscope.cns.fr/brassicanapus/data/).

TABLE 3
SNPs on Chromosome N17 Genomic Block that Correlates with Elevated 18:3
Fatty Acid Content in the Seeds of B. napus Line rrm1367-003

| | | | | | Position | Position in | | |
|---|---|---|---|---|---|---|---|---|
| | | | | | in B. | B. napus | | |
| | SNP | | rrm1367- | | oleracea | Darmor | | |
| SEQ^{a} | Name | SEQUENCE* | 003^{b} | RO011^{c} | TO1000 (v4) | (v4.1) | R^{d} | cM^{e} |
| 96 | C7-p4690293 | AGTGATGATCCAAAACAATTAT | G | A | 4690293 | 10872310 | 0.49 | 10 |
| | | AAAAATTA[A/G]ATGGCAGCTTC | | | | | | |
| | | TCAGAAGAACTAAGCTAAC | | | | | | |
| 97 | C7-p5039845 | AAAAATCCAATTAACAATATAA | A | C | 5039845 | 10571632 | 0.50 | 10 |
| | | ATGTCTGG[A/C]TAATATTTCAG | | | | | | |
| | | TTAGCSRACCACTAACTTA | | | | | | |
| 98 | C7-p5194981 | GGCATTTGATTGAGAGAGATGA | A | G | 5194981 | NA | 0.49 | 10 |
| | | GCTTCATC[A/G]CTGATGAGAAC | | | | | | |
| | | CGAGTATGTCGTTGAAGTC | | | | | | |
| 99 | C7-p7498659 | ATTATAATACTTCTTTTGTCTTC | C | A | 7498659 | NA | 0.49 | 10 |
| | | CTACCAC[A/C]TTTATTATTTCTT | | | | | | |
| | | CCATTGAAAAGCATGCC | | | | | | |
| 100 | C7-p8599974 | AGGAACAAAATGGAAGTTGAAG | A | G | 8599974 | 482318 | 0.47 | 10 |
| | | AACACAAA[A/G]ATGGGAAGAA | | | | | | |
| | | AAAGAGAATCTAAGATTAAT | | | | | | |
| 101 | C7-p8719053 | GGGGAGATTTACACCTACCTAA | C | A | 8719053 | 7567622 | 0.49 | 10 |
| | | AGGGTCTG[A/C]AACCTCTAGTT | | | | | | |
| | | CCATGGGMAAAAGTTGTGT | | | | | | |
| 102 | C7-p8726636 | AAGTAGGTGAGCCATTGGACAT | A | G | 8726636 | 7559982 | 0.48 | 10 |
| | | GGTGAATA[A/G]GAGAGAGAGG | | | | | | |
| | | AGAAGAAACAGAGGATGAWG | | | | | | |
| 103 | C7-p8726743 | TTGACGATTCCACATGAGGTAG | G | A | 8726743 | 7559875 | 0.48 | 10 |
| | | TTGGTAGA[A/G]GTCAGCTTCTT | | | | | | |
| | | GAYGTTGGACATGTTCACA | | | | | | |
| 104 | C7-p8727745 | ACTGGTTTGCCTATAACACATAC | A | G | 8727745 | 7558873 | 0.49 | 10 |
| | | TGGTTCT[A/G]CACTTCTTCCTAC | | | | | | |
| | | TCCTCATCGTTMTCTTA | | | | | | |
| 105 | C7-p8766230 | GAACACAAATGTTGGGTCCGGA | A | G | 8766230 | NA | 0.50 | 10 |
| | | GCTGCGGG[A/G]GGCTTAGATTT | | | | | | |
| | | ACATCTCGGTTGTGTAGTT | | | | | | |
| 106 | C7-p8824122 | AAGGAATATAACTGTCTAAATA | A | C | 8824122 | NA | 0.49 | 10 |
| | | GAATATAT[A/C]ACTAATGATAC | | | | | | |
| | | AGTTATTCCTACCAGTGTA | | | | | | |
| 107 | C7-p8854349 | ACTCACAATGCCTGTATGGAAA | T | A | 8854349 | 7037418 | 0.49 | 10 |
| | | CTAAAGAA[A/T]GAGTTTCAGTT | | | | | | |
| | | GTGTTGTTCTTTCAGTATC | | | | | | |
| 108 | C7-p8870860 | GATCGAGAACATCCGCTTCTTG | G | A | 8870860 | 7020325 | 0.50 | 10 |
| | | CCTGATCM[A/G]GAGTTCCTTTG | | | | | | |
| | | CGCCGATCTGCGAGCAATG | | | | | | |
| 109 | C7-p9307503 | AGTTCATTGYCCCCTTCATGACT | A | T | 9307503 | NA | 0.48 | 10 |
| | | CGATAAA[A/T]CTCTTTTAGAAA | | | | | | |
| | | ACATTATTAGAAAACATT | | | | | | |
| 110 | C7-p9358459 | AGAAATCTGTTTTKCTTKATTKG | G | A | 9358459 | NA | 0.47 | 10 |
| | | CAAACRA[A/G]GCTAGAAAGGG | | | | | | |
| | | AGATTTAAGAAGTTGGTGA | | | | | | |
| 111 | C7-p9593996 | GGCTTTGCCCAGCTCATTACTCT | C | A | 9593996 | 590889 | 0.49 | 11 |
| | | TGCAAAA[A/C]CCCTTAATGAGA | | | | | | |
| | | GTATTATAGGTAACGATA | | | | | | |
| 112 | C7-p10040604 | ATTCAGATCACCCGATTAMAAA | A | C | 10040604 | 6002634 | 0.49 | 10 |
| | | GAGAGACG[A/C]GAGCAATAAG | | | | | | |
| | | AGAGATAAAGAGATATCAAG | | | | | | |
| 113 | C7-p10165832 | TCATTTAAGATGTTATTAACTTG | A | C | 10165832 | 5746517 | 0.49 | 10 |
| | | ACATTTT[A/C]AAATTTAAGATA | | | | | | |
| | | TCTATCTAATATTTTTTT | | | | | | |
| 114 | C7-p10180076 | AATTTATAAAATAACAGCAAAT | A | C | 10180076 | 5732917 | 0.49 | 10 |
| | | CTTAAAAA[A/C]TTTAATATRAA | | | | | | |
| | | AAATRAATTATAGATATAM | | | | | | |
| 115 | C7-p10180716 | TCAAAGTCATCTTGAAATCGAG | A | G | 10180716 | 5732277 | 0.51 | 11 |
| | | GATTCACC[A/G]GCGTAGAACTC | | | | | | |
| | | GTCGACATCGTCGCGTTGC | | | | | | |
| 116 | C7-p10212158 | TCGGGTCCTGTCCGGTGCTGTGC | C | A | 10212158 | 5688112 | 0.49 | 10 |
| | | CTAAATC[A/C]AAAGGACACCG | | | | | | |
| | | AGGAACAACACCGTACACC | | | | | | |
| 117 | C7-p10215060 | GCCATCATTGTCAAGTTCGTCGT | G | A | 10215060 | 5685224 | 0.49 | 10 |
| | | CCTATCC[A/G]TAAGAATMATW | | | | | | |
| | | GACTGGATCCTCGTCGTTT | | | | | | |
| 118 | C7-p10215325 | CGTGAGCGAGATGATTGATTCC | G | A | 10215325 | 5684959 | 0.49 | 10 |
| | | CCCACGTC[A/G]AAGTTTTCTGT | | | | | | |
| | | AGTTGTTCGAACGTAGCAG | | | | | | |
| 119 | C7-p10228536 | AAATTTATATTGGTCATTTTTWT | A | G | 10228536 | 5672363 | 0.49 | 10 |
| | | CCTATGT[A/G]CTATTTTTATGAT | | | | | | |
| | | AATTTTTTTTAGGGTTA | | | | | | |
| 120 | C7-p10261396 | TCATCCACTTCCTCCACAGCTTT | A | G | 10261396 | 5639400 | 0.49 | 10 |
| | | CTTCTCC[A/G]CAACTTCCTTCTC | | | | | | |
| | | AACCTGCTCGGCTGCTG | | | | | | |
| 121 | C7-p10262047 | GTAATGCCTAAAGAAAATCATA | A | C | 10262047 | 5638751 | 0.48 | 10 |
| | | TATAAAAT[A/C]ATGTTTCTTTG | | | | | | |
| | | ATCAAAAAGAAAAAAATCA | | | | | | |
| 122 | C7-p10613314 | TAAAATCTCCAAAATTTATTGCT | A | G | 10613314 | NA | 0.50 | 10 |
| | | CTAGCTA[A/G]GCTACAAAGTGT | | | | | | |
| | | TGTGGCCAAACATTGATG | | | | | | |
| 123 | C7-p10617039 | ATAACCAGAAACATAGCCAGAC | A | G | 10617039 | NA | 0.49 | 10 |
| | | AATGAAAG[A/G]ATATGTGATCT | | | | | | |
| | | TTCTCSAAGATCAAGTGTA | | | | | | |
| 124 | C7-p10720977 | CTTGTTTTTTCGTTAATTGGTGT | G | A | 10720977 | NA | 0.50 | 10 |
| | | AATATTT[A/G]TGTTTCATATAA | | | | | | |
| | | TTTTATTTTCAGTATTTT | | | | | | |
| 125 | C7-p11706153 | AAATAYCTCTAAAAAGTCAGAT | C | A | 11706153 | 602215 | 0.48 | 10 |
| | | ATTCGATT[A/C]TTGGCCACCCC | | | | | | |
| | | TAGGTACCTTATCAAAGCT | | | | | | |
| 126 | C7-p11718201 | TCTCTTTGATTATTTTTAACCAT | A | G | 11718201 | NA | 0.49 | 10 |
| | | ATTGACT[A/G]TAGAAACTAAAA | | | | | | |
| | | TACACATGTCAGTTTCAR | | | | | | |
| 127 | C7-p12072579 | TCTGTTATATGGTTTGAGAATTT | C | A | 12072579 | NA | 0.49 | 10 |
| | | TCAAAAT[A/C]TACTATTATTGG | | | | | | |
| | | TCTAAGAACTTATAAAAT | | | | | | |
| 128 | C7-p12079142 | CACRTGACTATATATGTGACCG | G | A | 12079142 | NA | 0.49 | 10 |
| | | RAAAAAAA[A/G]TGAAAATCTTT | | | | | | |
| | | TTTAAAAAAGGATTGATCA | | | | | | |
| 129 | C7-p12123100 | TCATGTTTAWTAATGTTTCTAGC | A | G | 12123100 | NA | 0.50 | 10 |
| | | TAATCCG[A/G]GAAGACATTAAG | | | | | | |
| | | AATTTTTCTGAGAGAGGA | | | | | | |
| 130 | C7-p12123399 | AATAAGTCAATATTTAGAAGGG | C | A | 12123399 | NA | 0.49 | 10 |
| | | GATACTTC[A/C]AGAGAAGTTTG | | | | | | |
| | | YTGTAGAGTAGACTTCCCT | | | | | | |
| 131 | C7-p12268682 | AGAGACATGGTGGAGGAGAAA | C | A | 12268682 | NA | 0.50 | 10 |
| | | ATCATCTTT[A/C]AAACATAAAA | | | | | | |
| | | AACTTTAAAATATACARAAT | | | | | | |
| 132 | C7-p12281546 | AGTTTAATTCATTTTGAGTGAAA | A | G | 12281546 | NA | 0.49 | 10 |
| | | AATGAGG[A/G]TAATCATTGGAA | | | | | | |
| | | ATGCTCTAATGTAAGCTA | | | | | | |
| 133 | C7-p12300699 | TTAAAAGTAGTCAAAATACTCA | A | G | 12300699 | 657673 | 0.50 | 10 |
| | | AAACACTT[A/G]AAATATCCAAA | | | | | | |
| | | ATATTTACGGATTCTCTAY | | | | | | |
| 134 | C7-p12301957 | ATTCCCATCATCTAACAGTTTTT | A | C | 12301957 | 656414 | 0.50 | 10 |
| | | TCAACAG[A/C]AACRACATATAA | | | | | | |
| | | ATGCTCATATAATATTTT | | | | | | |
| 135 | C7-p12356302 | GATTTATTAGATCTCATATTTTA | G | A | 12356302 | NA | 0.49 | 10 |
| | | GCCATAC[A/G]GCTCACTGGCTT | | | | | | |
| | | CTGCRTTTCATTCCCCTC | | | | | | |
| 136 | C7-p12356455 | AATTTTCATAAGACTATAGGTA | C | A | 12356455 | NA | 0.49 | 10 |
| | | ACATTATA[A/C]GAAACATTCAT | | | | | | |
| | | TCGTATAACTGAAACATCA | | | | | | |
| 137 | C7-p12385657 | TATTGTTTTTTATAAAATATTAC | A | C | 12385657 | NA | 0.50 | 10 |
| | | ATTGAGT[A/C]GGAGTTTCAATG | | | | | | |
| | | ACTTACGAAGATTAACAA | | | | | | |
| 138 | C7-p12387173 | TTAAAGGAAAAATATAATGTAT | C | A | 12387173 | NA | 0.48 | 10 |
| | | AATCCACA[A/C]GTAGCGAAGA | | | | | | |
| | | CCAAAAACCTAATATAGTGA | | | | | | |
| 139 | C7-p12401233 | AGAGGAGGGGAGCAACACTAG | G | A | 12401233 | NA | 0.49 | 10 |
| | | CAAATGGTC[A/G]TTGGTTTTGC | | | | | | |
| | | CAGCAAAAATAGGGAGATTT | | | | | | |
| 140 | C7-p12485308 | CTAGTGCTTCCCAGACCGTGTA | A | G | 12485308 | NA | 0.51 | 10 |
| | | AGAGTATG[A/G]AMATGCAAAA | | | | | | |
| | | AACAAGTGATCTCTGGTTTC | | | | | | |
| 141 | C7-p12508706 | AATTGTAACAAAGCTAAGGGTC | G | A | 12508706 | NA | 0.49 | 10 |
| | | TGATTGTT[A/G]CGGATTTTCGC | | | | | | |
| | | TTTAKGCTTTCGCTTTAGK | | | | | | |
| 142 | C7-p12512146 | AGTTAGGTTATTTGCTAGTGTAT | C | A | 12512146 | NA | 0.49 | 10 |
| | | CAACGAA[A/C]MATTTAATCCA | | | | | | |
| | | ATAGAACCTAGGATACTCA | | | | | | |
| 143 | C7-p12514520 | AAATATAGGAGATTGGAAATCC | A | G | 12514520 | NA | 0.49 | 10 |
| | | TTCTGAAC[A/G]TGTTTTTCTTGT | | | | | | |
| | | GCCATTTGATCTCATACC | | | | | | |
| 144 | C7-p12565005 | CAAATTCAGAACAATACAGACT | A | G | 12565005 | 5338696 | 0.49 | 10 |
| | | AAGCTAGC[A/G]GTGCCAAGATT | | | | | | |
| | | AAACYGTTACAATGTATAT | | | | | | |
| 145 | C7-p12684624 | TATTATAACTTGGCTAAGTTATG | A | C | 12684624 | 11305257 | 0.49 | 10 |
| | | ATCTTAC[A/C]AATTTAGCAGTG | | | | | | |
| | | TGGGTATATGCTTCTCTC | | | | | | |
| 146 | C7-p12757060 | TTGATATCTATCGGCCAAATATG | G | A | 12757060 | 11340722 | 0.49 | 10 |
| | | CTTTCAG[A/G]CCATCATATATG | | | | | | |
| | | ATTATTAATGGCATCAAG | | | | | | |
| 147 | C7-p12984513 | AGGGTATAGGAGTCCAGCATAT | C | A | 12984513 | 11602069 | 0.49 | 10 |
| | | TTGGTGTG[A/C]ATTAGCATCAA | | | | | | |
| | | TATGGTGACTTAGTATGGC | | | | | | |
| 148 | C7-p12990275 | TTTAGTTTGCATGTAAAATGTCA | G | A | 12990275 | NA | 0.50 | 10 |
| | | AACTGAC[A/G]CTCTTTGTGCMA | | | | | | |
| | | YAGAGAKAATTTAATACG | | | | | | |
| 149 | C7-p12995305 | CACATCGCACGGACTAGAATAC | G | A | 12995305 | NA | 0.50 | 10 |
| | | AGAGGACG[A/G]GGAGGTCAAG | | | | | | |
| | | GCAGAGCCGGCCYGTATACT | | | | | | |
| 150 | C7-p12995377 | TGCAACTGCAACTCTTTAGCTTT | A | G | 12995377 | NA | 0.50 | 10 |
| | | TGTTGAA[A/G]GTTTAAAGGTTT | | | | | | |
| | | TTCTCGAAATCATTTTTT | | | | | | |
| 151 | C7-p13029440 | CTGGAAAAGCATCTATAACAAT | A | C | 13029440 | NA | 0.52 | 10 |
| | | GTCATGAA[A/C]CATCTCATGAT | | | | | | |
| | | CTACYATYTGCTCTTCATG | | | | | | |
| 152 | C7-p13029555 | TTAAAATTACCATTACTACTAAT | A | G | 13029555 | NA | 0.51 | 10 |
| | | AAGCTTC[A/G]TTCACACCRATA | | | | | | |
| | | ACCTTYTCMATGTTGAAA | | | | | | |
| 153 | C7-p13069990 | CATAGAGTGAAGACTTGGCCAC | A | G | 13069990 | 13406568 | 0.50 | 10 |
| | | ATTTATTC[A/G]GAGCSCAGACA | | | | | | |
| | | TACAAGCAAACCGGTTCAT | | | | | | |
| 154 | C7-p13070860 | TGGCGCTGGCACCACCTAATCC | G | A | 13070860 | 13407432 | 0.49 | 10 |
| | | TGAAAGCG[A/G]AACTGATATAT | | | | | | |
| | | TTTTGTGGAAACATGGGGA | | | | | | |
| 155 | C7-p13083371 | CAATTTTTGTTGATSGGCAAAAA | C | A | 13083371 | 13419349 | 0.49 | 10 |
| | | AACTTGC[A/C]GAATTCCGTAAT | | | | | | |
| | | GTTACCAATCGGGCCTAA | | | | | | |
| 156 | C7-p13135120 | CGGGAAATATATTACATCTTATC | G | A | 13135120 | 13523464 | 0.49 | 10 |
| | | TATCAAA[A/G]CAGAAGTCATGA | | | | | | |
| | | TTTTTTTATGTGTGTTAT | | | | | | |
| 157 | C7-p22861548 | ACCTCGTGAGGACGATCTAACA | A | G | 22861548 | 20741654 | 0.46 | 14 |
| | | TAAAATCT[A/G]TGAACTGGTRA | | | | | | |
| | | ATCTCTCAGGACTATGTTC | | | | | | |
| 158 | C7-p22870500 | TTTTAAGTTGTTGTTTTCTTATGT | A | G | 22870500 | 20749142 | 0.46 | 14 |
| | | CTTTTT[A/G]TCCKATTCTTTTTT | | | | | | |
| | | TGTTCATATTGAATGT | | | | | | |
| 159 | C7-p22897297 | GTATGACTATTTTGTATGATTTT | G | A | 22897297 | 20769957 | 0.46 | 14 |
| | | TTCTTGA[A/G]TTCTTGTACAAG | | | | | | |
| | | ACCTGCTTTTCGACCCTG | | | | | | |

^{a}SEQ = SEQ ID NO.
^{b}rrm1367-003 = Nucleotide appearing in mutant line rrm1367-003
^{c}RO011 = Nucleotide appearing in the elite B. napus line RO011
^{d}R = Correlation Coefficient
^{e}cM = centiMorgan(s)
*the variable SNP base is shown in brackets
NA = Position Not Available

### 2.3 Development of Plants with Mutations on Chromosome N12 and Chromosome N17 Associated with Elevated Seed Oil 18:3 Content

Non-transgenic members of the Brassicaceae bearing variations in the chromosome N12 and/or chromosome N17 genomic sequences that can confer an increased ability to make and/or accumulate 18:3 fatty acids can be prepared by mutagenesis or by cross breeding of plants having variations in those genomic regions (e.g., rrm1367-003). Transgressive segregation of the C18:3 fatty acid content was observed in this F2 population compared to the C18:3 levels in the two crossing parental lines, rrm1367-003 and RO011. Of 196 F2 plants analyzed, one individual plant gave 20.57% of C18:3 fatty acid content. Accordingly, the embodiments of the present disclosure include B. napus, B. oleracea, B. rapa, or B. juncea plants or parts thereof, including cells and/or seeds, having modifications in the chromosome N12 and/or chromosome N17 genomic sequences that can cause an increase in the 18:3 fatty acid content of the plant's seed oil (e.g., when introduced into a plant line such as B. napus cv. Topas). Such plants may also have a reduction in the 18:1 content of their seed oil relative to plants that do not bear modifications in the chromosome N12 and/or chromosome N17 regions described herein, but are otherwise genetically the same or substantially the same (e.g., of the same line or variety).

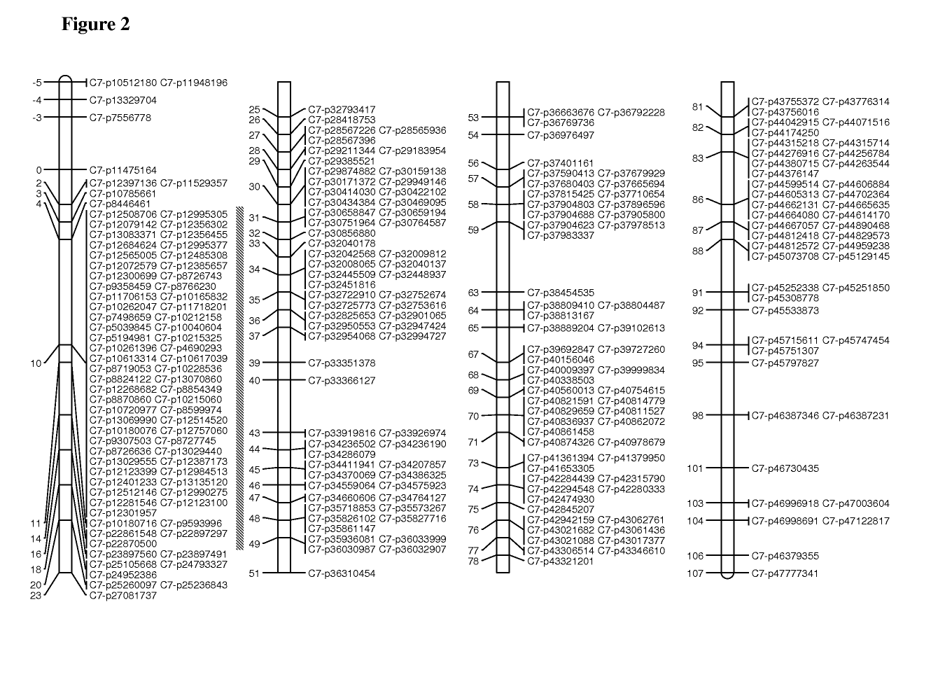

In some embodiments, B. napus, B. oleracea, B. rapa, or B. juncea plants, or parts thereof including cells and/or seeds, comprise the genomic sequence of chromosome N12 between SNP markers C2-p16531874 and C2-p51360247 or more narrowly between markers C2-p22807447 and C2-p51360247 of B. napus line rrm1367-003. In other embodiments, the plants or parts thereof may comprise any one or more segments of chromosome N12 and/or chromosome N17 of rrm1367-003 found in FIG. 1, Table 1, Table 2, FIG. 2, and/or Table 3 that give rise to an increase in 18:3 fatty acids (e.g., alpha and/or gamma linolenic acid) in the seed oil fraction when the sequence variation(s) in those regions are introduced into permissive plants such as the “Topas” cultivar. In such embodiments, the plants or parts thereof may comprise one, two, three or more sequences of chromosome N12 and/or chromosome N17 of rrm1367-003 found in FIG. 1, Table 1, Table 2, FIG. 2 and/or Table 3 that have an independently selected length greater than 10, 25, 50, 100, 200, 300, 400, 500, 1,000, 1,500, 2,000, 3,000, 5,000, 7,500, 10,000, 20,000, 30,000, 50,000, 100,000, 500,000, or 1,000,000 base pairs, or an independently selected length in a range selected from 25-50, 25-100, 50-200, 100-500, 250-1,000, 500-5,000, 2,000-10,000, 5,000-20,000, 10,000-100,000, 50,000-400,000, or 200,000-1,000,000 base pairs. Those sequences may be limited to sequences giving rise to an increase in 18:3 fatty acids (e.g., alpha and/or gamma linolenic acid) in the seed oil fraction when those regions are introduced into permissive plants such as the “Topas” variety (e.g., by cross breeding). In other embodiments, the plants or parts thereof may comprise one, two, three or more sequences of chromosome N12 and/or chromosome N17 of rrm1367-003 found in FIG. 1, Table 1, Table 2, FIG. 2 and/or Table 3 that have an independently selected length greater than 10, 25, 50, 100, 200, 300, 400, 500, 1,000, 1,500, 2,000, 3,000, 5,000, 7,500, 10,000, 20,000, 30,000, 50,000, 100,000, 500,000, or 1,000,000 base pairs, or an independently selected length in a range selected from 25-50, 25-100, 50-200, 100-500, 250-1,000, 500-5,000, 2,000-10,000, 5,000-20,000, 10,000-100,000, 50,000-400,000, or 200,000-1,000,000 base pairs that are transcribed and/or translated, and which give rise to an increase in 18:3 fatty acids (e.g., alpha and/or gamma linolenic acid) in the seed oil fraction when those regions are expressed in permissive plants such as the “Topas” cultivar.

In addition to providing non-transgenic Brassica plants having high levels of 18:3 fatty acids in the seed oil fraction, and particularly high levels of linolenic acids (e.g., alpha and/or gamma linolenic acids), the plants described above may have a reduced 18:1 fatty acid content of the seed oil fraction.

### 3.0 Characteristics of Plants Bearing Chromosome N12 and/or Chromosome N17 Variations Leading to Elevated Levels of 18:3 Fatty Acids in the Seed Oil Fraction and the Composition of their Seed Oil Fraction

Non-transgenic Brassicaceae having elevations in the 18:3 fatty acid content of their seed oil fraction can be developed through the use of mutagenesis as described above. In some embodiments, B. napus, B. oleracea, and/or B. juncea plants, lines or varieties having elevated levels of 18:3 fatty acid can be derived by cross breeding of the 18:3 content trait(s) induced by mutagenesis, such as those of rrrm1367-003 or its progeny, into other plant lines and varieties of those species.

In some embodiments, the 18:3 fatty acid content of plants having modifications on chromosome N12 and/or chromosome N17, and seed oil from those plants, may be described relative to reference plants grown under the same or substantially the same conditions and/or the seed oil from the reference plants.

In one embodiment, the seed oil of the plants has an elevated 18:3 (e.g., alpha and/or gamma linolenic acid) fatty acid content in the seed oil fraction that is greater than 1.4, 1.5, 1.6, 1.8, 2.0, or 2.2 times higher than a reference plant selected from B. napus cv. Topas, ATCC deposit PTA-120738, or B. napus cv. AV-Sapphire (breeders code RO011), where the plant and the reference plant (reference strain or line) are grown under the same or substantially the same conditions, and said seed is harvested under the same or substantially the same conditions. In another embodiment, the 18:3 (e.g., alpha and/or gamma linolenic acid) fatty acid content in the seed oil fraction is greater than 1.4, 1.5, 1.6, 1.7, 1.8, 1.9, 2.0, 2.1, 2.2, 2.3, 2.4, 2.5, 2.6, 2.8, or 3 times higher than the reference B. napus cv. Topas, wherein said reference is grown under the same or substantially the same conditions, and said seed is harvested under the same or substantially the same conditions. In another embodiment, the 18:3 (e.g., alpha and/or gamma linolenic acid) fatty acid content in the seed oil fraction is greater than 1.4, 1.5, 1.6, 1.8, 1.9, 2.0, 2.1, 2.2, 2.3, or 2.4 times higher than the reference B. napus cv. AV-Sapphire, wherein said reference is grown under the same or substantially the same conditions, and said seed is harvested under the same or substantially the same conditions. When evaluating the plants, seed, or seed oil described herein, relative to a reference plant (reference strain or line) its seed or seed oil, the plants are grown under the same or substantially the same conditions, and said seed is harvested under the same or substantially the same conditions (e.g., same number of days following planting). The reference line B. napus cv. AV-Sapphire (breeders code RO011) was released by Agriculture Victoria Services in association with GRDC and marketed by Dovuro Seeds under a license from Monsanto Australia Ltd. As previously indicated, the Topas line has been deposited with the ATCC and designated ATCC deposit PTA-120738.

In addition to being described relative to plants grown under the same or similar conditions, the 18:3 fatty acid content of plants having modifications on chromosome N12 and/or chromosome N17 that give rise to elevated 18:3 levels may be described in terms of the weight percent of the 18:3 fatty acids found in the oil fraction of those plants. Accordingly, plants, or parts thereof including seeds, having modifications on chromosome N12 and/or chromosome N17, such as those found in rrm1367-003, may have a 18:3 fatty acid content greater than or equal to 7, 8, 9, 10, 11, 12, 13, 14, 15, 16, 17, 18, 19, 20, 21, or 22 percent and an upper range limit of about 22 or 23 percent by weight, provided the upper range limit is greater than the lower range limit. Alternatively, the 18:3 (e.g., alpha and/or gamma linolenic acid) content may be in a range selected from 7-9, 9-12, 12-15, 15-19, 16-20, 17-21, 17-22, 18-22, or 19-23 percent by weight of the seed oil fraction. In another embodiment, the linoleic acid content of oil fraction may be less than 27, 26, 25, 24, 23, 22, 21, 20, 19, 18, 17, 16, or 15 percent by weight of the oil fraction or in a range selected from 26-24, 24-22, 22-20, 20-16, 19-15, 18-15, or 17-14 percent by weight of the seed oil fraction.

Plants having elevated 18:3 content due to the presence of alterations in the chromosome N12 and/or chromosome N17, such as those found in rrm1367-003, may also have alterations in the level of other fatty acids in the oil fraction. In one embodiment the seed oil fraction of such plants has an oleic acid content less than 69, 68, 66, 64, 62, 61, 60, 58, 56, 54, 52, 50, 48, 46, 44, or 42 percent by weight. In another embodiment, the seed oil fraction of such plants have an oleic acid content in a range selected from 69-60, 65-53, 60-50, or 50-41 percent by weight. In still another embodiment, the seed oil fraction has less than 2, 1.5, 1, 0.9, 0.8, 0.7, 0.6, 0.5, 0.4, 0.3, 0.2, or 0.1 percent erucic acid by weight.

In one embodiment, plants having elevated 18:3 content due to the presence of alterations on chromosome N12 and/or chromosome N17, such as those found in rrm1367-003, have an 18:3 fatty acid (alpha and/or gamma linolenic acid) content greater than 15 percent by weight of the fatty acids in the seed oil fraction, an oleic acid content in a range selected from 41-50 or 50-58 percent by weight, and a linoleic acid content in a range selected from 15-20, 18-22, or 20-24 percent by weight of the oil fraction.

In one embodiment, plants having elevated 18:3 content due to the presence of alterations on chromosome N12 and/or chromosome N17, such as those found in rrm1367-003, have an 18:3 fatty acid (alpha and/or gamma linolenic acid) content greater than 16 percent by weight of the fatty acids in the seed oil fraction, an oleic acid content in a range selected from 41-50, 45-55, or 50-60 percent by weight, and a linoleic acid content in a range selected from 15-20, 18-22, or 20-24 percent by weight.

In one embodiment, plants having elevated 18:3 content due to the presence of alterations on chromosome N12 and/or chromosome N17, such as those found in rrm1367-003, have, an 18:3 fatty acid (alpha and/or gamma linolenic acid) content greater than 17 percent by weight; an oleic acid content in a range selected from 44-50, 46-55, 45-56, 50-55, or 50-57 percent by weight, and a linoleic acid content in a range selected from 15-20, 18-22, or 20-24 percent by weight.

In one embodiment, this disclosure includes and provides for oil, an oil fraction, or a crush oil fraction, produced from plants, or parts thereof including seeds, having:
all or part of the genomic sequence of chromosome N12 between SNP markers C2-p16531874 and C2-p51360247 of B. napus line rrm1367-003 (e.g., the segments outlined in Table 1); and/or
all or part of the genomic sequence of chromosome N12 between SNP markers C7-p4690293 and C7-p22897297 of B. napus line rrm1367-003 (e.g., the segments outlined in Table 3);
wherein one or more of those genomic fragments can be detected in the oil.
   In one such embodiment, one or more of the genomic fragments present in the oil have been shown to result in elevated 18:3 fatty acid content in the seed oil fraction of a permissive cultivar such as “Topas” (e.g., when introduced by cross breeding). In such an embodiment, the all or part of the genomic sequence comprises one or more nucleic acid sequences having a length greater than 10, 25, 50, 100, 200, 300, 400, 500, 1,000, 1,500, 2,000, 3,000, 5,000, 7,500, 10,000, 20,000, 30,000, 50,000, 100,000, 500,000, or 1,000,000 nucleotides or a length in a range selected from 25-50, 25-100, 50-200, 100-500, 250-1,000, 500-5,000, 2,000-10,000, 5,000-20,000, 10,000-100,000, 50,000-400,000, 200,000-1,000,000 nucleotides.

In another embodiment, this disclosure includes and provides for plants and parts thereof, including seed, of B. napus rrm1367-003 deposited as ATCC Accession No. PTA-120636, and progeny thereof, having a seed oil fraction with a linolenic acid content greater than about 16, 17 18 19, 20, 21, or 22 percent.

### 4.0 Combining the Elevated 18:3 Fatty Acid Traits of Plants Bearing Chromosome N12 and/or Chromosome N17 Mutations with Additional Traits

Both non-transgenic and transgenic methods can be employed to combine the elevated 18:3 phenotype associated with mutations in the regions of chromosome N12 and/or chromosome N17 described herein (e.g., the mutations found in B. napus rrm1367-003), with one or more additional traits in plants of the Brassicaceae. Those additional traits can further influence the profile of fatty acids in the seed oil fraction or introduce other desirable phenotypic traits. Other traits that can be combined with the elevated 18:3 phenotype include, but are not limited to, increased resistance/tolerance to herbicides, insects, and various disease/pathogens (e.g., blackleg resistance conferred by the Rlm1, Rlm2, Rlm3, Rlm4, Rlm7, LepR2, and/or LepR3 gene), as well as drought resistance, and male sterility.

In one embodiment, the additional trait that is combined with the elevated 18:3 phenotype is the limited accumulation of erucic acid. Plants having less than 2, 1, 0.5, or 0.1 percent erucic acid by weight of the seed oil fraction can be obtained by cross breeding with plants known to have low erucic acid content.

In another embodiment, the additional trait that is combined with the elevated 18:3 phenotype is the limited accumulation of glucosinolates. Plants whose seed has a meal fraction that contains less than 8, 10, 15, 20, 25, 30, 35, or 40 micromoles of any one or more of 3-butenyl glucosinolate, 4-pentenyl glucosinolate, 2-hydroxy-3 butenyl glucosinolate, and 2-hydroxy-4-pentenyl glucosinolate per gram of dry (air-dry), oil-free solid can be obtained by cross breeding with plants known to have low erucic acid content.

In another embodiment, the additional trait that is combined with the elevated 18:3 phenotype is herbicide tolerance in plants or parts thereof, including cells, callus, or protoplast. That trait can be introduced by selection with the herbicide for which tolerance is sought, or by transgenic means where the genetic basis for the tolerance has been identified. Accordingly, tolerance to a herbicide selected from the group consisting of imidazolinone, dicamba, cyclohexanedione, sulfonylurea, glyphosate, glufosinate, phenoxy propionic acid, L-phosphinothricin, triazine and benzonitrile may be combined with the elevated 18:3 phenotype.

In another embodiment, the additional trait that is combined with the elevated 18:3 phenotype is insect resistance conferred by a gene encoding a Bacillus thuringiensis endotoxin that is expressed in said plant, part thereof, cell, or protoplast.

In still another embodiment, the additional trait that is combined with the elevated 18:3 phenotype is male sterility. Male sterility can be induced, for example, by cross breeding with male sterile lines.

### 5.0 Certain Embodiments

1. Seed of a Brassica napus, Brassica oleracea, or Brassica juncea plant comprising all or part of the genomic sequence of B napus line rrm1367-003 between SNP markers: C2-p1653187 and C2-p51360247;
wherein the part of the genomic sequence optionally is greater than 10, 25, 50, 100, 200, 300, 400, 500, 1,000, 1,500, 2,000, 3,000, 5,000, 7,500, 10,000, 20,000, 30,000, 50,000, 100,000, 500,000, or 1,000,000 base pairs or is in a range selected from 25-50, 25-100, 50-200, 100-500, 250-1,000, 500-5,000, 2,000-10,000, 5,000-20,000, 10,000-100,000, 50,000-400,000, 200,000-1,000,000 base pairs as described, for example in Section 2 of the present disclosure.
2. The seed of embodiment 1 comprising all or part of the genomic sequence between:
SNP markers C2-p1653187 and C2-p24304466 or C2-p24305313;
SNP markers C2-p24305313 and C2-p29505741 or C2-p29607300;
SNP markers C2-p29607300 and C2-p32147720 or C2-p32588191; and/or
SNP markers C2-p32588191 and C2-p51360247 of B. napus line rrm1367-003.
3. The seed of any preceding embodiment comprising all or part of the genomic sequence between:
SNP markers C2-p1653187 and C2-p21768270 or C2-p22394304;
SNP markers C2-p21768270 and C2-p24304466 or C2-p24305313;
SNP markers C2-p24305313 and C2-p28031338 or C2-p28070964;
SNP markers C2-p28031338 and C2-p29505741 or C2-p29607300;
SNP markers C2-p29607300 and C2-p30902832 or C2-p30942623;
SNP markers C2-p30902832 and C2-p32147720 or C2-p32588191
SNP markers C2-p32588191 and C2-p34723961 or C2-p34766378; and/or
SNP markers C2-p34723961 and C2-p51360247.
4. The seed of any preceding embodiment comprising all or part of the genomic sequence between any two SNP markers selected from the group consisting of: C2-p1653187, C2-p17090347, C2-p18795892, C2-p18859540, C2-p19649557, C2-p19840955, A02-p13167989, C2-p20927460, C2-p21691691, C2-p21735536, C2-p21768270, C2-p22394304, C2-p22396332, C2-p22448670, C2-p22466687, C2-p22481832, C2-p22587309, C2-p22588899, C2-p22638585, C2-p22736506, C2-p22807447, C2-p24304466, C2-p24305313, C2-p25019477, C2-p25478505, C2-p25656807, C2-p25913678, C2-p26147167, C2-p26159348, C2-p26207733, C2-p27157822, C2-p27601989, C2-p28031338, C2-p28070964, C2-p28698152, C2-p28806917, C2-p29076828, C2-p29348165, C2-p29383684, SC00434-p169753, C2-p29474845, C2-p29474845, C2-p29505033, C2-p29505741, C2-p29607300, C2-p29984659, C2-p30062266, C2-p30070472, C2-p30110169, C2-p30154901, C2-p30162991, C2-p30402845, C2-p30431524, C2-p30771286, C2-p30902832, C2-p30942623, C2-p31035160, C2-p31230778, C2-p31354336, C2-p31475220, C2-p31485080, C2-p31502391, C2-p31807771, C2-p31985379, C2-p32008623, C2-p32147720, C2-p32588191, C2-p3353696791, C2-p33633673, C2-p33653822, C2-p33745239, C2-p33761702, C2-p33897506, C2-p33982349, C2-p34550916, C13529254-p142, C2-p34723961, C2-p34766378, C2-p35082231, C2-p35629571, C2-p36261423, C2-p36532052, C2-p36905514, C2-p37181623, C2-p38415038, A02-p21713756 A02-p25181726, and C2-p51360247.
5. The seed of any preceding embodiment comprising all or part of the genomic sequence between any two SNP markers selected from the group consisting of:
C2-p25019477, C2-p25478505, C2-p25656807, C2-p25913678, C2-p26147167, C2-p26159348, C2-p26207733, C2-p27157822, C2-p27601989, C2-p28031338, C2-p28070964, C2-p28698152, C2-p28806917, C2-p29076828, C2-p29348165, C2-p29383684, SC00434-p169753, C2-p29474845, C2-p29474845, C2-p29505033, C2-p29505741, C2-p29607300, C2-p29984659, C2-p30062266, C2-p30070472, C2-p30110169, C2-p30154901, C2-p30162991, C2-p30402845, C2-p30431524, C2-p30771286, C2-p30902832, C2-p30942623, C2-p31035160, C2-p31230778, C2-p31354336, C2-p31475220, C2-p31485080, C2-p31502391, C2-p31807771, C2-p31985379, C2-p32008623, C2-p32147720, C2-p32588191, C2-p3353696791, C2-p33633673, C2-p33653822, C2-p33745239, C2-p33761702, C2-p33897506, C2-p33982349, C2-p34550916, C13529254-p142, C2-p34723961, C2-p34766378, C2-p35082231, C2-p35629571, C2-p36261423, C2-p36532052, C2-p36905514, C2-p37181623, C2-p38415038, A02-p21713756 A02-p25181726, and C2-p51360247.
6. The seed of embodiment 1, comprising all or part of the genomic sequence of chromosome N12 between any two SNP markers selected from the group consisting of C2-p22807447, C2-p24304466, C2-p24305313, C2-p25019477, C2-p25478505, C2-p25656807, C2-p25913678, C2-p26147167, C2-p26159348, C2-p26207733, C2-p27157822, C2-p27601989, C2-p28031338, C2-p28070964, C2-p28698152, C2-p28806917, C2-p29076828, C2-p29348165, C2-p29383684, SC00434-p169753, C2-p29474845, C2-p29474845, C2-p29505033, C2-p29505741, C2-p29607300, C2-p29984659, C2-p30062266, C2-p30070472, C2-p30110169, C2-p30154901, C2-p30162991, C2-p30402845, C2-p30431524, C2-p30771286, C2-p30902832, C2-p30942623, C2-p31035160, C2-p31230778, C2-p31354336, C2-p31475220, C2-p31485080, C2-p31502391, C2-p31807771, C2-p31985379, C2-p32008623, C2-p32147720, C2-p32588191, C2-p3353696791, C2-p33633673, C2-p33653822, C2-p33745239, C2-p33761702, C2-p33897506, C2-p33982349, C2-p34550916, C13529254-p142, C2-p34723961, C2-p34766378, C2-p35082231, C2-p35629571, C2-p36261423, C2-p36532052, C2-p36905514, C2-p37181623, C2-p38415038, A02-p21713756 A02-p25181726, and C2-p51360247.
7. A seed of a B. napus, B. oleracea, or B. juncea plant comprising all or part of the genomic sequence of B. napus line rrm1367-003 between SNP markers: C7-p4690293 and C7-p22897297;
wherein the part of the genomic sequence optionally is greater than 10, 25, 50, 100, 200, 300, 400, 500, 1,000, 1,500, 2,000, 3,000, 5,000, 7,500, 10,000, 20,000, 30,000, 50,000, 100,000, 500,000, or 1,000,000 base pairs or is in a range selected from 25-50, 25-100, 50-200, 100-500, 250-1,000, 500-5,000, 2,000-10,000, 5,000-20,000, 10,000-100,000, 50,000-400,000, 200,000-1,000,000 base pairs as described, for example in Section 2 of the present disclosure.
8. The seed of embodiment 7 comprising all or part of the genomic sequence between:
SNP markers C7-p4690293 and C7-p9593996 or C7-p10040604;
SNP markers C7-p10040604 and C7-p12072579 or C7-p12079142;
SNP markers C7-p12079142 and C7-p12512146 or C7-p12514520; and/or
SNP markers C7-p12514520 and C7-p22897297 of B. napus line rrm1367-003.
9. The seed of any of embodiments 7 to 8 comprising all or part of the genomic sequence between:
SNP markers C7-p4690293 and C7-p8719053, C7-p8726636;
SNP markers C7-p8726636 and C7-p9593996 or C7-p10040604;
SNP markers C7-p10040604 and C7-p10215325 or C7-p10228536;
SNP markers C7-p10228536 and C7-p12072579 or C7-p12079142;
SNP markers C7-p12079142 and C7-p12300699, C7-p12301957;
SNP markers C7-p12301957 and C7-p12512146 or C7-p12514520;
SNP markers C7-p12514520 and C7-p12995305 or C7-p13029440; and/or
SNP markers C7-p12995305 and C7-p22897297.
10. The seed of any of embodiments 7 to 9 comprising all or part of the genomic sequence between any two SNP markers selected from the group consisting of: C7-p4690293, C7-p5039845, C7-p5194981, C7-p7498659, C7-p8599974, C7-p8719053, C7-p8726636, C7-p8726743, C7-p8727745, C7-p8766230, C7-p8824122, C7-p8854349, C7-p8870860, C7-p9307503, C7-p9358459, C7-p9593996, C7-p10040604, C7-p10165832, C7-p10180076, C7-p10180716, C7-p10212158, C7-p10215060, C7-p10215325, C7-p10228536, C7-p10261396, C7-p10262047, C7-p10613314, C7-p10617039, C7-p10720977, C7-p11706153, C7-p11718201, C7-p12072579, C7-p12079142, C7-p12123100, C7-p12123399, C7-p12268682, C7-p12281546, C7-p12300699, C7-p12300699, C7-p12301957, C7-p12356302, C7-p12356455, C7-p12385657, C7-p12387173, C7-p12401233, C7-p12485308, C7-p12508706, C7-p12512146, C7-p12514520, C7-p12565005, C7-p12684624, C7-p12757060, C7-p12984513, C7-p12990275, C7-p12995305, C7-p13029440, C7-p13029555, C7-p13069990, C7-p13070860, C7-p13083371, C7-p13135120, C7-p22861548, C7-p22870500 and C7-p22897297.
11. The seed of any of embodiments 7 to 10 comprising all or part of the genomic sequence between any two SNP markers selected from one or more of the groups consisting of: C7-p4690293, C7-p5039845, C7-p5194981, C7-p7498659, C7-p8599974, C7-p8719053, C7-p8726636, C7-p8726743, C7-p8727745, C7-p8766230, C7-p8824122, C7-p8854349, C7-p8870860, C7-p9307503, C7-p9358459, C7-p9593996, C7-p10040604, C7-p10165832, C7-p10180076, C7-p10180716, C7-p10212158, C7-p10215060, C7-p10215325, C7-p10228536, C7-p10261396, C7-p10262047, C7-p10613314, C7-p10617039, C7-p10720977, and C7-p11706153;
C7-p11706153, C7-p11718201, C7-p12072579, C7-p12079142, C7-p12123100, C7-p12123399, C7-p12268682, C7-p12281546, C7-p12300699, C7-p12300699, C7-p12301957, C7-p12356302, C7-p12356455, C7-p12385657, C7-p12387173, C7-p12401233, C7-p12485308, C7-p12508706, C7-p12512146, C7-p12514520, C7-p12565005, C7-p12684624, C7-p12757060, C7-p12984513, C7-p12990275, C7-p12995305, C7-p13029440, C7-p13029555, C7-p13069990, C7-p13070860, C7-p13083371, and C7-p13135120; or
C7-p13135120, C7-p22861548, C7-p22870500, and C7-p22897297.
12. The seed of any preceding embodiment comprising all or part of the genomic sequence of B. napus rrm1367-003 between any two SNP markers set forth in embodiments 1 to 6, and/or all or part of the genomic sequence of B. napus rrm1367-003 between any two SNP markers set forth in embodiments 7 to 11.
13. The seed of any preceding embodiment comprising all or part of the genomic sequence of B. napus rrm1367-003 between SNP markers: C2-p23082339 and C2-p23898427, C2-p32635329 and C2-p32643944, C2-p37254117 and C2-p37285344, C2-p41012763 and C2-p51360247.
14. The seed of any preceding embodiment comprising all or part of the genomic sequence of B. napus rrm1367-003 between SNP markers: C2-p1653187 and C2-p51360247; and/or C7-p4690293 and C7-p2287297, which genomic sequence when introduced into B. napus cv. Topas, ATCC deposit PTA-120738, results in an increase in the 18:3 content of the seed oil fraction of seeds produced by the plant into which the fragment has been introduced (e.g., by breeding) relative to B. napus cv. Topas grown under the same or substantially the same conditions.
15. The seed of any preceding embodiment wherein at least one part of the genomic sequence of B. napus rrm1367-003 present in said seed has a length greater than 10, 25, 50, 100, 200, 300, 400, 500, 1,000, 1,500, 2,000, 3,000, 5,000, 7,500, 10,000, 20,000, 30,000, 50,000, or 100,000 base pairs or is in a range selected from 25-50, 25-100, 50-200, 100-500, 250-1,000, 500-5,000, 2,000-10,000 and 5,000-10,000 base pairs.
16. The seed of any preceding embodiment, wherein the alpha linolenic acid content is greater than 1.4 times higher than a reference strain selected from: B. napus cv. Topas; or B. napus cv. AV-Sapphire, breeders code RO011; wherein said reference strain is grown under the same or substantially the same conditions, and said seed is harvested under the same or substantially the same conditions.
17. The seed of any preceding embodiment wherein the alpha linolenic acid content is greater than 1.4, 1.5, 1.6, 1.7, 1.8, 1.9, 2.0, 2.1, 2.2, or 2.3 times higher than the reference strain B. napus cv. Topas, wherein said reference strain is grown under the same or substantially the same conditions, and said seed is harvested under the same or substantially the same conditions.
18. The seed of any preceding embodiment wherein the alpha linolenic acid content is greater than 1.4, 1.5, or 1.6 times higher than the reference strain B. napus cv. AV-Sapphire, breeders code RO011, wherein said reference strain is grown under the same or substantially the same conditions, and said seed is harvested under the same or substantially the same conditions.
19. The seed of any preceding embodiment wherein the seed has an oil fraction with an 18:3 fatty acid content greater than 7, 8, 9, 10, 11, 12, 13, 14, 15, 16, 17, 18, 19, 20, 21, or 22 percent by weight of the oil fraction, or in a range selected from 7-9, 9-12, 12-15, 15-19, or 19-23 percent by weight of the oil fraction.
20. The seed of embodiment 19, wherein the alpha linolenic acid content is in a range selected from greater than 7, 8, 9, 10, 11, 12, 13, 14, 15, 16, 17, 18, 19, 20, 21, or 22 percent by weight of the oil fraction, or in a range selected from 7-9, 9-12, 12-15, 15-19, or 19-23 percent by weight of the oil fraction.
21. The seed of any preceding embodiment wherein the seed has an oil fraction with a linolenic acid content in a range selected from 26-24, 24-22, 22-20, 20-16, 19-15, 18-15, or 17-14 percent by weight.
22. The seed of any preceding embodiment wherein the seed has an oil fraction with an oleic acid content less than 69, 68, 66, 64, 62, 61, 60, 58, 56, 54, 52, 50, 48, 46, 44, or 42 percent by weight or in a range selected from 69-60, 65-53, 60-50, or 50-41 percent by weight.
23. The seed of any preceding embodiment, wherein the seed has an oil fraction with:
a linolenic (e.g., alpha linolenic and/or gamma linolenic) acid content greater than 15 percent by weight; and
an oleic acid content in a range selected from 41-50 or 50-58 percent by weight.
   24. The seed of embodiment 23, wherein the seed has an oil fraction with a linolenic acid content in a range selected from 15-20, 16-21, 17-22, 18-23 or 20-24 percent by weight.
   25. The seed of any preceding embodiment, wherein the seed has an oil fraction with:
a linolenic (e.g., alpha linolenic and/or gamma linolenic) acid content greater than 16 percent by weight;
an 18:1 fatty acid content in a range selected from 41-50, 45-55, or 50-60 percent by weight; and
an 18:2 fatty acid content in a range selected from 15-20 or 20-24 percent by weight.
   26. The seed of any preceding embodiment, wherein the seed has an oil fraction with:
a linolenic (e.g., alpha linolenic) acid content greater than 17 percent by weight;
an 18:1 fatty acid content in a range selected from 44-50, 46-55, 45-56, 50-55, or 50-57 percent by weight; and
an 18:2 fatty acid in a range selected from 15-20 or 20-24 percent by weight.
27. The seed of any preceding embodiment, having less than 4, 3, 2, or 1 percent erucic acid by weight of the seed oil fraction.
28. A plant grown from the seed of any preceding embodiment or a part thereof, wherein said plant is non-transgenic, transgenic, or transgenic subject to the proviso that the only transgenes present are genes for herbicide resistance.
29. A part of the plant of embodiment 28, selected from the group consisting of leaf, pollen, ovule, embryo, cotyledon, hypocotyl, meristematic cell, root, root tip, pistil, anther, flower, seed, shoot, stem, pod and petiole.
30. A cell, protoplast or tissue of a plant of embodiments 28-29.
31. A plant or part thereof, including a cell, protoplast or tissue, according to any of embodiments 28-30 expressing a herbicide tolerance.
32. The plant, part thereof, cell, protoplast or tissue of embodiment 31, wherein said the herbicide tolerance is tolerance to a herbicide selected from the group consisting of imidazolinone, dicamba, cyclohexanedione, sulfonylurea, glyphosate, glufosinate, phenoxy propionic acid, L-phosphinothricin, triazine and benzonitrile.
33. A plant, part thereof, cell, protoplast or tissue of any of embodiments 28-32, having insect resistance conferred by a gene encoding a Bacillus thuringiensis endotoxin which is expressed in said plant, part thereof, cell, protoplast or tissue.
34. The plant of any of embodiments 28-33, wherein the seed has a meal fraction that contains less than 10, 15, 20, 25, 30, 35, or 40 micromoles of any one or more of 3-butenyl glucosinolate, 4-pentenyl glucosinolate, 2-hydroxy-3 butenyl glucosinolate, and 2-hydroxy-4-pentenyl glucosinolate per gram of dry (air-dry), oil-free solid.
35. The plant of any of embodiments 28-33, wherein said plant is male sterile.
36. Oil from a seed of embodiments 1-27, or a plant, or part thereof, grown from a seed of embodiments 1-27, wherein said oil comprises nucleic acids having all or part of the genomic sequence of B. napus line rrm1367-003.
37. The oil of embodiment 36, wherein the oil is produced from a seed of embodiment 14 and comprises a genomic sequence that when introduced B. napus cv. Topas (e.g., by cross breeding), ATCC deposit PTA-120738, results in an increase in the 18:3 content of the seed oil fraction of seeds produced by the plant into which the fragment has been introduced (e.g., by breeding) relative to B. napus cv. Topas grown under the same or substantially the same conditions.
38. Brassica napus rrm1367-003 deposited as ATCC Accession number PTA-120636.
39. An isolated nucleic acid which comprises all or part of the genomic sequence: between SNP markers C2-p1653187 and C2-p51360247 of B. napus line rrm1367-003 as set forth in embodiments 4 and 5; and/or between SNP markers C7-p4690293 and C7-p22897297 of B. napus line rrm1367-003 as set forth in embodiments 10 or 11.
40. The isolated nucleic acid of embodiment 39, wherein said isolated nucleic acid when introduced B. napus cv. Topas, ATCC deposit PTA-120738, results in an increase in the 18:3 fatty acid content (e.g., alpha and/or gamma linolenic acid) of the seed oil fraction of seeds produced by the plant into which the fragment has been introduced (e.g., by breeding) relative to B. napus cv. Topas grown under the same or substantially the same conditions.
41. The nucleic acid of embodiment 39 or 40, wherein said isolated nucleic acid is free of viable Brassica cells.
42. The nucleic acid of any of embodiments 39-41, wherein said nucleic acid is in the form of a plasmid or vector that comprises an origin of replication for the propagation of the nucleic acid.
43. The nucleic acid of embodiment 42, wherein the vector is a cosmid, yeast artificial chromosome(s) (YAC(s)), a phage, or a virus.
44. A non-transgenic B. napus, B. olereca, or B. juncea plant, or parts thereof, having a low-saturated-fat trait that produces seed having an oil fraction with a linolenic acid content of at least 15, 16, 17, 18, 19, 20, 21, or 22 percent by weight.
45. The plant of embodiment 44, wherein the plant is a B. napus plant.
46, The plant of embodiment 44 or 45, wherein the oil fraction has an erucic acid content of less than 2, 1.8, 1.6, 1.4, 1.2, 1.0, or 0.8 percent by weight of the seed oil fraction.
47. The plant of embodiment 46, wherein the oil fraction has an erucic acid content of less than 1.2, 1.0, or 0.8 percent by weight of the seed oil fraction.
48. The plant of any of embodiments 44-47, wherein the seed has a meal fraction that contains less than 10, 15, or 20 micromoles of any one or more of 3-butenyl glucosinolate, 4-pentenyl glucosinolate, 2-hydroxy-3 butenyl glucosinolate, and 2-hydroxy-4-pentenyl glucosinolate per gram of dry (air-dry), oil-free solid.
49. The plant of any of embodiments 44-48, wherein the plant has a transgenic herbicide tolerance, preferably to a herbicide selected from the group consisting glyphosate, glufosinate, or imidazolinone.
50. A seed or plant cell from a B. napus, B. olereca, or B. juncea plant whose seed has an oil fraction and a meal fraction, the oil fraction having a linolenic acid content of at least 15, 16, 17, 18, 19, 20, 21, or 22 percent by weight and the meal fraction being free of any transgenes other than transgenes conferring herbicide tolerance.
51. The seed or plant cell of embodiment 50, wherein said meal fraction further comprises a transgene conferring insect resistance.
52. A plant produced by the seed of embodiment 50 or 51.
53. The progeny of a plant of embodiment 52, wherein the seed has an oil fraction with a linolenic acid content of at least 15, 16, 17, 18, 19, 20, 21, or 22 percent by weight.
54. A method for making a Brassica plant, said method comprising:

a) obtaining one or more first Brassica parent plants comprising all or part of the genomic sequence of B. napus line rrm1367-003 between SNP markers: C2-p1653187 and C2-p51360247, and/or all or part of the genomic sequence of B. napus line rrm1367-003 between SNP markers: C7-p4690293 and C7-p22897297;

b) obtaining one or more second Brassica parent plants;

c) crossing said one or more first Brassica parent plants and said one or more second Brassica parent plants; and

d) selecting, for one to five generations, for progeny plants having an increased level of linolenic acid.
55. The method of embodiment 54, wherein said linolenic acid is alpha linolenic acid.
56. The method of embodiment 54 or 55, wherein each said part of the genomic sequence of B. napus line rrm1367-003 has a length that is independently selected from a length that is greater than 10, 25, 50, 100, 200, 300, 400, 500, 1,000, 1,500, 2,000, 3,000, 5,000, 7,500, 10,000, 20,000, 30,000, 50,000, 100,000, 500,000, or 1,000,000 base pairs or is in a range selected independently from 25-50, 25-100, 50-200, 100-500, 250-1,000, 500-5,000, 2,000-10,000, 5,000-20,000, 10,000-100,000, 50,000-400,000, 200,000-1,000,000 base pairs.

### 6.0 EXAMPLES

### Example 1

### Determination of the Fatty Acid Composition of Oil from Seeds

The fatty acid composition of seeds is determined by a modification of American Oil Chemist's Society (AOCS) protocol Ce le-91. In the procedure fatty acids present as acylglycerols are converted to fatty acid methyl esters, which are analyzed by gas liquid chromatography (GLC or GC). For each sample to be analyzed 20-30 seeds are placed in a 15 ml centrifuge to along with two steel ball bearings. The tube is capped and shaken for one minute or until the seeds are visibly crushed. Approximately 0.6 mL of 1 N KOH in methanol is added to the tube, and the tube is shaken again for approximately 30 seconds. The tube and its contents are placed in a water bath at 60±5° C. for 1 min. After removing the tube from the bath 4 mL of saturated sodium chloride and 2.5 mL of isooctane are added, the tube is shaken and centrifuged for 1 min. in a tabletop centrifuge. A portion of the isooctane supernatant is transferred to a gas chromatographic (GC) vial and capped. Vials are stored at 0-4° C. until analysis, but no more than five days.

Fatty acid methyl esters were 1 subject to analysis on a GC on an instrument equipped with a DB 23 column from VWR International modified with 50% cyanopropyl and 50% methylpolysiloxane (or an equivalent stationary phase suitable for the separation) 5 meters long×with a 180 micron diameter and 20 micron bore and a flame ionization detector. The instrument is calibrated with a fatty acid methyl ester standard, such as NuChek Prep Catalog number GLC 432.

The content of fatty acids having from 14 carbon atoms (C14 fatty acids) to 24 carbon atoms (C24 fatty acids) is determined using the integrated peak area for each type of fatty acid reported normalized to the total peak area for those fatty acids as 100% to determine their percent by weight.

### Example 2

### Development of B. napus Lines with Elevated 18:3 Content by UV Irradiation of Microspores

Microspores of B. napus cv. Topas were isolated and suspended in NLN-13 medium with 0.05% colchicine in culture dishes. The culture dishes were placed on the screen of the DNA Transilluminator (FOTODYE, Mode no. 3-3000, 300 nm, 15 W×4) in the dark and set for 20-30 minutes so that the microspores could settle to the bottom of the Petri dish. The UV light was turned on for 1.5-2 minutes, after which the culture dishes were wrapped with aluminum foil and immediately place them in 33° C. incubator for routine culture and the generation of doubled haploid plants.

Approximately 90% of the microspores were not viable after the UV irradiation, and from the remaining viable microspores 850 DH0 plants/lines were generated. DHa plants were generated from each DH0 line, and seed from the DH1 plants were grown to prepare DH2 seed from 847 DH2 plants for chemical analysis. The fatty acid profile of DH2 plant seeds indicates the presence of plants with elevated 18:3 content within the population as the maximum 18:3 level attained was 20.54% (Table 4).

TABLE 4
Summary of Fatty Acid Analysis of DH2 Seeds from UV Mutagenesis

| | 16:0 | C16:1 | C18:0 | C18:1 | C18:2 | C18:3 | C20:0 | C20:1 | SUM |
|---|---|---|---|---|---|---|---|---|---|
| Maximum | 6.58 | 0.63 | 4.15 | 68.13 | 24.72 | 20.54 | 1.39 | 1.77 | 100.00 |
| Minimum | 3.46 | 0.00 | 1.24 | 49.87 | 13.91 | 6.66 | 0.34 | 0.62 | 97.19 |
| Average | 4.17 | 0.21 | 1.76 | 62.12 | 19.04 | 9.35 | 0.68 | 1.39 | 99.59 |

### Example 3

### Development of Brassica juncea Lines with Elevated 18:3 Content by Radiation Mutagenesis

Radiation mutagenesis was conducted on a low erucic acid producing Russian B. juncea line designated DZJ01. After radiation treatment the seeds, designated M₁, were grown in greenhouse, allowed to openly pollinate other plants grown from the M₁ seed. M₂ seeds were harvested from the plants grown from M₁ seeds, and the M₂ seeds were sown in an open field. Approximately 1,000 plants were bagged to obtain M₃ seeds by self-pollination. Analysis of seed from the M₃ mutant lines resulted in several plants having significantly elevated 18:3 fatty acid content in their seed at levels exceeding the 95% confidence interval (17.43%) of the C16-C22 fatty acids (Table 5).

TABLE 5

| | C16:0 | C18:0 | C18:1 | C18:2 | C18:3 | C20:1 | C22:1 | SATS |
|---|---|---|---|---|---|---|---|---|
| SAMPLE | | | | | | | | |
| FTG614224 | 4.6 | 1.57 | 43.03 | 32.08 | 14.81 | 1.12 | 0.06 | 7.23 |
| FTG614285 | 3.81 | 1.67 | 41.5 | 34.96 | 14.93 | 1.23 | 0.04 | 6.46 |
| FTG614310 | 4.19 | 1.98 | 48.61 | 33.13 | 9.24 | 1.13 | 0.02 | 7.13 |
| FTG614311 | 3.86 | 1.53 | 39.52 | 35.87 | 16.29 | 1.23 | 0.04 | 6.29 |
| FTG614397 | 3.77 | 2.33 | 48.43 | 28.4 | 13.15 | 1.3 | 0.04 | 7.36 |
| FTG614425 | 5.32 | 0.04 | 44.76 | 34.77 | 10.71 | 1.06 | 0.11 | 6.43 |
| FTG614459 | 3.53 | 2.06 | 48.79 | 30.52 | 12.16 | 1.15 | 0.07 | 6.58 |
| FTG614489 | 3.39 | 2.1 | 48.38 | 30.28 | 12.72 | 1.29 | 0.05 | 6.55 |
| FTG614497 | 6.25 | 2.1 | 26.08 | 41.33 | 19.8 | 1.05 | 0.08 | 10 |
| FTG614499 | 3.58 | 2.2 | 50.37 | 28.99 | 12.06 | 1.19 | 0.03 | 6.78 |
| FTG614522 | 3.83 | 2.22 | 48.63 | 30.49 | 11.77 | 1.2 | 0 | 7.16 |
| FTG614689 | 3.54 | 1.94 | 45.65 | 30.59 | 15.19 | 1.32 | 0.03 | 6.49 |
| FTG614727 | 4.95 | 1.59 | 42.7 | 37.12 | 10.68 | 1.05 | 0.05 | 7.47 |
| FTG614731 | 3.98 | 1.6 | 38.76 | 34.25 | 18.12 | 1.26 | 0.05 | 6.6 |
| FTG614751 | 4.34 | 1.6 | 35.63 | 33.14 | 21.05 | 1.33 | 0.07 | 7.16 |
| FTG614752 | 6.56 | 2.05 | 26.92 | 37.2 | 22.49 | 1.03 | 0.07 | 10.44 |
| FTG614767 | 4.85 | 1.59 | 32.73 | 40.84 | 16.87 | 1.03 | 0.05 | 7.33 |
| FTG614793 | 5.34 | 0.03 | 43.24 | 34.2 | 12.52 | 1.1 | 0.13 | 6.6 |
| FTG614816 | 3.31 | 2.09 | 50.78 | 28.94 | 11.41 | 1.31 | 0.04 | 6.45 |
| FTG614857 | 5.27 | 1.97 | 34.63 | 35.2 | 19.12 | 1.2 | 0.04 | 8.5 |
| FTG615004 | 3.73 | 1.76 | 44.71 | 33.33 | 13.69 | 1.17 | 0.05 | 6.36 |
| Total = 840 | | | | | | | | |
| Average | 4.85 | 2.15 | 39.19 | 36.11 | 13.82 | 1.17 | 0.05 | 8.29 |
| Standard | 0.73 | 0.33 | 4.11 | 3.04 | 1.84 | 0.11 | 0.06 | 1.12 |
| Deviation | | | | | | | | |
| 95%− | 3.41 | 1.51 | 31.14 | 30.15 | 10.22 | 0.96 | −0.07 | 6.11 |
| 95%+ | 6.29 | 2.78 | 47.24 | 42.06 | 17.43 | 1.39 | 0.17 | 10.48 |

Selected lines from the M₃ generation were planted in greenhouse to yield M₄ progeny. Analysis of the seed fatty acid content of the M₄ progeny demonstrated that seed from several of the lines contained greater than 15% 18:3 fatty acids. A number of plants were in the 16-22.6%, 17-22.7%, or 18-22.6% range (all measures are based on the average fatty acid content). See Table 6.

TABLE 6

| SAMPLE ID | RESCHID | C16:0 | C18:0 | C18:1 | C18:2 | C18:3 | C22:1 | SATS |
|---|---|---|---|---|---|---|---|---|
| FTG650354 | M3_DZJ-310 | 4.21 | 2.05 | 50.75 | 30.63 | 9.27 | 0.04 | 7.30 |
| FTG650460 | M3_DZJ-310 | 4.20 | 2.05 | 49.84 | 31.38 | 9.61 | 0.05 | 7.27 |
| FTG650467 | M3_DZJ-310 | 4.69 | 2.04 | 47.05 | 33.28 | 9.78 | 0.07 | 7.84 |
| FTG650476 | M3_DZJ-310 | 4.17 | 2.13 | 50.19 | 31.18 | 9.27 | 0.05 | 7.36 |
| FTG650483 | M3_DZJ-310 | 4.40 | 1.90 | 49.30 | 31.71 | 9.59 | 0.06 | 7.30 |
| FTG650484 | M3_DZJ-310 | 4.12 | 2.12 | 49.28 | 31.90 | 9.37 | 0.06 | 7.32 |
| FTG650531 | M3_DZJ-310 | 4.26 | 2.07 | 49.70 | 31.59 | 9.30 | 0.04 | 7.40 |
| FTG650534 | M3_DZJ-310 | 4.24 | 2.27 | 50.99 | 30.74 | 8.86 | 0.04 | 7.56 |
| FTG650538 | M3_DZJ-310 | 4.10 | 1.99 | 50.14 | 31.14 | 9.61 | 0.06 | 7.10 |
| FTG650543 | M3_DZJ-310 | 4.40 | 2.34 | 50.32 | 31.35 | 8.60 | 0.06 | 7.82 |
| FTG650353 | M3_DZJ-311 | 4.11 | 2.34 | 48.54 | 28.18 | 13.51 | 0.05 | 7.57 |
| FTG650355 | M3_DZJ-311 | 4.13 | 2.24 | 48.27 | 28.29 | 13.80 | 0.06 | 7.51 |
| FTG650466 | M3_DZJ-311 | 4.39 | 2.57 | 44.85 | 30.98 | 13.71 | 0.08 | 8.31 |
| FTG650468 | M3_DZJ-311 | 4.32 | 2.02 | 47.46 | 29.06 | 13.93 | 0.07 | 7.47 |
| FTG650471 | M3_DZJ-311 | 4.11 | 2.50 | 48.87 | 28.07 | 13.12 | 0.08 | 7.84 |
| FTG650475 | M3_DZJ-311 | 4.24 | 2.48 | 46.50 | 29.33 | 14.14 | 0.07 | 7.99 |
| FTG650477 | M3_DZJ-311 | 4.24 | 2.36 | 47.10 | 29.28 | 13.69 | 0.07 | 7.82 |
| FTG650479 | M3_DZJ-311 | 4.15 | 2.20 | 46.73 | 29.41 | 14.27 | 0.06 | 7.48 |
| FTG650482 | M3_DZJ-311 | 4.25 | 2.32 | 48.45 | 28.13 | 13.58 | 0.06 | 7.76 |
| FTG650488 | M3_DZJ-311 | 4.17 | 2.42 | 46.23 | 29.90 | 13.90 | 0.06 | 7.84 |
| FTG650351 | M3_DZJ-425 | 4.41 | 2.57 | 47.40 | 31.77 | 10.67 | 0.05 | 8.20 |
| FTG650461 | M3_DZJ-425 | 4.70 | 2.84 | 49.33 | 31.55 | 8.40 | 0.04 | 8.79 |
| FTG650464 | M3_DZJ-425 | 4.63 | 2.69 | 50.69 | 30.30 | 8.63 | 0.05 | 8.53 |
| FTG650478 | M3_DZJ-425 | 4.56 | 2.45 | 50.31 | 30.67 | 9.09 | 0.05 | 8.08 |
| FTG650487 | M3_DZJ-425 | 4.55 | 2.39 | 50.32 | 30.72 | 9.07 | 0.05 | 8.01 |
| FTG650363 | M3_DZJ-459 | 3.82 | 2.86 | 49.90 | 28.18 | 11.96 | 0.05 | 7.91 |
| FTG650367 | M3_DZJ-459 | 4.04 | 2.36 | 48.47 | 30.77 | 11.18 | 0.04 | 7.55 |
| FTG650371 | M3_DZJ-459 | 4.15 | 2.57 | 45.76 | 31.38 | 12.94 | 0.04 | 7.90 |
| FTG650372 | M3_DZJ-459 | 3.82 | 2.22 | 51.29 | 26.96 | 12.67 | 0.04 | 7.10 |
| FTG650463 | M3_DZJ-459 | 4.15 | 2.40 | 48.44 | 30.12 | 11.52 | 0.05 | 7.85 |
| FTG650465 | M3_DZJ-459 | 3.76 | 2.52 | 50.14 | 28.14 | 12.31 | 0.06 | 7.43 |
| FTG650473 | M3_DZJ-459 | 3.85 | 2.65 | 50.78 | 27.21 | 12.14 | 0.07 | 7.71 |
| FTG650474 | M3_DZJ-459 | 4.26 | 2.62 | 46.06 | 30.39 | 13.12 | 0.07 | 8.23 |
| FTG650485 | M3_DZJ-459 | 3.90 | 2.92 | 50.59 | 27.49 | 11.81 | 0.05 | 8.15 |
| FTG650486 | M3_DZJ-459 | 3.81 | 2.67 | 50.99 | 27.24 | 11.96 | 0.05 | 7.77 |
| FTG650356 | M3_DZJ-489 | 3.66 | 2.60 | 51.18 | 27.03 | 12.13 | 0.05 | 7.50 |
| FTG650357 | M3_DZJ-489 | 3.70 | 2.54 | 50.39 | 27.43 | 12.56 | 0.04 | 7.44 |
| FTG650359 | M3_DZJ-489 | 3.67 | 2.30 | 50.43 | 27.73 | 12.48 | 0.05 | 7.14 |
| FTG650360 | M3_DZJ-489 | 3.65 | 2.82 | 52.77 | 26.07 | 11.19 | 0.06 | 7.77 |
| FTG650364 | M3_DZJ-489 | 3.65 | 2.64 | 50.53 | 27.39 | 12.35 | 0.05 | 7.51 |
| FTG650368 | M3_DZJ-489 | 3.66 | 2.51 | 49.63 | 28.45 | 12.28 | 0.05 | 7.39 |
| FTG650374 | M3_DZJ-489 | 3.57 | 2.59 | 51.36 | 26.99 | 12.10 | 0.05 | 7.35 |
| FTG650380 | M3_DZJ-489 | 3.62 | 2.57 | 51.11 | 26.95 | 12.47 | 0.04 | 7.35 |
| FTG650385 | M3_DZJ-489 | 3.71 | 2.56 | 52.09 | 26.73 | 11.73 | 0.04 | 7.40 |
| FTG650392 | M3_DZJ-489 | 3.66 | 2.73 | 51.58 | 26.86 | 11.83 | 0.05 | 7.59 |
| FTG650369 | M3_DZJ-499 | 3.97 | 2.63 | 49.24 | 28.94 | 11.94 | 0.04 | 7.80 |
| FTG650370 | M3_DZJ-499 | 3.90 | 2.34 | 49.64 | 28.36 | 12.45 | 0.06 | 7.40 |
| FTG650382 | M3_DZJ-499 | 4.03 | 2.56 | 49.45 | 28.72 | 11.99 | 0.05 | 7.77 |
| FTG650383 | M3_DZJ-499 | 3.91 | 2.38 | 49.71 | 28.11 | 12.71 | 0.04 | 7.39 |
| FTG650393 | M3_DZJ-499 | 4.07 | 2.24 | 48.99 | 29.18 | 12.35 | 0.05 | 7.39 |
| FTG650398 | M3_DZJ-499 | 3.97 | 2.44 | 48.91 | 29.39 | 12.04 | 0.05 | 7.57 |
| FTG650414 | M3_DZJ-499 | 3.90 | 2.41 | 49.91 | 28.30 | 12.23 | 0.04 | 7.48 |
| FTG650416 | M3_DZJ-499 | 3.94 | 2.43 | 49.52 | 28.47 | 12.31 | 0.06 | 7.55 |
| FTG650420 | M3_DZJ-499 | 3.89 | 2.29 | 49.60 | 28.42 | 12.52 | 0.04 | 7.37 |
| FTG650433 | M3_DZJ-499 | 3.85 | 2.28 | 47.84 | 29.95 | 12.70 | 0.06 | 7.30 |
| FTG650358 | M3_DZJ-689 | 3.89 | 2.39 | 50.38 | 26.95 | 13.08 | 0.06 | 7.46 |
| FTG650366 | M3_DZJ-689 | 3.84 | 2.58 | 50.40 | 26.85 | 13.00 | 0.06 | 7.59 |
| FTG650373 | M3_DZJ-689 | 4.03 | 2.18 | 49.37 | 27.13 | 14.01 | 0.04 | 7.31 |
| FTG650378 | M3_DZJ-689 | 3.89 | 2.29 | 49.92 | 27.49 | 13.11 | 0.05 | 7.30 |
| FTG650386 | M3_DZJ-689 | 4.03 | 2.54 | 50.59 | 25.87 | 13.76 | 0.04 | 7.74 |
| FTG650387 | M3_DZJ-689 | 3.97 | 2.30 | 48.29 | 28.09 | 14.03 | 0.05 | 7.41 |
| FTG650406 | M3_DZJ-689 | 3.93 | 2.14 | 49.79 | 27.10 | 13.80 | 0.05 | 7.15 |
| FTG650417 | M3_DZJ-689 | 3.85 | 2.47 | 49.44 | 26.96 | 14.03 | 0.05 | 7.50 |
| FTG650426 | M3_DZJ-689 | 4.08 | 2.37 | 49.01 | 28.12 | 13.12 | 0.07 | 7.63 |
| FTG650427 | M3_DZJ-689 | 3.91 | 2.54 | 50.55 | 26.66 | 13.07 | 0.05 | 7.64 |
| FTG650361 | M3_DZJ-731 | 3.69 | 2.66 | 50.89 | 26.68 | 12.77 | 0.05 | 7.55 |
| FTG650362 | M3_DZJ-731 | 3.95 | 2.08 | 46.78 | 28.88 | 15.07 | 0.06 | 7.09 |
| FTG650376 | M3_DZJ-731 | 3.84 | 2.25 | 51.23 | 28.15 | 11.35 | 0.04 | 7.19 |
| FTG650379 | M3_DZJ-731 | 3.96 | 2.18 | 49.04 | 27.93 | 13.71 | 0.04 | 7.22 |
| FTG650396 | M3_DZJ-731 | 3.82 | 2.69 | 50.58 | 27.61 | 12.15 | 0.04 | 7.65 |
| FTG650408 | M3_DZJ-731 | 3.97 | 1.74 | 43.37 | 30.67 | 16.87 | 0.06 | 6.74 |
| FTG650411 | M3_DZJ-731 | 3.76 | 2.45 | 49.43 | 28.53 | 12.71 | 0.04 | 7.28 |
| FTG650421 | M3_DZJ-731 | 3.92 | 2.64 | 47.33 | 29.77 | 12.98 | 0.06 | 7.80 |
| FTG650423 | M3_DZJ-731 | 3.87 | 2.22 | 46.08 | 30.36 | 14.06 | 0.07 | 7.24 |
| FTG650431 | M3_DZJ-731 | 3.69 | 2.42 | 48.95 | 29.05 | 12.71 | 0.04 | 7.25 |
| FTG650381 | M3_DZJ-751 | 4.02 | 2.06 | 45.72 | 29.51 | 15.41 | 0.05 | 7.13 |
| FTG650384 | M3_DZJ-751 | 4.20 | 1.91 | 42.68 | 27.52 | 20.57 | 0.05 | 7.04 |
| FTG650390 | M3_DZJ-751 | 4.02 | 2.22 | 45.04 | 26.69 | 18.74 | 0.04 | 7.29 |
| FTG650391 | M3_DZJ-751 | 4.52 | 2.01 | 46.43 | 29.40 | 14.50 | 0.05 | 7.59 |
| FTG650399 | M3_DZJ-751 | 4.32 | 2.36 | 40.73 | 30.20 | 18.77 | 0.05 | 7.93 |
| FTG650405 | M3_DZJ-751 | 4.24 | 2.19 | 43.91 | 32.16 | 14.09 | 0.06 | 7.59 |
| FTG650409 | M3_DZJ-751 | 4.10 | 2.35 | 45.46 | 28.80 | 16.01 | 0.05 | 7.54 |
| FTG650375 | M3_DZJ-793 | 4.26 | 2.51 | 48.04 | 29.88 | 11.78 | 0.04 | 8.03 |
| FTG650377 | M3_DZJ-793 | 4.58 | 2.03 | 45.81 | 29.72 | 14.48 | 0.03 | 7.78 |
| FTG650388 | M3_DZJ-793 | 4.30 | 2.35 | 49.55 | 28.62 | 11.85 | 0.04 | 7.82 |
| FTG650389 | M3_DZJ-793 | 4.39 | 2.41 | 48.72 | 29.16 | 12.08 | 0.05 | 7.94 |
| FTG650397 | M3_DZJ-793 | 4.56 | 2.47 | 48.20 | 29.77 | 11.73 | 0.04 | 8.23 |
| FTG650400 | M3_DZJ-793 | 4.43 | 2.28 | 47.45 | 30.16 | 12.27 | 0.05 | 7.88 |
| FTG650404 | M3_DZJ-793 | 4.37 | 2.27 | 48.30 | 29.59 | 12.21 | 0.05 | 7.77 |
| FTG650432 | M3_DZJ-793 | 4.27 | 2.42 | 50.46 | 28.12 | 11.45 | 0.05 | 7.87 |
| FTG650365 | M3_DZJ-816 | 3.67 | 2.23 | 55.69 | 25.04 | 10.13 | 0.04 | 7.03 |
| FTG650395 | M3_DZJ-816 | 3.61 | 2.80 | 55.18 | 25.51 | 9.64 | 0.03 | 7.62 |
| FTG650402 | M3_DZJ-816 | 3.74 | 2.56 | 55.68 | 25.50 | 9.46 | 0.03 | 7.39 |
| FTG650410 | M3_DZJ-816 | 3.74 | 2.68 | 55.27 | 25.44 | 9.76 | 0.04 | 7.56 |
| FTG650415 | M3_DZJ-816 | 3.54 | 3.15 | 55.00 | 25.64 | 9.35 | 0.04 | 7.98 |
| FTG650422 | M3_DZJ-816 | 3.57 | 2.85 | 55.70 | 24.61 | 9.95 | 0.06 | 7.70 |
| FTG650430 | M3_DZJ-816 | 3.63 | 2.68 | 54.87 | 25.55 | 10.02 | 0.03 | 7.56 |
| FTG650548 | M3_DZJ-857 | 5.03 | 2.17 | 43.18 | 30.55 | 15.60 | 0.06 | 8.38 |
| FTG650549 | M3_DZJ-857 | 4.55 | 3.07 | 40.74 | 31.12 | 16.73 | 0.06 | 9.10 |
| FTG650550 | M3_DZJ-857 | 4.70 | 2.42 | 45.67 | 29.68 | 14.10 | 0.06 | 8.40 |
| FTG650603 | M3_DZJ-857 | 4.58 | 1.95 | 46.04 | 29.17 | 14.58 | 0.06 | 7.55 |
| FTG650609 | M3_DZJ-857 | 4.64 | 1.97 | 44.21 | 30.31 | 15.30 | 0.04 | 7.77 |
| FTG650613 | M3_DZJ-857 | 4.75 | 2.71 | 43.28 | 29.61 | 15.91 | 0.04 | 8.79 |
| FTG650652 | M3_DZJ-857 | 4.69 | 2.27 | 43.39 | 31.01 | 15.22 | 0.05 | 8.13 |
| FTG650655 | M3_DZJ-857 | 4.95 | 2.13 | 44.14 | 30.33 | 15.02 | 0.05 | 8.20 |
| FTG650656 | M3_DZJ-857 | 4.95 | 2.66 | 42.75 | 30.53 | 15.48 | 0.13 | 8.91 |
| FTG650660 | M3_DZJ-857 | 4.95 | 1.95 | 41.57 | 31.37 | 16.67 | 0.06 | 8.08 |

### Example 4

### Gamma Radiation Mutagenesis

A line designated rrm1367-003 displaying an increased 18:3 fatty acid content in the seed oil fraction of greater than 16% was developed by two rounds of gamma radiation mutagenesis of seeds starting with the parent line B. napus cv. Topas. In the first round of mutagenesis the seeds were exposed to 40,000 Rads of gamma radiation and subsequently designated “M₁” seed. Plant from the M₁ seed were grown and allowed to cross pollinate. Seed from the M₁ plants, which showed little if any effects from the radiation treatment, were collected. A portion of the seed from the M₁ plants was subjected to 60,000 rads of gamma radiation and subsequently designated M₂ seed. M₂ seed was sown and individual plants bagged to prevent cross pollination. The content of the seed oil fraction of individual plant was assessed by gas chromatography as described in Example 1. From the plants grown from the M₂ seed plants were selected and selfed three times to develop lines from which rrm1367-003 for its seed oil phenotype.

### Example 5

### Mapping of the QTLs for Elevated Seed Oil 18:3 Fatty Acid Levels in B. napus Rrm1367-003

An F2 population was developed by crossing rrm1367-003 and the elite breeding line RO011. Using 13,997 Single Nucleotide Polymorphisms (SNPs) markers to genotype 173 F2 plants two genomic blocks on B. napus chromosomes N12 and N17 were identified to be significantly associated with C18:3 content (R-values: 0.74 and 0.52, respectively). The QTL on N12 accounts for the majority of the phenotypic variance on C18:3 content. FIG. 1 shows a genetic linkage map of N12, including SNP markers defining the genomic blocks conferring elevated 18:3 content in rrm1367-003. A list of some SNPs on chromosome 12 associate with elevated 18:3 fatty acid content can be found in Table 1 and Table 2. FIG. 2 shows a genetic linkage map of N17, including SNP markers defining the genomic blocks conferring elevated 18:3 content in rrm1367-003. A list of some SNPs on chromosome 17 associate with elevated 18:3 fatty acid content can be found in Table 3.

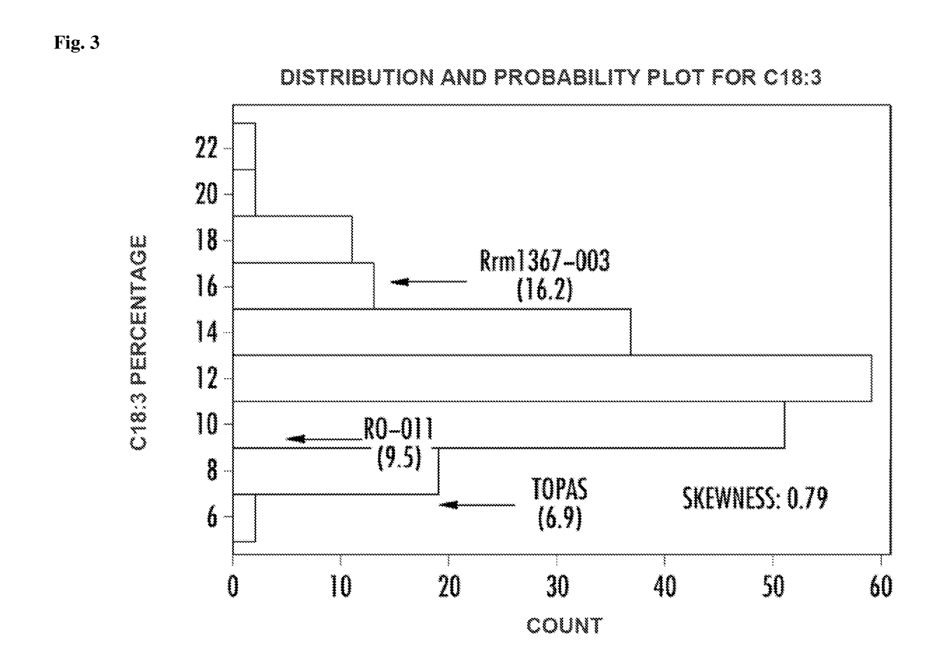

The oil components of seeds from 196 plants of the F2 population, its two crossing parental lines (rrm1367-003 and the elite breeding line RO011) and the low linolenic acid producing line “Topas” were analyzed (Table 8). The distribution of C18:3 fatty acid content of the seed oil from those F2 plants, along with the C18:3 content of rrm1367-003, RO011 and Topas is shown in FIG. 3. In addition to the elevated C18:3 fatty acid content of some F2 individuals, the plants demonstrate a negative correlation between C18:1 and C18:2 (R-value: −0.74) and between C18:1 and C18:3 (R-value: −0.77) and transgressive segregation of C18:3 content in the F2 population. Table 7 shows the results of Pearson correlation analysis among the oil components using 196 F2 plants, and analysis of the oil fraction of seeds from individual plants is shown in Table 8 below.

TABLE 7
Pearson Correlation Coefficients

| | C16:0 | C18:0 | C18:1 | C18:2 | C18:3 | Sats |
|---|---|---|---|---|---|---|
| C16:0 | 1 | −0.25631 | −0.64647 | 0.74038 | 0.17487 | 0.62193 |
| | | 0.0003 | <.0001 | <.0001 | 0.0142 | <.0001 |
| C18:0 | −0.25631 | 1 | −0.02447 | −0.31602 | 0.20026 | 0.53362 |
| | 0.0003 | | 0.7335 | <.0001 | 0.0049 | <.0001 |
| C18:1 | −0.64647 | −0.02447 | 1 | −0.77485 | −0.7508 | −0.57068 |
| | <.0001 | 0.7335 | | <.0001 | <.0001 | <.0001 |
| C18:2 | 0.74038 | −0.31602 | −0.77485 | 1 | 0.20384 | 0.39726 |
| | <.0001 | <.0001 | <.0001 | | 0.0042 | <.0001 |
| C18:3 | 0.17487 | 0.20026 | −0.7508 | 0.20384 | 1 | 0.27265 |
| | 0.0142 | 0.0049 | <.0001 | 0.0042 | | 0.0001 |
| Sats | 0.62193 | 0.53362 | −0.57068 | 0.39726 | 0.27265 | 1 |
| | <.0001 | <.0001 | <.0001 | <.0001 | <0.0001 | |

N = 196, Prob > | r | under H0: Rho = 0

TABLE 8
Fatty Acid Profile of F2 Plants

| SAMPLE ID | RESCHID | C14:0 | C16:0 | C16:1 | C18:0 | C18:1 | C18:2 | C18:3 |
|---|---|---|---|---|---|---|---|---|
| FTJ625705 | RO11_1367r | 0.00 | 5.75 | 0.40 | 3.16 | 46.09 | 18.95 | 22.30 |
| FTJ625740 | RO11_1367r | 0.05 | 5.44 | 0.37 | 3.30 | 44.50 | 21.70 | 21.06 |
| FTJ625559 | RO11_1367r | 0.10 | 4.73 | 0.30 | 2.00 | 51.29 | 18.78 | 20.48 |
| FTJ625616 | RO11_1367r | 0.05 | 5.16 | 0.33 | 2.71 | 49.03 | 20.76 | 19.18 |
| FTJ625632 | RO11_1367r | 0.05 | 4.67 | 0.30 | 3.23 | 52.47 | 17.98 | 18.56 |
| FTJ625518 | RO11_1367r | 0.04 | 4.52 | 0.29 | 2.98 | 53.79 | 17.77 | 18.43 |
| FTJ625543 | RO11_1367r | 0.11 | 3.80 | 0.22 | 3.41 | 55.77 | 15.19 | 18.40 |
| FTJ625717 | RO11_1367r | 0.00 | 4.38 | 0.25 | 2.58 | 52.81 | 18.47 | 18.25 |
| FTJ625669 | RO11_1367r | 0.07 | 5.47 | 0.43 | 2.14 | 48.46 | 22.06 | 17.84 |
| FTJ625721 | RO11_1367r | 0.03 | 4.00 | 0.22 | 2.83 | 55.53 | 16.55 | 17.69 |
| FTJ625644 | RO11_1367r | 0.04 | 4.62 | 0.30 | 2.55 | 53.46 | 18.01 | 17.62 |
| FTJ625612 | RO11_1367r | 0.05 | 5.75 | 0.49 | 2.50 | 45.93 | 23.97 | 17.60 |
| FTJ625752 | RO11_1367r | 0.05 | 5.18 | 0.32 | 3.15 | 51.28 | 19.26 | 17.59 |
| FTJ625720 | RO11_1367r | 0.06 | 4.36 | 0.24 | 3.74 | 52.20 | 18.49 | 17.53 |
| FTJ625628 | RO11_1367r | 0.04 | 4.22 | 0.24 | 3.04 | 56.93 | 15.91 | 16.97 |
| FTJ625653 | RO11_1367r | 0.00 | 4.88 | 0.27 | 2.93 | 51.47 | 19.77 | 16.85 |
| FTJ625542 | RO11_1367r | 0.04 | 4.51 | 0.29 | 3.60 | 55.22 | 17.58 | 16.43 |
| FTJ625742 | RO11_1367r | 0.00 | 7.14 | 0.76 | 2.73 | 41.63 | 28.31 | 16.38 |
| FTJ625536 | RO11_1367r | 0.11 | 4.28 | 0.25 | 2.50 | 59.55 | 15.22 | 16.01 |
| FTJ625519 | RO11_1367r | 0.07 | 3.84 | 0.25 | 2.86 | 56.71 | 17.48 | 15.83 |
| FTJ625647 | RO11_1367r | 0.04 | 4.92 | 0.32 | 2.41 | 53.20 | 20.50 | 15.79 |
| FTJ625532 | RO11_1367r | 0.06 | 3.93 | 0.25 | 2.78 | 58.52 | 16.38 | 15.76 |
| FTJ625530 | RO11_1367r | 0.07 | 4.87 | 0.32 | 2.55 | 54.36 | 20.12 | 15.58 |
| FTJ625723 | RO11_1367r | 0.04 | 4.37 | 0.29 | 2.72 | 56.95 | 16.66 | 15.54 |
| FTJ625585 | RO11_1367r | 0.04 | 4.48 | 0.28 | 3.05 | 55.44 | 18.10 | 15.51 |
| FTJ625626 | RO11_1367r | 0.05 | 4.66 | 0.32 | 2.67 | 54.18 | 18.79 | 15.45 |
| FTJ625548 | RO11_1367r | 0.13 | 4.47 | 0.30 | 3.38 | 55.09 | 18.50 | 15.38 |
| FTJ625634 | RO11_1367r | 0.05 | 5.54 | 0.36 | 2.73 | 50.29 | 22.27 | 15.25 |
| FTJ625664 | RO11_1367r | 0.04 | 4.00 | 0.22 | 4.10 | 57.16 | 15.66 | 14.95 |
| FTJ625526 | RO11_1367r | 0.06 | 5.18 | 0.32 | 2.88 | 52.26 | 21.87 | 14.87 |
| FTJ625633 | RO11_1367r | 0.04 | 4.66 | 0.26 | 2.67 | 53.97 | 20.45 | 14.71 |
| FTJ625726 | RO11_1367r | 0.05 | 5.15 | 0.36 | 2.54 | 49.99 | 24.41 | 14.69 |
| FTJ625525 | RO11_1367r | 0.08 | 5.14 | 0.50 | 1.60 | 48.30 | 27.04 | 14.67 |
| FTJ625695 | RO11_1367r | 0.03 | 3.70 | 0.19 | 3.27 | 58.87 | 15.98 | 14.61 |
| FTJ625649 | RO11_1367r | 0.03 | 4.42 | 0.24 | 3.19 | 55.71 | 18.58 | 14.47 |
| FTJ625641 | RO11_1367r | 0.06 | 4.84 | 0.41 | 1.91 | 53.58 | 21.35 | 14.33 |
| FTJ625690 | RO11_1367r | 0.00 | 3.77 | 0.19 | 3.05 | 59.21 | 15.72 | 14.25 |
| FTJ625535 | RO11_1367r | 0.12 | 5.31 | 0.48 | 2.27 | 49.07 | 25.51 | 14.16 |
| FTJ625605 | RO11_1367r | 0.04 | 4.41 | 0.24 | 3.14 | 56.69 | 17.98 | 14.11 |
| FTJ625524 | RO11_1367r | 0.05 | 4.32 | 0.29 | 1.88 | 58.26 | 18.78 | 14.09 |
| FTJ625718 | RO11_1367r | 0.05 | 4.59 | 0.24 | 3.62 | 56.60 | 17.62 | 14.08 |
| FTJ625689 | RO11_1367r | 0.04 | 4.66 | 0.26 | 2.72 | 57.27 | 17.95 | 14.02 |
| FTJ625678 | RO11_1367r | 0.05 | 4.41 | 0.33 | 3.77 | 58.37 | 15.83 | 13.98 |
| FTJ625688 | RO11_1367r | 0.04 | 4.77 | 0.28 | 2.11 | 58.19 | 17.18 | 13.90 |
| FTJ625643 | RO11_1367r | 0.04 | 4.95 | 0.30 | 2.42 | 54.81 | 20.28 | 13.90 |
| FTJ625735 | RO11_1367r | 0.03 | 4.88 | 0.28 | 2.87 | 54.95 | 19.91 | 13.82 |
| FTJ625703 | RO11_1367r | 0.04 | 4.57 | 0.26 | 2.30 | 59.32 | 16.47 | 13.78 |
| FTJ625615 | RO11_1367r | 0.04 | 4.53 | 0.29 | 3.15 | 55.08 | 19.99 | 13.77 |
| FTJ625598 | RO11_1367r | 0.03 | 4.19 | 0.24 | 3.41 | 59.37 | 16.26 | 13.71 |
| FTJ625696 | RO11_1367r | 0.04 | 4.57 | 0.26 | 3.06 | 56.21 | 18.98 | 13.71 |
| FTJ625670 | RO11_1367r | 0.06 | 5.16 | 0.36 | 2.61 | 55.21 | 20.08 | 13.66 |
| FTJ625576 | RO11_1367r | 0.05 | 4.41 | 0.30 | 2.62 | 54.11 | 21.22 | 13.65 |
| FTJ625732 | RO11_1367r | 0.04 | 4.27 | 0.26 | 2.70 | 57.23 | 18.41 | 13.62 |
| FTJ625710 | RO11_1367r | 0.04 | 3.99 | 0.21 | 3.20 | 58.55 | 17.72 | 13.59 |
| FTJ625578 | RO11_1367r | 0.04 | 4.41 | 0.27 | 3.00 | 57.81 | 17.38 | 13.58 |
| FTJ625668 | RO11_1367r | 0.00 | 3.61 | 0.23 | 2.96 | 58.86 | 17.22 | 13.52 |
| FTJ625599 | RO11_1367r | 0.04 | 4.22 | 0.25 | 2.52 | 60.45 | 16.25 | 13.43 |
| FTJ625709 | RO11_1367r | 0.00 | 4.69 | 0.38 | 2.75 | 54.41 | 20.27 | 13.37 |
| FTJ625520 | RO11_1367r | 0.12 | 4.81 | 0.32 | 2.50 | 60.22 | 16.98 | 13.12 |
| FTJ625523 | RO11_1367r | 0.07 | 4.57 | 0.37 | 2.13 | 54.75 | 21.99 | 13.11 |
| FTJ625682 | RO11_1367r | 0.04 | 4.37 | 0.28 | 2.74 | 58.92 | 16.92 | 13.06 |
| FTJ625749 | RO11_1367r | 0.04 | 3.62 | 0.20 | 3.23 | 59.78 | 16.74 | 13.05 |
| FTJ625640 | RO11_1367r | 0.04 | 4.71 | 0.33 | 3.36 | 56.68 | 19.16 | 13.01 |
| FTJ625521 | RO11_1367r | 0.05 | 4.67 | 0.30 | 2.16 | 59.46 | 17.48 | 12.98 |
| FTJ625636 | RO11_1367r | 0.04 | 3.91 | 0.25 | 3.11 | 60.61 | 16.28 | 12.97 |
| FTJ625592 | RO11_1367r | 0.05 | 4.17 | 0.27 | 2.18 | 61.53 | 15.59 | 12.92 |
| FTJ625584 | RO11_1367r | 0.04 | 4.14 | 0.27 | 2.75 | 62.51 | 14.53 | 12.92 |
| FTJ625645 | RO11_1367r | 0.00 | 3.70 | 0.19 | 3.37 | 60.41 | 16.08 | 12.84 |
| FTJ625691 | RO11_1367r | 0.04 | 5.19 | 0.34 | 3.07 | 58.11 | 17.71 | 12.83 |
| FTJ625648 | RO11_1367r | 0.04 | 4.58 | 0.30 | 2.46 | 60.87 | 16.29 | 12.79 |
| FTJ625753 | RO11_1367r | 0.04 | 4.80 | 0.34 | 3.44 | 55.76 | 19.53 | 12.76 |
| FTJ625603 | RO11_1367r | 0.05 | 4.22 | 0.29 | 2.94 | 60.10 | 16.59 | 12.70 |
| FTJ625692 | RO11_1367r | 0.04 | 3.89 | 0.22 | 3.80 | 58.34 | 17.96 | 12.68 |
| FTJ625727 | RO11_1367r | 0.04 | 4.33 | 0.27 | 2.35 | 57.36 | 19.69 | 12.65 |
| FTJ625556 | RO11_1367r | 0.10 | 4.68 | 0.41 | 2.10 | 57.29 | 20.48 | 12.65 |
| FTJ625660 | RO11_1367r | 0.05 | 5.57 | 0.47 | 2.14 | 54.28 | 21.84 | 12.63 |
| FTJ625575 | RO11_1367r | 0.04 | 5.27 | 0.38 | 2.82 | 58.94 | 16.80 | 12.60 |
| FTJ625731 | RO11_1367r | 0.05 | 4.66 | 0.26 | 3.03 | 60.19 | 16.55 | 12.59 |
| FTJ625533 | RO11_1367r | 0.07 | 4.79 | 0.33 | 2.09 | 57.21 | 20.38 | 12.59 |
| FTJ625725 | RO11_1367r | 0.05 | 4.48 | 0.28 | 3.17 | 56.81 | 19.64 | 12.53 |
| FTJ625683 | RO11_1367r | 0.04 | 3.62 | 0.18 | 3.84 | 59.68 | 16.97 | 12.53 |
| FTJ625541 | RO11_1367r | 0.06 | 5.21 | 0.45 | 2.19 | 51.26 | 25.47 | 12.50 |
| FTJ625586 | RO11_1367r | 0.06 | 5.26 | 0.37 | 2.48 | 53.93 | 22.61 | 12.44 |
| FTJ625712 | RO11_1367r | 0.04 | 4.15 | 0.21 | 3.47 | 57.89 | 18.32 | 12.42 |
| FTJ625589 | RO11_1367r | 0.05 | 4.48 | 0.35 | 3.35 | 60.07 | 16.17 | 12.33 |
| FTJ625666 | RO11_1367r | 0.03 | 5.37 | 0.35 | 3.99 | 54.73 | 19.81 | 12.23 |
| FTJ625614 | RO11_1367r | 0.05 | 4.03 | 0.23 | 4.20 | 61.25 | 15.35 | 12.08 |
| FTJ625657 | RO11_1367r | 0.00 | 4.48 | 0.25 | 2.03 | 60.27 | 17.92 | 12.06 |
| FTJ625734 | RO11_1367r | 0.06 | 5.54 | 0.45 | 2.46 | 51.76 | 22.56 | 11.99 |
| FTJ625681 | RO11_1367r | 0.05 | 5.23 | 0.58 | 2.43 | 54.41 | 20.98 | 11.92 |
| FTJ625676 | RO11_1367r | 0.04 | 4.91 | 0.35 | 2.36 | 57.49 | 19.77 | 11.90 |
| FTJ625639 | RO11_1367r | 0.04 | 3.83 | 0.23 | 3.03 | 63.01 | 14.89 | 11.88 |
| FTJ625522 | RO11_1367r | 0.07 | 4.58 | 0.30 | 2.76 | 56.31 | 21.18 | 11.87 |
| FTJ625528 | RO11_1367r | 0.07 | 4.91 | 0.38 | 2.25 | 54.75 | 22.92 | 11.86 |
| FTJ625568 | RO11_1367r | 0.06 | 4.37 | 0.29 | 2.56 | 60.51 | 18.23 | 11.85 |
| FTJ625674 | RO11_1367r | 0.04 | 4.12 | 0.25 | 2.04 | 57.98 | 20.49 | 11.81 |
| FTJ625606 | RO11_1367r | 0.04 | 4.74 | 0.28 | 2.28 | 57.76 | 19.66 | 11.73 |
| FTJ625604 | RO11_1367r | 0.05 | 4.13 | 0.24 | 2.75 | 65.17 | 13.34 | 11.68 |
| FTJ625686 | RO11_1367r | 0.00 | 4.74 | 0.29 | 2.91 | 57.55 | 19.43 | 11.66 |
| FTJ625713 | RO11_1367r | 0.03 | 4.80 | 0.34 | 2.11 | 60.73 | 17.23 | 11.66 |
| FTJ625693 | RO11_1367r | 0.03 | 4.64 | 0.27 | 2.98 | 58.69 | 18.56 | 11.65 |
| FTJ625741 | RO11_1367r | 0.06 | 4.55 | 0.36 | 3.44 | 57.92 | 18.11 | 11.63 |
| FTJ625620 | RO11_1367r | 0.14 | 4.74 | 0.37 | 2.39 | 57.69 | 20.52 | 11.62 |
| FTJ625537 | RO11_1367r | 0.10 | 4.30 | 0.26 | 2.25 | 61.63 | 17.34 | 11.61 |
| FTJ625663 | RO11_1367r | 0.04 | 4.30 | 0.27 | 3.12 | 60.59 | 16.98 | 11.56 |
| FTJ625677 | RO11_1367r | 0.05 | 4.28 | 0.27 | 2.44 | 59.54 | 18.88 | 11.51 |
| FTJ625527 | RO11_1367r | 0.07 | 4.79 | 0.42 | 2.16 | 62.49 | 16.04 | 11.50 |
| FTJ625635 | RO11_1367r | 0.05 | 4.36 | 0.35 | 2.40 | 58.54 | 19.57 | 11.49 |
| FTJ625724 | RO11_1367r | 0.06 | 5.38 | 0.45 | 2.36 | 53.45 | 23.58 | 11.48 |
| FTJ625722 | RO11_1367r | 0.04 | 4.91 | 0.36 | 1.99 | 57.51 | 20.69 | 11.45 |
| FTJ625680 | RO11_1367r | 0.04 | 4.02 | 0.23 | 2.80 | 62.56 | 15.72 | 11.42 |
| FTJ625675 | RO11_1367r | 0.03 | 4.49 | 0.26 | 2.63 | 60.94 | 17.31 | 11.39 |
| FTJ625698 | RO11_1367r | 0.04 | 3.44 | 0.17 | 3.32 | 60.48 | 17.25 | 11.39 |
| FTJ625596 | RO11_1367r | 0.04 | 3.42 | 0.19 | 2.58 | 64.64 | 14.60 | 11.37 |
| FTJ625714 | RO11_1367r | 0.04 | 4.09 | 0.22 | 2.69 | 63.28 | 14.80 | 11.36 |
| FTJ625569 | RO11_1367r | 0.08 | 3.94 | 0.24 | 2.41 | 61.91 | 17.36 | 11.26 |
| FTJ625667 | RO11_1367r | 0.05 | 4.73 | 0.32 | 1.94 | 60.34 | 18.82 | 11.26 |
| FTJ625697 | RO11_1367r | 0.06 | 5.25 | 0.47 | 2.77 | 51.97 | 22.95 | 11.16 |
| FTJ625748 | RO11_1367r | 0.03 | 3.97 | 0.21 | 3.44 | 62.95 | 14.99 | 11.13 |
| FTJ625613 | RO11_1367r | 0.04 | 3.83 | 0.22 | 2.77 | 62.68 | 16.82 | 11.07 |
| FTJ625672 | RO11_1367r | 0.04 | 4.50 | 0.30 | 2.35 | 61.71 | 16.74 | 11.03 |
| FTJ625595 | RO11_1367r | 0.04 | 4.32 | 0.26 | 2.40 | 63.57 | 15.83 | 11.01 |
| FTJ625646 | RO11_1367r | 0.05 | 6.20 | 0.48 | 2.41 | 56.77 | 19.76 | 10.98 |
| FTJ625746 | RO11_1367r | 0.04 | 3.94 | 0.24 | 3.09 | 62.53 | 16.29 | 10.97 |
| FTJ625706 | RO11_1367r | 0.05 | 6.38 | 0.66 | 2.62 | 53.71 | 22.12 | 10.94 |
| FTJ625594 | RO11_1367r | 0.22 | 5.52 | 0.48 | 2.65 | 54.64 | 22.08 | 10.94 |
| FTJ625704 | RO11_1367r | 0.04 | 4.13 | 0.25 | 2.18 | 63.21 | 16.51 | 10.94 |
| FTJ625684 | RO11_1367r | 0.04 | 4.41 | 0.29 | 2.55 | 61.10 | 17.37 | 10.90 |
| FTJ625627 | RO11_1367r | 0.00 | 4.10 | 0.22 | 2.70 | 63.94 | 14.97 | 10.89 |
| FTJ625564 | RO11_1367r | 0.06 | 4.36 | 0.29 | 2.24 | 58.54 | 20.67 | 10.85 |
| FTJ625652 | RO11_1367r | 0.03 | 4.63 | 0.25 | 3.74 | 59.72 | 17.66 | 10.83 |
| FTJ625659 | RO11_1367r | 0.04 | 4.58 | 0.28 | 2.66 | 63.54 | 15.51 | 10.82 |
| FTJ625608 | RO11_1367r | 0.06 | 4.67 | 0.30 | 2.81 | 62.18 | 16.35 | 10.73 |
| FTJ625621 | RO11_1367r | 0.08 | 4.26 | 0.28 | 1.91 | 62.48 | 17.66 | 10.62 |
| FTJ625557 | RO11_1367r | 0.12 | 5.99 | 0.58 | 1.94 | 54.80 | 23.18 | 10.61 |
| FTJ625661 | RO11_1367r | 0.06 | 4.57 | 0.31 | 2.45 | 62.44 | 16.07 | 10.55 |
| FTJ625551 | RO11_1367r | 0.09 | 4.34 | 0.29 | 1.85 | 60.45 | 19.96 | 10.53 |
| FTJ625538 | RO11_1367r | 0.12 | 3.91 | 0.24 | 2.78 | 64.92 | 15.46 | 10.51 |
| FTJ625656 | RO11_1367r | 0.04 | 4.86 | 0.33 | 3.03 | 58.78 | 19.17 | 10.39 |
| FTJ625618 | RO11_1367r | 0.04 | 4.22 | 0.25 | 2.86 | 61.52 | 17.84 | 10.38 |
| FTJ625671 | RO11_1367r | 0.04 | 4.49 | 0.27 | 2.49 | 61.16 | 18.11 | 10.32 |
| FTJ625679 | RO11_1367r | 0.06 | 4.75 | 0.35 | 2.81 | 61.42 | 17.28 | 10.20 |
| FTJ625562 | RO11_1367r | 0.20 | 4.99 | 0.46 | 1.87 | 57.70 | 22.49 | 10.20 |
| FTJ625716 | RO11_1367r | 0.06 | 5.13 | 0.37 | 2.63 | 63.04 | 15.44 | 10.17 |
| FTJ625673 | RO11_1367r | 0.04 | 3.91 | 0.24 | 3.31 | 64.18 | 15.13 | 10.17 |
| FTJ625655 | RO11_1367r | 0.04 | 4.33 | 0.26 | 3.06 | 62.76 | 16.23 | 10.15 |
| FTJ625650 | RO11_1367r | 0.05 | 4.39 | 0.28 | 2.12 | 62.50 | 17.93 | 10.12 |
| FTJ625651 | RO11_1367r | 0.03 | 4.60 | 0.27 | 2.93 | 61.77 | 16.73 | 10.08 |
| FTJ625567 | RO11_1367r | 0.06 | 4.13 | 0.30 | 2.77 | 64.31 | 16.15 | 10.05 |
| FTJ625654 | RO11_1367r | 0.00 | 3.88 | 0.19 | 2.61 | 63.59 | 16.03 | 9.94 |
| FTJ625699 | RO11_1367r | 0.06 | 5.71 | 0.46 | 1.87 | 54.18 | 24.99 | 9.94 |
| FTJ625719 | RO11_1367r | 0.00 | 3.67 | 0.21 | 3.02 | 64.93 | 15.36 | 9.93 |
| FTJ625573 | RO11_1367r | 0.05 | 4.61 | 0.35 | 2.14 | 58.65 | 20.71 | 9.88 |
| FTJ625700 | RO11_1367r | 0.04 | 4.17 | 0.26 | 2.55 | 66.00 | 14.02 | 9.85 |
| FTJ625685 | RO11_1367r | 0.04 | 4.53 | 0.30 | 2.53 | 63.48 | 16.02 | 9.83 |
| FTJ625546 | RO11_1367r | 0.07 | 4.15 | 0.28 | 2.63 | 63.48 | 16.49 | 9.83 |
| FTJ625715 | RO11_1367r | 0.00 | 5.22 | 0.40 | 3.04 | 51.71 | 24.89 | 9.79 |
| FTJ625750 | RO11_1367r | 0.04 | 3.80 | 0.22 | 2.48 | 65.98 | 14.97 | 9.77 |
| FTJ625745 | RO11_1367r | 0.04 | 4.57 | 0.30 | 2.48 | 62.93 | 17.46 | 9.75 |
| FTJ625600 | RO11_1367r | 0.04 | 4.06 | 0.24 | 2.62 | 63.62 | 16.50 | 9.74 |
| FTJ625658 | RO11_1367r | 0.00 | 4.13 | 0.26 | 2.64 | 64.94 | 14.71 | 9.73 |
| FTJ625665 | RO11_1367r | 0.06 | 4.68 | 0.30 | 2.88 | 62.51 | 16.50 | 9.69 |
| FTJ625708 | RO11_1367r | 0.04 | 3.91 | 0.20 | 2.49 | 62.83 | 17.15 | 9.69 |
| FTJ625743 | RO11_1367r | 0.04 | 4.16 | 0.28 | 2.60 | 63.13 | 17.12 | 9.68 |
| FTJ625619 | RO11_1367r | 0.04 | 4.47 | 0.27 | 3.18 | 62.50 | 16.88 | 9.67 |
| FTJ625730 | RO11_1367r | 0.04 | 4.83 | 0.32 | 2.39 | 62.17 | 17.51 | 9.51 |
| FTJ625597 | RO11_1367r | 0.04 | 4.06 | 0.25 | 2.24 | 64.13 | 16.71 | 9.27 |
| FTJ625571 | RO11_1367r | 0.06 | 4.93 | 0.42 | 2.11 | 58.21 | 22.41 | 9.24 |
| FTJ625609 | RO11_1367r | 0.05 | 4.50 | 0.26 | 2.47 | 63.63 | 16.72 | 9.23 |
| FTJ625588 | RO11_1367r | 0.04 | 4.35 | 0.30 | 2.36 | 64.77 | 16.10 | 9.18 |
| FTJ625739 | RO11_1367r | 0.04 | 4.10 | 0.26 | 2.14 | 63.83 | 16.55 | 9.13 |
| FTJ625701 | RO11_1367r | 0.04 | 3.95 | 0.24 | 2.54 | 66.20 | 15.04 | 9.06 |
| FTJ625602 | RO11_1367r | 0.05 | 4.74 | 0.29 | 2.37 | 63.04 | 17.41 | 9.05 |
| FTJ625707 | RO11_1367r | 0.04 | 4.79 | 0.29 | 2.23 | 62.48 | 18.03 | 9.02 |
| FTJ625554 | RO11_1367r | 0.08 | 4.03 | 0.25 | 3.03 | 64.88 | 16.17 | 9.00 |
| FTJ625610 | RO11_1367r | 0.03 | 4.06 | 0.21 | 3.08 | 65.76 | 14.79 | 8.84 |
| FTJ625733 | RO11_1367r | 0.04 | 4.16 | 0.25 | 2.70 | 62.43 | 18.31 | 8.74 |
| FTJ625736 | RO11_1367r | 0.00 | 5.51 | 0.40 | 2.47 | 53.74 | 23.88 | 8.72 |
| FTJ625694 | RO11_1367r | 0.06 | 5.00 | 0.37 | 2.67 | 61.57 | 18.84 | 8.68 |
| FTJ625747 | RO11_1367r | 0.04 | 4.04 | 0.22 | 3.33 | 63.59 | 16.81 | 8.67 |
| FTJ625642 | RO11_1367r | 0.06 | 5.44 | 0.62 | 3.04 | 56.70 | 21.95 | 8.63 |
| FTJ625558 | RO11_1367r | 0.14 | 5.76 | 0.49 | 2.35 | 60.19 | 20.30 | 8.57 |
| FTJ625687 | RO11_1367r | 0.03 | 4.26 | 0.22 | 2.74 | 64.82 | 16.27 | 8.56 |
| FTJ625534 | RO11_1367r | 0.04 | 4.70 | 0.34 | 2.66 | 62.76 | 18.76 | 8.48 |
| FTJ625611 | RO11_1367r | 0.07 | 5.39 | 0.44 | 2.53 | 60.25 | 18.99 | 8.31 |
| FTJ625729 | RO11_1367r | 0.00 | 3.77 | 0.20 | 2.66 | 66.42 | 15.45 | 8.25 |
| FTJ625590 | RO11_1367r | 0.05 | 4.41 | 0.25 | 2.76 | 65.84 | 15.32 | 7.99 |
| FTJ625662 | RO11_1367r | 0.04 | 4.47 | 0.27 | 2.67 | 61.75 | 19.18 | 7.99 |
| FTJ625582 | RO11_1367r | 0.06 | 5.01 | 0.38 | 3.04 | 63.85 | 16.66 | 7.91 |
| FTJ625638 | RO11_1367r | 0.00 | 4.24 | 0.25 | 2.12 | 63.45 | 18.75 | 7.90 |
| FTJ625702 | RO11_1367r | 0.05 | 4.05 | 0.23 | 3.16 | 64.54 | 16.49 | 7.80 |
| FTJ625711 | RO11_1367r | 0.05 | 4.28 | 0.29 | 2.73 | 62.40 | 18.35 | 7.61 |
| FTJ625570 | RO11_1367r | 0.07 | 4.28 | 0.33 | 2.44 | 65.13 | 17.64 | 7.61 |
| FTJ625637 | RO11_1367r | 0.06 | 4.24 | 0.27 | 2.81 | 63.55 | 18.91 | 7.52 |
| FTJ625540 | RO11_1367r | 0.04 | 4.36 | 0.25 | 2.50 | 68.00 | 15.53 | 6.69 |
| FTJ625623 | RO11_1367r | 0.04 | 3.93 | 0.22 | 2.32 | 69.07 | 15.68 | 6.31 |
| FTJ625566 | RO011 | 0.07 | 4.64 | 0.31 | 1.91 | 61.00 | 20.27 | 9.98 |
| FTJ625561 | RO011 | 0.07 | 4.74 | 0.30 | 2.29 | 62.06 | 21.11 | 7.21 |
| FTJ625550 | RO011 | 0.17 | 5.21 | 0.35 | 2.36 | 52.52 | 24.23 | 12.97 |
| FTJ625552 | RO011 | 0.11 | 5.34 | 0.41 | 2.46 | 55.20 | 24.10 | 8.94 |
| FTJ625555 | RO011 | 0.13 | 4.94 | 0.34 | 1.95 | 61.09 | 20.69 | 8.66 |
| FTJ625544 | RO011 | 0.10 | 6.65 | 0.62 | 2.29 | 44.54 | 30.82 | 12.63 |
| FTJ625572 | RO011 | 0.09 | 4.88 | 0.32 | 2.27 | 65.71 | 18.37 | 6.48 |
| FTJ625553 | RO011 | 0.08 | 5.36 | 0.38 | 2.55 | 51.57 | 24.67 | 13.07 |
| FTJ625574 | RO011 | 0.07 | 5.83 | 0.41 | 2.12 | 58.45 | 22.81 | 7.53 |
| FTJ625593 | RO011 | 0.07 | 4.87 | 0.31 | 2.12 | 62.94 | 19.50 | 7.80 |
| FTJ625563 | Topas | 0.07 | 4.19 | 0.30 | 1.99 | 65.95 | 16.86 | 7.64 |
| FTJ625549 | Topas | 0.06 | 4.25 | 0.32 | 2.24 | 65.92 | 16.98 | 7.15 |
| FTJ625624 | Topas | 0.03 | 4.03 | 0.26 | 2.35 | 67.91 | 15.17 | 6.66 |
| FTJ625577 | Topas | 0.05 | 4.56 | 0.40 | 3.02 | 55.54 | 21.69 | 10.76 |
| FTJ625625 | Topas | 0.04 | 4.27 | 0.30 | 2.46 | 66.08 | 16.31 | 6.92 |
| FTJ625631 | Topas | 0.03 | 4.16 | 0.28 | 2.35 | 67.36 | 15.45 | 6.69 |
| FTJ625630 | Topas | 0.03 | 4.30 | 0.28 | 2.27 | 67.33 | 15.62 | 6.43 |
| FTJ625629 | Topas | 0.03 | 4.04 | 0.27 | 2.39 | 66.83 | 15.94 | 6.96 |
| FTJ625583 | Topas | 0.00 | 3.91 | 0.24 | 2.35 | 67.69 | 15.25 | 6.66 |
| FTJ625622 | rrm1367-003 | 0.03 | 4.36 | 0.25 | 3.13 | 55.18 | 17.77 | 15.55 |
| FTJ625591 | rrm1367-003 | 0.03 | 4.23 | 0.23 | 2.83 | 55.84 | 17.29 | 15.64 |
| FTJ625601 | rrm1367-003 | 0.03 | 3.97 | 0.21 | 2.94 | 55.33 | 16.84 | 16.88 |
| FTJ625580 | rrm1367-003 | 0.03 | 4.13 | 0.24 | 2.88 | 54.94 | 17.62 | 16.30 |
| FTJ625581 | rrm1367-003 | 0.04 | 4.00 | 0.28 | 2.48 | 49.99 | 20.14 | 19.18 |
| FTJ625587 | rrm1367-003 | 0.03 | 4.36 | 0.24 | 3.08 | 54.62 | 17.68 | 15.84 |
| FTJ625607 | rrm1367-003 | 0.03 | 4.18 | 0.26 | 3.42 | 55.18 | 17.27 | 15.54 |
| FTJ625579 | rrm1367-003 | 0.03 | 4.12 | 0.24 | 2.87 | 55.13 | 17.67 | 15.90 |
| FTJ625617 | rrm1367-003 | 0.03 | 4.14 | 0.22 | 3.08 | 57.04 | 16.81 | 14.90 |
| FTJ625728 | rrm1367-003 | 0.00 | 4.16 | 0.29 | 2.44 | 53.52 | 18.70 | 16.96 |
| FTJ625751 | rrm1367-003 | 0.02 | 4.15 | 0.21 | 3.07 | 56.01 | 17.13 | 15.50 |
| FTJ625737 | rrm1367-003 | 0.03 | 3.95 | 0.19 | 3.05 | 56.45 | 17.19 | 15.07 |
| FTJ625738 | rrm1367-003 | 0.04 | 4.32 | 0.30 | 2.21 | 52.84 | 19.98 | 16.48 |
| FTJ625539 | rrm1367-003 | 0.06 | 4.23 | 0.26 | 2.77 | 55.14 | 17.78 | 16.62 |
| FTJ625565 | rrm1367-003 | 0.04 | 4.23 | 0.27 | 2.96 | 55.72 | 17.39 | 16.18 |
| FTJ625560 | rrm1367-003 | 0.04 | 4.09 | 0.26 | 2.84 | 55.02 | 17.91 | 16.67 |
| FTJ625531 | rrm1367-003 | 0.04 | 4.20 | 0.26 | 3.05 | 55.29 | 17.74 | 16.08 |
| FTJ625529 | rrm1367-003 | 0.04 | 4.07 | 0.24 | 2.94 | 55.76 | 17.48 | 16.08 |
| FTJ625547 | rrm1367-003 | 0.03 | 4.22 | 0.26 | 2.88 | 54.21 | 18.35 | 16.64 |
| FTJ625545 | rrm1367-003 | 0.06 | 4.12 | 0.26 | 2.64 | 57.06 | 17.31 | 15.37 |

| SAMPLE ID | C20:0 | C20:1 | C20:2 | C22:0 | C22:1 | C24:0 | C24:1 | Sats |
|---|---|---|---|---|---|---|---|---|
| FTJ625705 | 0.87 | 0.94 | 0.00 | 0.37 | 0.00 | 0.23 | 0.51 | 10.37 |
| FTJ625740 | 0.80 | 0.93 | 0.00 | 0.31 | 0.00 | 0.21 | 0.44 | 10.12 |
| FTJ625559 | 0.58 | 0.93 | 0.08 | 0.29 | 0.03 | 0.12 | 0.29 | 7.82 |
| FTJ625616 | 0.67 | 0.86 | 0.08 | 0.30 | 0.00 | 0.47 | 0.00 | 9.35 |
| FTJ625632 | 0.70 | 0.89 | 0.06 | 0.24 | 0.00 | 0.28 | 0.11 | 9.19 |
| FTJ625518 | 0.64 | 0.84 | 0.07 | 0.24 | 0.02 | 0.12 | 0.26 | 8.53 |
| FTJ625543 | 0.98 | 1.09 | 0.09 | 0.45 | 0.00 | 0.20 | 0.28 | 8.95 |
| FTJ625717 | 0.82 | 1.06 | 0.08 | 0.39 | 0.00 | 0.20 | 0.41 | 8.38 |
| FTJ625669 | 0.70 | 1.02 | 0.09 | 0.35 | 0.00 | 0.21 | 0.55 | 8.95 |
| FTJ625721 | 0.80 | 1.02 | 0.06 | 0.33 | 0.00 | 0.19 | 0.43 | 8.19 |
| FTJ625644 | 0.82 | 1.05 | 0.08 | 0.43 | 0.00 | 0.00 | 0.46 | 8.46 |
| FTJ625612 | 0.87 | 1.08 | 0.11 | 0.49 | 0.00 | 0.66 | 0.00 | 10.32 |
| FTJ625752 | 0.85 | 0.94 | 0.00 | 0.37 | 0.00 | 0.17 | 0.38 | 9.77 |
| FTJ625720 | 0.82 | 0.88 | 0.06 | 0.29 | 0.00 | 0.23 | 0.65 | 9.51 |
| FTJ625628 | 0.69 | 0.89 | 0.07 | 0.25 | 0.00 | 0.26 | 0.04 | 8.51 |
| FTJ625653 | 1.00 | 1.12 | 0.08 | 0.52 | 0.00 | 0.20 | 0.50 | 9.53 |
| FTJ625542 | 0.74 | 0.86 | 0.08 | 0.27 | 0.00 | 0.15 | 0.22 | 9.33 |
| FTJ625742 | 0.77 | 0.75 | 0.00 | 0.33 | 0.00 | 0.30 | 0.39 | 11.27 |
| FTJ625536 | 0.57 | 0.91 | 0.08 | 0.21 | 0.00 | 0.11 | 0.21 | 7.78 |
| FTJ625519 | 0.86 | 1.08 | 0.08 | 0.39 | 0.04 | 0.22 | 0.31 | 8.24 |
| FTJ625647 | 0.62 | 0.96 | 0.06 | 0.24 | 0.00 | 0.19 | 0.17 | 8.42 |
| FTJ625532 | 0.68 | 0.96 | 0.09 | 0.26 | 0.00 | 0.11 | 0.23 | 7.82 |
| FTJ625530 | 0.59 | 0.80 | 0.08 | 0.26 | 0.03 | 0.12 | 0.25 | 8.46 |
| FTJ625723 | 0.88 | 1.01 | 0.00 | 0.44 | 0.00 | 0.18 | 0.36 | 8.63 |
| FTJ625585 | 0.78 | 0.98 | 0.07 | 0.32 | 0.00 | 0.14 | 0.27 | 8.82 |
| FTJ625626 | 0.89 | 1.10 | 0.09 | 0.48 | 0.00 | 0.56 | 0.36 | 9.30 |
| FTJ625548 | 0.90 | 0.93 | 0.08 | 0.39 | 0.03 | 0.17 | 0.27 | 9.43 |
| FTJ625634 | 0.91 | 1.12 | 0.08 | 0.51 | 0.00 | 0.00 | 0.43 | 9.74 |
| FTJ625664 | 1.11 | 1.04 | 0.06 | 0.47 | 0.00 | 0.21 | 0.64 | 9.93 |
| FTJ625526 | 0.80 | 0.89 | 0.09 | 0.37 | 0.03 | 0.14 | 0.25 | 9.43 |
| FTJ625633 | 0.83 | 1.04 | 0.09 | 0.39 | 0.00 | 0.00 | 0.39 | 8.60 |
| FTJ625726 | 0.68 | 0.89 | 0.00 | 0.28 | 0.00 | 0.16 | 0.36 | 8.85 |
| FTJ625525 | 0.56 | 0.93 | 0.15 | 0.37 | 0.09 | 0.23 | 0.33 | 7.99 |
| FTJ625695 | 0.88 | 1.12 | 0.06 | 0.36 | 0.00 | 0.16 | 0.37 | 8.40 |
| FTJ625649 | 0.91 | 1.09 | 0.07 | 0.40 | 0.00 | 0.14 | 0.38 | 9.09 |
| FTJ625641 | 0.76 | 1.16 | 0.09 | 0.46 | 0.00 | 0.40 | 0.04 | 8.42 |
| FTJ625690 | 0.84 | 1.07 | 0.07 | 0.35 | 0.00 | 0.23 | 0.80 | 8.25 |
| FTJ625535 | 0.75 | 0.95 | 0.09 | 0.44 | 0.00 | 0.33 | 0.52 | 9.22 |
| FTJ625605 | 0.97 | 1.06 | 0.09 | 0.48 | 0.00 | 0.26 | 0.15 | 9.31 |
| FTJ625524 | 0.57 | 1.04 | 0.07 | 0.29 | 0.00 | 0.13 | 0.23 | 7.24 |
| FTJ625718 | 0.83 | 0.90 | 0.05 | 0.30 | 0.00 | 0.25 | 0.52 | 9.63 |
| FTJ625689 | 0.81 | 0.96 | 0.06 | 0.35 | 0.00 | 0.16 | 0.36 | 8.74 |
| FTJ625678 | 0.89 | 0.95 | 0.06 | 0.31 | 0.00 | 0.21 | 0.36 | 9.63 |
| FTJ625688 | 0.64 | 1.08 | 0.06 | 0.31 | 0.00 | 0.25 | 0.67 | 8.13 |
| FTJ625643 | 0.81 | 1.07 | 0.02 | 0.40 | 0.00 | 0.31 | 0.06 | 8.93 |
| FTJ625735 | 0.78 | 0.94 | 0.00 | 0.33 | 0.00 | 0.18 | 0.38 | 9.07 |
| FTJ625703 | 0.76 | 1.13 | 0.06 | 0.37 | 0.00 | 0.23 | 0.38 | 8.28 |
| FTJ625615 | 0.85 | 1.06 | 0.08 | 0.36 | 0.00 | 0.29 | 0.16 | 9.21 |
| FTJ625598 | 0.75 | 0.89 | 0.07 | 0.28 | 0.00 | 0.11 | 0.20 | 8.77 |
| FTJ625696 | 0.86 | 0.94 | 0.06 | 0.38 | 0.00 | 0.16 | 0.37 | 9.06 |
| FTJ625670 | 0.63 | 0.86 | 0.06 | 0.23 | 0.00 | 0.18 | 0.33 | 8.86 |
| FTJ625576 | 0.89 | 1.21 | 0.10 | 0.45 | 0.00 | 0.22 | 0.36 | 8.64 |
| FTJ625732 | 0.83 | 1.06 | 0.00 | 0.39 | 0.00 | 0.16 | 0.55 | 8.38 |
| FTJ625710 | 0.70 | 0.93 | 0.06 | 0.25 | 0.00 | 0.14 | 0.29 | 8.31 |
| FTJ625578 | 0.94 | 1.03 | 0.08 | 0.44 | 0.00 | 0.20 | 0.28 | 9.04 |
| FTJ625668 | 0.86 | 1.28 | 0.07 | 0.36 | 0.00 | 0.26 | 0.39 | 8.04 |
| FTJ625599 | 0.63 | 0.97 | 0.07 | 0.27 | 0.00 | 0.13 | 0.25 | 7.82 |
| FTJ625709 | 1.00 | 1.20 | 0.00 | 0.53 | 0.00 | 0.42 | 0.48 | 9.39 |
| FTJ625520 | 0.53 | 0.76 | 0.07 | 0.19 | 0.00 | 0.14 | 0.24 | 8.30 |
| FTJ625523 | 0.72 | 1.07 | 0.10 | 0.39 | 0.06 | 0.29 | 0.37 | 8.17 |
| FTJ625682 | 0.83 | 1.15 | 0.06 | 0.39 | 0.00 | 0.23 | 0.43 | 8.61 |
| FTJ625749 | 0.74 | 1.17 | 0.08 | 0.28 | 0.00 | 0.19 | 0.36 | 8.10 |
| FTJ625640 | 0.75 | 0.80 | 0.02 | 0.25 | 0.00 | 0.16 | 0.06 | 9.27 |
| FTJ625521 | 0.75 | 1.11 | 0.08 | 0.40 | 0.03 | 0.24 | 0.29 | 8.27 |
| FTJ625636 | 0.74 | 0.99 | 0.07 | 0.27 | 0.00 | 0.25 | 0.07 | 8.32 |
| FTJ625592 | 0.72 | 1.18 | 0.06 | 0.33 | 0.00 | 0.20 | 0.28 | 7.66 |
| FTJ625584 | 0.67 | 0.95 | 0.06 | 0.26 | 0.00 | 0.13 | 0.23 | 7.98 |
| FTJ625645 | 0.87 | 1.20 | 0.00 | 0.35 | 0.00 | 0.00 | 0.44 | 8.29 |
| FTJ625691 | 0.69 | 0.86 | 0.06 | 0.26 | 0.00 | 0.16 | 0.33 | 9.41 |
| FTJ625648 | 0.58 | 0.87 | 0.05 | 0.21 | 0.00 | 0.07 | 0.40 | 7.93 |
| FTJ625753 | 0.87 | 0.98 | 0.00 | 0.36 | 0.00 | 0.34 | 0.39 | 9.85 |
| FTJ625603 | 0.92 | 1.04 | 0.07 | 0.43 | 0.00 | 0.20 | 0.03 | 8.77 |
| FTJ625692 | 0.89 | 1.04 | 0.06 | 0.32 | 0.00 | 0.16 | 0.28 | 9.10 |
| FTJ625727 | 0.68 | 1.00 | 0.00 | 0.31 | 0.00 | 0.15 | 0.33 | 7.86 |
| FTJ625556 | 0.61 | 0.95 | 0.08 | 0.28 | 0.00 | 0.20 | 0.18 | 7.96 |
| FTJ625660 | 0.55 | 0.81 | 0.06 | 0.23 | 0.00 | 0.15 | 0.71 | 8.70 |
| FTJ625575 | 0.78 | 0.93 | 0.06 | 0.33 | 0.00 | 0.19 | 0.25 | 9.42 |
| FTJ625731 | 0.67 | 0.82 | 0.00 | 0.26 | 0.00 | 0.15 | 0.29 | 8.82 |
| FTJ625533 | 0.67 | 1.00 | 0.09 | 0.34 | 0.04 | 0.15 | 0.25 | 8.12 |
| FTJ625725 | 0.71 | 0.96 | 0.00 | 0.27 | 0.00 | 0.18 | 0.30 | 8.86 |
| FTJ625683 | 0.85 | 1.02 | 0.06 | 0.30 | 0.00 | 0.17 | 0.37 | 8.82 |
| FTJ625541 | 0.69 | 1.10 | 0.10 | 0.43 | 0.06 | 0.27 | 0.21 | 8.85 |
| FTJ625586 | 0.13 | 0.79 | 0.00 | 0.24 | 0.00 | 0.13 | 0.34 | 8.31 |
| FTJ625712 | 0.90 | 1.08 | 0.06 | 0.36 | 0.00 | 0.24 | 0.53 | 9.15 |
| FTJ625589 | 0.84 | 0.97 | 0.06 | 0.33 | 0.00 | 0.19 | 0.27 | 9.25 |
| FTJ625666 | 1.07 | 0.99 | 0.06 | 0.42 | 0.00 | 0.23 | 0.33 | 11.11 |
| FTJ625614 | 0.91 | 0.88 | 0.06 | 0.32 | 0.00 | 0.31 | 0.06 | 9.82 |
| FTJ625657 | 0.64 | 1.04 | 0.07 | 0.31 | 0.00 | 0.13 | 0.41 | 7.59 |
| FTJ625734 | 0.96 | 1.10 | 0.00 | 0.64 | 0.00 | 0.37 | 0.75 | 10.03 |
| FTJ625681 | 0.87 | 1.04 | 0.07 | 0.45 | 0.00 | 0.23 | 0.81 | 9.26 |
| FTJ625676 | 0.73 | 1.04 | 0.05 | 0.32 | 0.00 | 0.19 | 0.42 | 8.55 |
| FTJ625639 | 0.77 | 1.12 | 0.06 | 0.32 | 0.00 | 0.30 | 0.04 | 8.30 |
| FTJ625522 | 0.85 | 1.04 | 0.08 | 0.42 | 0.03 | 0.23 | 0.28 | 8.91 |
| FTJ625528 | 0.70 | 0.99 | 0.09 | 0.37 | 0.05 | 0.25 | 0.42 | 8.55 |
| FTJ625568 | 0.61 | 0.94 | 0.09 | 0.23 | 0.00 | 0.09 | 0.17 | 7.92 |
| FTJ625674 | 0.68 | 1.12 | 0.08 | 0.33 | 0.00 | 0.15 | 0.41 | 7.36 |
| FTJ625606 | 0.83 | 1.33 | 0.10 | 0.47 | 0.00 | 0.25 | 0.15 | 8.61 |
| FTJ625604 | 0.66 | 0.94 | 0.06 | 0.25 | 0.00 | 0.19 | 0.15 | 8.03 |
| FTJ625686 | 0.89 | 1.00 | 0.08 | 0.41 | 0.00 | 0.21 | 0.39 | 9.17 |
| FTJ625713 | 0.61 | 0.96 | 0.05 | 0.27 | 0.00 | 0.25 | 0.59 | 8.08 |
| FTJ625693 | 0.89 | 0.97 | 0.07 | 0.39 | 0.00 | 0.18 | 0.34 | 9.12 |
| FTJ625741 | 1.00 | 1.05 | 0.00 | 0.45 | 0.00 | 0.42 | 0.49 | 9.91 |
| FTJ625620 | 0.58 | 0.78 | 0.07 | 0.23 | 0.00 | 0.35 | 0.07 | 8.43 |
| FTJ625537 | 0.64 | 1.10 | 0.08 | 0.29 | 0.00 | 0.17 | 0.22 | 7.76 |
| FTJ625663 | 0.75 | 1.04 | 0.07 | 0.28 | 0.00 | 0.22 | 0.36 | 8.71 |
| FTJ625677 | 0.68 | 0.98 | 0.08 | 0.30 | 0.00 | 0.20 | 0.37 | 7.96 |
| FTJ625527 | 0.68 | 1.02 | 0.07 | 0.33 | 0.04 | 0.19 | 0.21 | 8.22 |
| FTJ625635 | 0.76 | 1.18 | 0.08 | 0.36 | 0.00 | 0.27 | 0.09 | 8.19 |
| FTJ625724 | 0.77 | 0.94 | 0.00 | 0.39 | 0.00 | 0.20 | 0.45 | 9.17 |
| FTJ625722 | 0.72 | 1.10 | 0.00 | 0.42 | 0.00 | 0.16 | 0.32 | 8.23 |
| FTJ625680 | 0.76 | 1.09 | 0.05 | 0.33 | 0.00 | 0.20 | 0.38 | 8.14 |
| FTJ625675 | 0.66 | 0.97 | 0.06 | 0.25 | 0.00 | 0.13 | 0.36 | 8.21 |
| FTJ625698 | 1.01 | 1.13 | 0.07 | 0.44 | 0.00 | 0.26 | 0.71 | 8.51 |
| FTJ625596 | 0.72 | 1.17 | 0.07 | 0.31 | 0.00 | 0.17 | 0.27 | 7.24 |
| FTJ625714 | 0.80 | 1.08 | 0.05 | 0.36 | 0.00 | 0.31 | 0.63 | 8.29 |
| FTJ625569 | 0.79 | 1.14 | 0.08 | 0.38 | 0.03 | 0.17 | 0.21 | 7.76 |
| FTJ625667 | 0.53 | 0.89 | 0.06 | 0.22 | 0.00 | 0.11 | 0.30 | 7.59 |
| FTJ625697 | 0.91 | 1.07 | 0.07 | 0.46 | 0.00 | 0.46 | 1.58 | 9.91 |
| FTJ625748 | 0.99 | 1.06 | 0.00 | 0.43 | 0.00 | 0.23 | 0.30 | 9.09 |
| FTJ625613 | 0.63 | 0.97 | 0.06 | 0.23 | 0.00 | 0.26 | 0.09 | 7.77 |
| FTJ625672 | 0.82 | 1.11 | 0.06 | 0.41 | 0.00 | 0.24 | 0.36 | 8.35 |
| FTJ625595 | 0.59 | 0.88 | 0.06 | 0.24 | 0.00 | 0.11 | 0.19 | 7.70 |
| FTJ625646 | 0.85 | 0.98 | 0.06 | 0.43 | 0.00 | 0.00 | 0.34 | 9.95 |
| FTJ625746 | 0.61 | 1.05 | 0.00 | 0.21 | 0.00 | 0.19 | 0.26 | 8.09 |
| FTJ625706 | 0.88 | 0.97 | 0.07 | 0.43 | 0.00 | 0.28 | 0.38 | 10.62 |
| FTJ625594 | 0.74 | 0.99 | 0.09 | 0.33 | 0.00 | 0.23 | 0.36 | 9.70 |
| FTJ625704 | 0.60 | 1.01 | 0.05 | 0.26 | 0.00 | 0.18 | 0.31 | 7.39 |
| FTJ625684 | 0.80 | 1.10 | 0.06 | 0.37 | 0.00 | 0.21 | 0.39 | 8.38 |
| FTJ625627 | 0.87 | 1.13 | 0.00 | 0.43 | 0.00 | 0.47 | 0.00 | 8.57 |
| FTJ625564 | 0.79 | 1.17 | 0.10 | 0.43 | 0.03 | 0.23 | 0.23 | 8.10 |
| FTJ625652 | 0.94 | 0.97 | 0.06 | 0.35 | 0.00 | 0.13 | 0.31 | 9.83 |
| FTJ625659 | 0.61 | 0.86 | 0.05 | 0.22 | 0.00 | 0.12 | 0.30 | 8.22 |
| FTJ625608 | 0.77 | 1.03 | 0.06 | 0.32 | 0.00 | 0.20 | 0.14 | 8.82 |
| FTJ625621 | 0.60 | 1.05 | 0.07 | 0.30 | 0.00 | 0.29 | 0.04 | 7.44 |
| FTJ625557 | 0.73 | 1.05 | 0.09 | 0.43 | 0.04 | 0.21 | 0.23 | 9.42 |
| FTJ625661 | 0.75 | 1.04 | 0.06 | 0.35 | 0.00 | 0.23 | 0.51 | 8.40 |
| FTJ625551 | 0.58 | 1.06 | 0.09 | 0.34 | 0.05 | 0.18 | 0.20 | 7.39 |
| FTJ625538 | 0.58 | 0.87 | 0.09 | 0.21 | 0.00 | 0.13 | 0.16 | 7.73 |
| FTJ625656 | 0.94 | 1.03 | 0.07 | 0.42 | 0.00 | 0.19 | 0.35 | 9.48 |
| FTJ625618 | 0.77 | 1.00 | 0.07 | 0.34 | 0.00 | 0.41 | 0.00 | 8.64 |
| FTJ625671 | 0.78 | 1.02 | 0.06 | 0.36 | 0.00 | 0.19 | 0.33 | 8.36 |
| FTJ625679 | 0.70 | 0.92 | 0.05 | 0.30 | 0.00 | 0.18 | 0.29 | 8.80 |
| FTJ625562 | 0.52 | 0.94 | 0.09 | 0.20 | 0.00 | 0.13 | 0.20 | 7.91 |
| FTJ625716 | 0.78 | 0.95 | 0.04 | 0.36 | 0.00 | 0.20 | 0.32 | 9.15 |
| FTJ625673 | 0.84 | 0.96 | 0.06 | 0.32 | 0.00 | 0.20 | 0.30 | 8.62 |
| FTJ625655 | 0.80 | 0.97 | 0.06 | 0.31 | 0.00 | 0.15 | 0.43 | 8.70 |
| FTJ625650 | 0.56 | 0.93 | 0.06 | 0.23 | 0.00 | 0.17 | 0.15 | 7.52 |
| FTJ625651 | 0.89 | 0.99 | 0.00 | 0.40 | 0.00 | 0.18 | 0.54 | 9.03 |
| FTJ625567 | 0.65 | 0.94 | 0.05 | 0.27 | 0.02 | 0.14 | 0.15 | 8.02 |
| FTJ625654 | 0.79 | 1.24 | 0.00 | 0.39 | 0.00 | 0.24 | 0.74 | 7.91 |
| FTJ625699 | 0.54 | 0.83 | 0.07 | 0.26 | 0.00 | 0.17 | 0.32 | 8.62 |
| FTJ625719 | 0.71 | 1.04 | 0.00 | 0.26 | 0.00 | 0.18 | 0.35 | 7.86 |
| FTJ625573 | 0.73 | 1.09 | 0.00 | 0.38 | 0.00 | 0.19 | 0.40 | 8.11 |
| FTJ625700 | 0.70 | 1.06 | 0.05 | 0.30 | 0.00 | 0.26 | 0.34 | 8.02 |
| FTJ625685 | 0.80 | 1.08 | 0.06 | 0.37 | 0.00 | 0.25 | 0.34 | 8.52 |
| FTJ625546 | 0.88 | 1.18 | 0.08 | 0.45 | 0.02 | 0.23 | 0.22 | 8.41 |
| FTJ625715 | 0.00 | 1.01 | 0.00 | 0.42 | 0.00 | 0.31 | 0.65 | 8.99 |
| FTJ625750 | 0.63 | 1.01 | 0.00 | 0.24 | 0.00 | 0.12 | 0.23 | 7.30 |
| FTJ625745 | 0.63 | 0.90 | 0.00 | 0.24 | 0.00 | 0.13 | 0.22 | 8.10 |
| FTJ625600 | 0.79 | 1.04 | 0.00 | 0.37 | 0.00 | 0.17 | 0.28 | 8.06 |
| FTJ625658 | 0.78 | 1.08 | 0.00 | 0.33 | 0.00 | 0.23 | 0.88 | 8.10 |
| FTJ625665 | 0.81 | 1.02 | 0.05 | 0.34 | 0.00 | 0.13 | 0.24 | 8.89 |
| FTJ625708 | 0.85 | 1.31 | 0.07 | 0.41 | 0.00 | 0.34 | 0.39 | 8.04 |
| FTJ625743 | 0.72 | 1.02 | 0.06 | 0.33 | 0.00 | 0.23 | 0.31 | 8.08 |
| FTJ625619 | 0.93 | 0.98 | 0.06 | 0.40 | 0.00 | 0.27 | 0.05 | 9.29 |
| FTJ625730 | 0.73 | 0.95 | 0.00 | 0.33 | 0.00 | 0.24 | 0.26 | 8.56 |
| FTJ625597 | 0.77 | 1.14 | 0.07 | 0.37 | 0.00 | 0.20 | 0.27 | 7.68 |
| FTJ625571 | 0.73 | 1.00 | 0.08 | 0.38 | 0.00 | 0.23 | 0.19 | 8.45 |
| FTJ625609 | 0.78 | 1.05 | 0.00 | 0.38 | 0.00 | 0.26 | 0.34 | 8.45 |
| FTJ625588 | 0.14 | 0.95 | 0.06 | 0.30 | 0.00 | 0.18 | 0.20 | 7.37 |
| FTJ625739 | 0.77 | 1.22 | 0.00 | 0.40 | 0.00 | 0.23 | 0.29 | 7.68 |
| FTJ625701 | 0.70 | 1.06 | 0.05 | 0.29 | 0.00 | 0.19 | 0.34 | 7.71 |
| FTJ625602 | 0.72 | 0.97 | 0.07 | 0.35 | 0.00 | 0.14 | 0.24 | 8.37 |
| FTJ625707 | 0.71 | 1.05 | 0.06 | 0.33 | 0.00 | 0.21 | 0.33 | 8.31 |
| FTJ625554 | 0.82 | 1.01 | 0.08 | 0.32 | 0.00 | 0.17 | 0.16 | 8.45 |
| FTJ625610 | 0.96 | 1.18 | 0.05 | 0.44 | 0.00 | 0.26 | 0.06 | 8.84 |
| FTJ625733 | 0.80 | 1.18 | 0.00 | 0.37 | 0.00 | 0.18 | 0.40 | 8.26 |
| FTJ625736 | 0.99 | 1.26 | 0.00 | 0.65 | 0.00 | 0.31 | 1.61 | 9.93 |
| FTJ625694 | 0.63 | 0.86 | 0.00 | 0.27 | 0.00 | 0.17 | 0.41 | 8.79 |
| FTJ625747 | 0.90 | 1.06 | 0.07 | 0.37 | 0.00 | 0.22 | 0.27 | 8.91 |
| FTJ625642 | 0.82 | 0.93 | 0.00 | 0.33 | 0.00 | 0.00 | 0.81 | 9.70 |
| FTJ625558 | 0.58 | 0.76 | 0.07 | 0.26 | 0.00 | 0.22 | 0.32 | 9.32 |
| FTJ625687 | 0.75 | 1.02 | 0.06 | 0.31 | 0.00 | 0.21 | 0.34 | 8.30 |
| FTJ625534 | 0.68 | 0.86 | 0.06 | 0.29 | 0.02 | 0.16 | 0.18 | 8.54 |
| FTJ625611 | 0.83 | 1.01 | 0.00 | 0.44 | 0.00 | 0.51 | 0.84 | 9.77 |
| FTJ625729 | 0.73 | 1.12 | 0.00 | 0.30 | 0.00 | 0.23 | 0.30 | 7.70 |
| FTJ625590 | 0.82 | 1.09 | 0.06 | 0.37 | 0.00 | 0.24 | 0.23 | 8.64 |
| FTJ625662 | 0.91 | 1.18 | 0.06 | 0.44 | 0.00 | 0.28 | 0.34 | 8.80 |
| FTJ625582 | 0.87 | 0.89 | 0.06 | 0.37 | 0.00 | 0.20 | 0.19 | 9.54 |
| FTJ625638 | 0.77 | 1.28 | 0.07 | 0.43 | 0.00 | 0.25 | 0.05 | 7.81 |
| FTJ625702 | 1.05 | 1.15 | 0.06 | 0.49 | 0.00 | 0.30 | 0.32 | 9.10 |
| FTJ625711 | 0.89 | 1.07 | 0.06 | 0.44 | 0.00 | 0.33 | 0.26 | 8.71 |
| FTJ625570 | 0.76 | 1.07 | 0.09 | 0.37 | 0.03 | 0.11 | 0.07 | 8.03 |
| FTJ625637 | 0.69 | 0.89 | 0.06 | 0.26 | 0.00 | 0.20 | 0.05 | 8.26 |
| FTJ625540 | 0.79 | 1.01 | 0.07 | 0.35 | 0.02 | 0.22 | 0.16 | 8.26 |
| FTJ625623 | 0.62 | 0.95 | 0.06 | 0.26 | 0.00 | 0.17 | 0.06 | 7.33 |
| FTJ625566 | 0.49 | 0.77 | 0.07 | 0.20 | 0.01 | 0.09 | 0.20 | 7.40 |
| FTJ625561 | 0.60 | 0.89 | 0.10 | 0.27 | 0.04 | 0.17 | 0.14 | 8.14 |
| FTJ625550 | 0.65 | 0.75 | 0.11 | 0.29 | 0.00 | 0.10 | 0.28 | 8.78 |
| FTJ625552 | 0.69 | 0.89 | 0.14 | 0.37 | 0.04 | 0.36 | 0.95 | 9.32 |
| FTJ625555 | 0.58 | 0.92 | 0.09 | 0.29 | 0.00 | 0.13 | 0.21 | 8.01 |
| FTJ625544 | 0.70 | 0.73 | 0.14 | 0.36 | 0.00 | 0.23 | 0.20 | 10.33 |
| FTJ625572 | 0.52 | 0.77 | 0.05 | 0.20 | 0.01 | 0.14 | 0.16 | 8.12 |
| FTJ625553 | 0.71 | 0.75 | 0.09 | 0.31 | 0.00 | 0.17 | 0.30 | 9.17 |
| FTJ625574 | 0.58 | 0.82 | 0.07 | 0.25 | 0.00 | 0.14 | 0.22 | 9.00 |
| FTJ625593 | 0.48 | 0.73 | 0.06 | 0.20 | 0.00 | 0.11 | 0.17 | 7.85 |
| FTJ625563 | 0.79 | 1.24 | 0.07 | 0.44 | 0.03 | 0.25 | 0.15 | 7.75 |
| FTJ625549 | 0.86 | 1.25 | 0.08 | 0.47 | 0.02 | 0.22 | 0.18 | 8.09 |
| FTJ625624 | 0.95 | 1.42 | 0.07 | 0.54 | 0.00 | 0.28 | 0.00 | 8.19 |
| FTJ625577 | 0.88 | 1.08 | 0.09 | 0.39 | 0.00 | 0.26 | 0.39 | 9.16 |
| FTJ625625 | 0.96 | 1.36 | 0.08 | 0.53 | 0.00 | 0.26 | 0.06 | 8.52 |
| FTJ625631 | 0.94 | 1.38 | 0.08 | 0.53 | 0.00 | 0.00 | 0.29 | 8.01 |
| FTJ625630 | 0.95 | 1.47 | 0.07 | 0.56 | 0.00 | 0.29 | 0.00 | 8.39 |
| FTJ625629 | 0.92 | 1.37 | 0.07 | 0.49 | 0.00 | 0.28 | 0.00 | 8.16 |
| FTJ625583 | 0.90 | 1.36 | 0.07 | 0.50 | 0.00 | 0.29 | 0.25 | 7.94 |
| FTJ625622 | 1.06 | 1.20 | 0.08 | 0.53 | 0.00 | 0.45 | 0.00 | 9.56 |
| FTJ625591 | 1.00 | 1.18 | 0.09 | 0.51 | 0.00 | 0.22 | 0.40 | 8.81 |
| FTJ625601 | 0.96 | 1.22 | 0.09 | 0.50 | 0.00 | 0.17 | 0.37 | 8.57 |
| FTJ625580 | 0.96 | 1.21 | 0.09 | 0.47 | 0.00 | 0.23 | 0.34 | 8.70 |
| FTJ625581 | 0.85 | 1.24 | 0.11 | 0.49 | 0.00 | 0.21 | 0.41 | 8.07 |
| FTJ625587 | 0.21 | 1.21 | 0.09 | 0.57 | 0.00 | 0.26 | 0.40 | 8.52 |
| FTJ625607 | 1.18 | 1.29 | 0.09 | 0.61 | 0.00 | 0.58 | 0.00 | 10.00 |
| FTJ625579 | 0.97 | 1.27 | 0.10 | 0.51 | 0.00 | 0.25 | 0.37 | 8.75 |
| FTJ625617 | 1.08 | 1.28 | 0.09 | 0.56 | 0.00 | 0.43 | 0.00 | 9.31 |
| FTJ625728 | 0.87 | 1.41 | 0.00 | 0.48 | 0.00 | 0.26 | 0.43 | 8.20 |
| FTJ625751 | 1.07 | 1.21 | 0.00 | 0.54 | 0.00 | 0.24 | 0.40 | 9.09 |
| FTJ625737 | 1.04 | 1.29 | 0.09 | 0.52 | 0.00 | 0.29 | 0.41 | 8.86 |
| FTJ625738 | 0.81 | 1.33 | 0.12 | 0.45 | 0.00 | 0.27 | 0.40 | 8.09 |
| FTJ625539 | 0.93 | 1.15 | 0.09 | 0.47 | 0.00 | 0.22 | 0.29 | 8.67 |
| FTJ625565 | 0.97 | 1.15 | 0.08 | 0.47 | 0.03 | 0.22 | 0.29 | 8.90 |
| FTJ625560 | 0.96 | 1.17 | 0.09 | 0.50 | 0.04 | 0.19 | 0.24 | 8.61 |
| FTJ625531 | 1.01 | 1.15 | 0.09 | 0.51 | 0.04 | 0.25 | 0.31 | 9.06 |
| FTJ625529 | 1.00 | 1.20 | 0.09 | 0.50 | 0.05 | 0.24 | 0.31 | 8.78 |
| FTJ625547 | 0.99 | 1.21 | 0.09 | 0.52 | 0.04 | 0.26 | 0.31 | 8.89 |
| FTJ625545 | 0.90 | 1.20 | 0.09 | 0.47 | 0.05 | 0.22 | 0.25 | 8.41 |

1. Seed of Brassica napus, Brassica oleracea, or Brassica juncea, comprising all or part of the genomic sequence of B. napus line rrm1367-003 between SNP markers: C2-p16531874 and C2-p51360247;
wherein said part of the genomic sequence is greater than 200 base pairs.

2. The seed of claim 1 comprising all or part of the genomic sequence between any two SNP markers selected from the group consisting of:
C2-p25019477, C2-p25478505, C2-p25656807, C2-p25913678, C2-p26147167, C2-p26159348, C2-p26207733, C2-p27157822, C2-p27601989, C2-p28031338, C2-p28070964, C2-p28698152, C2-p28806917, C2-p29076828, C2-p29348165, C2-p29383684, 5C00434-p169753, C2-p29474845, C2-p29474845, C2-p29505033, C2-p29505741, C2-p29607300, C2-p29984659, C2-p30062266, C2-p30070472, C2-p30110169, C2-p30154901, C2-p30162991, C2-p30402845, C2-p30431524, C2-p30771286, C2-p30902832, C2-p30942623, C2-p31035160, C2-p31230778, C2-p31354336, C2-p31475220, C2-p31485080, C2-p31502391, C2-p31807771, C2-p31985379, C2-p32008623, C2-p32147720, C2-p32588191, C2-p3353696791, C2-p33633673, C2-p33653822, C2-p33745239, C2-p33761702, C2-p33897506, C2-p33982349, C2-p34550916, C13529254-p142, C2-p34723961, C2-p34766378, C2-p35082231, C2-p35629571, C2-p36261423, C2-p36532052, C2-p36905514, C2-p37181623, C2-p38415038, A02-p21713756 A02-p25181726, and C2-p51360247.

3. Seed of Brassica napus, Brassica oleracea, or Brassica juncea, comprising all or part of the genomic sequence of B. napus line rrm1367-003 between SNP markers: C7-p4690293 and C7-p22870500.

4. The seed of claim 3 comprising all or part of the genomic sequence between any two SNP markers selected from the group consisting of: C7-p4690293, C7-p5039845, C7-p5194981, C7-p7498659, C7-p8599974, C7-p8719053, C7-p8726636, C7-p8726743, C7-p8727745, C7-p8766230, C7-p8824122, C7-p8854349, C7-p8870860, C7-p9307503, C7-p9358459, C7-p9593996, C7-p10040604, C7-p10165832, C7-p10180076, C7-p10180716, C7-p10212158, C7-p10215060, C7-p10215325, C7-p10228536, C7-p10261396, C7-p10262047, C7-p10613314, C7-p10617039, C7-p10720977, C7-p11706153, C7-p11718201, C7-p12072579, C7-p12079142, C7-p12123100, C7-p12123399, C7-p12268682, C7-p12281546, C7-p12300699, C7-p12300699, C7-p12301957, C7-p12356302, C7-p12356455, C7-p12385657, C7-p12387173, C7-p12401233, C7-p12485308, C7-p12508706, C7-p12512146, C7-p12514520, C7-p12565005, C7-p12684624, C7-p12757060, C7-p12984513, C7-p12990275, C7-p12995305, C7-p13029440, C7-p13029555, C7-p13069990, C7-p13070860, C7-p13083371, C7-p13135120, C7-p22861548, C7-p22870500, and C7-p22897297.

5. The seed of claim 3, comprising all or part of the genomic sequence of B. napus rrm1367-003 between SNP markers: C2-p1653187 and C2-p51360247; and/or C7-p4690293 and C7-p22897297, which genomic sequence when introduced B. napus cv. Topas, ATCC deposit PTA-120738, results in an increase in the 18:3 content of the seed oil fraction of seeds produced by the plant into which the fragment has been introduced (e.g., by breeding) relative to B. napus cv. Topas grown under the same or substantially the same conditions.

6. The seed of claim 3, wherein at least one part of the genomic sequence of B. napus rrm1367-003 present in said seed has a length greater than 500 base pairs.

7. The seed of claim 1, wherein the alpha linolenic acid content is greater than 1.4 times higher than a reference strain selected from: B. napus cv. Topas; or B. napus cv. AV-Sapphire, breeders code RO011; wherein said reference strain is grown under the same or substantially the same conditions, and said seed is harvested under the same or substantially the same conditions.

8. The seed of claim 1, wherein the alpha linolenic acid content is greater than 1.4, 1.5, 1.6, 1.7, 1.8, 1.9, 2.0, 2.1, 2.2, or 2.3 times higher than the reference strain B. napus cv. Topas, wherein said reference strain is grown under the same or substantially the same conditions, and said seed is harvested under the same or substantially the same conditions.

9. The seed of claim 1, wherein the alpha linolenic acid content is greater than 1.4, 1.5, or 1.6 times higher than the reference strain B. napus cv. AV-Sapphire, breeders code RO011, wherein said reference strain is grown under the same or substantially the same conditions, and said seed is harvested under the same or substantially the same conditions.

10. The seed of claim 1, wherein the seed has an oil fraction with an 18:3 fatty acid content greater than 7, 8, 9, 10, 11, 12, 13, 14, 15, 16, 17, 18, 19, 20, 21, or 22 percent by weight of the oil fraction, or in a range selected from 7-9, 9-12, 12-15, 15-19, or 19-23 percent by weight of the oil fraction.

11. The seed of claim 1, wherein the seed has an oil fraction with a linolenic acid content in a range selected from 26-24, 24-22, 22-20, 20-16, 19-15, 18-15, or 17-14 percent by weight.

12. The seed of claim 1, wherein the seed has an oil fraction with an oleic acid content less than 69, 68, 66, 64, 62, 61,60, 58, 56, 54, 52, 50, 48, 46, 44, or 42 percent by weight or in a range selected from 69-60, 65-53, 60-50, or 50-41 percent by weight.

13. The seed of claim 1, wherein the seed has an oil fraction with:
a linolenic acid content is greater than 16 percent by weight;
an 18:1 fatty acid content in a range selected from 41-50, 45-55, or 50-60 percent by weight; and
an 18:2 fatty acid in a range selected from 15-20 or 20-24 percent by weight.

14. The seed of claim 1, wherein the seed has an oil fraction with:
a linolenic acid content is greater than 17 percent by weight;
an 18-1 fatty acid content in a range selected from 44-50, 46-55, 45-56, 50-55, or 50-57 percent by weight; and
an 18:2 fatty acid in a range selected from 15-20 or 20-24 percent by weight.

15. The seed of claim 1, having less than 2, 1, 0.5, or 0.1 percent erucic acid by weight of the seed oil fraction.

16. A plant grown from the seed claim 1 or a part thereof, wherein said plant is non-transgenic, transgenic, or transgenic subject to the proviso that the only transgenes present are genes for herbicide resistance.

17. A plant, part thereof according to claim 16 expressing a herbicide tolerance to a herbicide selected from the group consisting of imidazolinone, dicamba, cyclohexanedione, sulfonylurea, glyphosate, glufosinate, phenoxy propionic acid, L-phosphinothricin, triazine and benzonitrile.

18. A plant, part thereof, cell, or protoplast of claim 16, having insect resistance conferred by a gene encoding a Bacillus thuringiensis endotoxin which is expressed in said plant, part thereof, cell, or protoplast.

19. The plant of claim 16, wherein the seed has a meal fraction that contains less than 10, 15, 20, 25, 30, 35, or 40 micromoles of any one or more of 3-butenyl glucosinolate, 4-pentenyl glucosinolate, 2-hydroxy-3 butenyl glucosinolate, and 2-hydroxy-4-pentenyl glucosinolate per gram of dry (air-dry), oil-free solid.

20. Oil from a seed of claim 1, or from a plant, or part thereof, grown from a seed of claim 1, wherein said oil comprises nucleic acids having all or part of the genomic sequence of B. napus line rrm1367-003.

21. Brassica napus=1367-003 deposited as ATCC Accession number PTA-120636, or a progeny thereof having an oil fraction with a linolenic acid content greater than about 16, 17, 19, 20, or 21 percent by weight.

22. A non-transgenic B. napus, B. olereca, or B. juncea plant, or parts thereof, having a low-saturated-fat trait that produces seed having an oil fraction with a linolenic acid content of at least 20 percent by weight.

23. The plant of claim 22, wherein the oil fraction has an erucic acid content of less than 2 percent by weight.

24. A seed or a plant cell from a B. napus, B. olereca, or B. juncea plant whose seed has an oil fraction and a meal fraction, the oil fraction having a linolenic acid content of at least 20 percent by weight and the meal fraction being free of any transgenes other than transgenes conferring herbicide tolerance.

25. A plant produced by the seed of claim 24.

26. The progeny or seed of a plant of claim 25, wherein the seed has an oil fraction with a linolenic acid content of at least 20 percent by weight.


SEQUENCE LISTING (159 sequences)

SEQ ID NO: 1 - DNA, 61 bp, Brassica napus
aaacaaatttattaaccaaatcgtaaaaggrttccagctagaattttctctctaggtttaa

SEQ ID NO: 2 - DNA, 61 bp, Brassica napus
ttgtttagtagatctcaaaagcagatctttrtatagagttggtgagtggcttagtggttcc

SEQ ID NO: 3 - DNA, 61 bp, Brassica napus
gttctaggcgagaaaatgaggtttctgcaarcatacttatcagaaayagacacaagactaa

SEQ ID NO: 4 - DNA, 61 bp, Brassica napus
tgccatagttggagctctagttgtccaagarcccaatttctttttgatatgtgatatctta

SEQ ID NO: 5 - DNA, 61 bp, Brassica napus
ttcggttccggttattttgsccaggcctatmcgaaagtatcgagcttttacatcaatggac

SEQ ID NO: 6 - DNA, 61 bp, Brassica napus
tttttcaaaaataaatgaacccgaatgacartctttwttagacattttttaccactagacc

SEQ ID NO: 7 - DNA, 61 bp, Brassica napus; identical to SEQ ID NO: 6

SEQ ID NO: 8 - DNA, 61 bp, Brassica napus
tggaggaacaggcgttccttatcaaatttaraagaagaaaaaaccctgcactaattcatcc

SEQ ID NO: 9 - DNA, 61 bp, Brassica napus
acatggctagcgatccttggacgtctgcctrcaagagacatgctctcctcttggggcttgt

SEQ ID NO: 10 - DNA, 61 bp, Brassica napus
tcggatttgttaatttagtaatttacatccrgtaaacattttcasagctrgttgactaaaa

SEQ ID NO: 11 - DNA, 61 bp, Brassica napus
ctctacataactgatgctgcagagcaagtamatgccaaaaaaatcrttttgaaaaaaatag

SEQ ID NO: 12 - DNA, 61 bp, Brassica napus
atatgctgttctcgccgtttttgtataactrgatttgggaagttgacaggatcattcggtt

SEQ ID NO: 13 - DNA, 61 bp, Brassica napus
aagatccggctcaatatgtcaaccaaactcmtatcttaccagcaatcttttacacaacatc

SEQ ID NO: 14 - DNA, 61 bp, Brassica napus
aaaccggactggaagatgaaccatctacttrtttggwtcatggttcaatatggttcaactg

SEQ ID NO: 15 - DNA, 61 bp, Brassica napus
tcaattgtgccgttgcagcttgcattgtatrttttaaaagttaattaacacgtcgacctta

SEQ ID NO: 16 - DNA, 61 bp, Brassica napus
taaaataaattgcagggtacaaccaggggarggcgcacggtgtaatgtacgggggcgcgta

SEQ ID NO: 17 - DNA, 61 bp, Brassica napus
cttataaagttataactagatgttttgttcrtatatgcatgtataattcttttatgatamt

SEQ ID NO: 18 - DNA, 61 bp, Brassica napus
ragatacaaaatttgctcaacgttttaaaargaacattaatataggcctaatccaacaaag

SEQ ID NO: 19 - DNA, 61 bp, Brassica napus
taacctgcaaraaaaaaacaaagacttaagmtttggaatcgtcaagaagcaatgaaaacat

SEQ ID NO: 20 - DNA, 61 bp, Brassica napus
aatttttttctaatactgtatctcaaaccamgtatagaaaatagtaaattatataagagtt

SEQ ID NO: 21 - DNA, 61 bp, Brassica napus
tgttttcgtcgttgaactttgtgaatcaatratgacttttaaattcttataagtgtggagg

SEQ ID NO: 22 - DNA, 61 bp, Brassica napus
caacacaatcgtgcgagtttttttatttctrtacttaaatattttagtttatattccctca

SEQ ID NO: 23 - DNA, 61 bp, Brassica napus
tggcttgttaggacatgtaaataagaatgargttaataaacatgggtagttcgtagaagaa

SEQ ID NO: 24 - DNA, 61 bp, Brassica napus
gtgtacaaaagctagaatgaatttttataaraacacagagaacatgttsaacaaaatttca

SEQ ID NO: 25 - DNA, 61 bp, Brassica napus
aagaccgttccatttccataccatggtttamttctctcatscaatatatgmttaatgtaag

SEQ ID NO: 26 - DNA, 61 bp, Brassica napus
tagatagctagttatatataaacaagagtcrttacactaagtattacactataccttaatc

SEQ ID NO: 27 - DNA, 61 bp, Brassica napus
attaaacaagaatgaaagtactgttttgcartgaaagaaaagcycttcttgtctgtttatt

SEQ ID NO: 28 - DNA, 61 bp, Brassica napus
tttagaaaatccggaaatggactattcatcrgtatcaattcgtgggaatttatttgagttt

SEQ ID NO: 29 - DNA, 61 bp, Brassica napus
atcctaaacaaaagaaatcattataataatrtcgaataaaaatacctaaagactaagatta

SEQ ID NO: 30 - DNA, 61 bp, Brassica napus
aagaccggatatmggtcagggtcagggccgrcccagagggaaaaccaccaagccaacgatt

SEQ ID NO: 31 - DNA, 61 bp, Brassica napus
ctatattattaaaatagaaaaacaaatataratttaccctaaaatttataaattatttaca

SEQ ID NO: 32 - DNA, 61 bp, Brassica napus
ggagagactycggagttyttcagatctaaamgaagtttacgacaggaaccggcaccgattg

SEQ ID NO: 33 - DNA, 61 bp, Brassica napus
agatggatattgtctagtaaatctagtatamaaattatggtgagctaggtagtataccttg

SEQ ID NO: 34 - DNA, 61 bp, Brassica napus
tgtggattatagatgacttcaatccgtgttrtcaaagraaaaaraaagatgacttsaatcc

SEQ ID NO: 35 - DNA, 61 bp, Brassica napus
aaaacgaaggttttaccctgaaaatgctgcrggaagccttgctgataccccgcaccttgtg

SEQ ID NO: 36 - DNA, 61 bp, Brassica napus
kaatstacaagmagtgatagactagtgatwracatcttgcaatgatgtgggaagaggttca

SEQ ID NO: 37 - DNA, 61 bp, Brassica napus
tcaatggaaggcmraaaagaatcrtaatccragaacccctatcaggtcctgaccacgagat

SEQ ID NO: 38 - DNA, 61 bp, Brassica napus
gatatcaccaagaatcggagagaatcagtgraaacaaggaaaatgatgaatcagaaagtaa

SEQ ID NO: 39 - DNA, 61 bp, Brassica napus
gttggttcaaaagtagtcgtccacacggacmatgctgcattaaaatatttgatgcaaaaga

SEQ ID NO: 40 - DNA, 61 bp, Brassica napus
ccagacggctttcgcttagccagctctgttmtgcacctcgcaatgcartccataatgctag

SEQ ID NO: 41 - DNA, 61 bp, Brassica napus
agaacaatctgatcgcccgcgactcgaccamccaaaagatacagacgaccccccaaattga

SEQ ID NO: 42 - DNA, 61 bp, Brassica napus
tttcgctgttcaatctcatcagaaacctgcracaaaaatccygataccaggggtctgattg

SEQ ID NO: 43 - DNA, 61 bp, Brassica napus
cgattaaaggttacatacataccttttgggmcgcttataatttaggaagggcttygacraa

SEQ ID NO: 44 - DNA, 61 bp, Brassica napus
tgtttttgaattattagattggaatttgcamtctctaagaattttatttaggcaaatgata

SEQ ID NO: 45 - DNA, 61 bp, Brassica napus
tgccactaccaactccaccaaagtcctttcmmgcatcacatagatcggactcaatctcaag

SEQ ID NO: 46 - DNA, 61 bp, Brassica napus
accccagcaacatcatgaagggcagccttcmactgctrcaacttaatgaagcatacatact

SEQ ID NO: 47 - DNA, 61 bp, Brassica napus
tttgacatcagctcgacctcattcttaaacrctatcattcttttgcccgactttgttgaca

SEQ ID NO: 48 - DNA, 61 bp, Brassica napus
agaatggaacaactactgactccgcaattcrcagaaacgacatggtgctgaaaaaaatagg

SEQ ID NO: 49 - DNA, 61 bp, Brassica napus
atccaatttgaaaaaaaaaatcttcagcaartgataggttgacacatggcgacaaatgccc

SEQ ID NO: 50 - DNA, 61 bp, Brassica napus
aatgaactcatcattgtagatggtgttcttracccaagcaataakatgaagtttgcaacca

SEQ ID NO: 51 - DNA, 61 bp, Brassica napus
cctttaggatatgtgtgaatattaagggtcmacgacttacaagagaaaaaaggtaaaagtt

SEQ ID NO: 52 - DNA, 61 bp, Brassica napus
cttgtcgggagcttgattcttgtctcggagracaagattctcggcttcgagagccccgagc

SEQ ID NO: 53 - DNA, 61 bp, Brassica napus
tttggtgagaaagatatattgtacgcacatmattcgttgatgtaatattttatagttgtaa

SEQ ID NO: 54 - DNA, 61 bp, Brassica napus
agggagcgatttagggcttttatcagatctmttttatacacaagaagaaaaacatttccac

SEQ ID NO: 55 - DNA, 61 bp, Brassica napus
atgctggcattatgaaaatgtttgtaacacmcgtattttttkaaataacttaaataaataa

SEQ ID NO: 56 - DNA, 61 bp, Brassica napus
cgcacgtagtgtctactccttgacaccaacragagattctatcyattgtggataacctgtt

SEQ ID NO: 57 - DNA, 61 bp, Brassica napus
gtatattagatattgtgaaatatggttctammtaagctaaatctcatgtatatcgtatctt

SEQ ID NO: 58 - DNA, 61 bp, Brassica napus
aaatctttccactgattttccatcaatggtmtaaactcatgcatcttagctatcacatttg

SEQ ID NO: 59 - DNA, 61 bp, Brassica napus
attgattrggccgcctaattggttaaatggrccgcttgattggcgtccgattttgttttgc

SEQ ID NO: 60 - DNA, 61 bp, Brassica napus
gaagctgtaaatttycttgggatgaccgagmgcattggtatcgtccgacaaagcatgagtt

SEQ ID NO: 61 - DNA, 61 bp, Brassica napus
ataagatgtgcttttgtaagataatattaamttttgawttttggctttattattttgttaa

SEQ ID NO: 62 - DNA, 61 bp, Brassica napus
tcaatattaggtattgcaaaaactcctagcmaattaacmggtttatctcacttgactaatt

SEQ ID NO: 63 - DNA, 61 bp, Brassica napus
tattggatagctcagaattgattaaagccaraagaagagaaggaagtattggggccawgya

SEQ ID NO: 64 - DNA, 61 bp, Brassica napus
gtaattttagatctgaaaatctgaattcgtmgaaaatgtagaaatagttttaaaattgaaa

SEQ ID NO: 65 - DNA, 61 bp, Brassica napus
atgcttgccaattttattaatactatatacrattttaaaataattcaatacgtacattctt

SEQ ID NO: 66 - DNA, 61 bp, Brassica napus
tagctaacgtttaagcaaatttttgtcaacraaaaatattccttggaggtcaaaatctagt

SEQ ID NO: 67 - DNA, 61 bp, Brassica napus
gtcattcactaataacacatgtttattgtaraagaagaaagaaacgacgaagaamaaaaag

SEQ ID NO: 68 - DNA, 61 bp, Brassica napus
attatcactagaataccacaacttggttacratatggatcttgagtttgcttctttactkc

SEQ ID NO: 69 - DNA, 61 bp, Brassica napus
tgttttcttgtagtgacagtaccgtgttacmgccctaccaaaccggaaggaaactccaaga

SEQ ID NO: 70 - DNA, 61 bp, Brassica napus
tttcgaagagagcccacgaawcaaacaaacmaaaaaagctcaaataaaaagatgccacatg

SEQ ID NO: 71 - DNA, 61 bp, Brassica napus
acactcaggtcgacattaatgtcatctcgarttcgggatggcccttcttctttcstagacc

SEQ ID NO: 72 - DNA, 61 bp, Brassica napus
ggtttcatgttcagaaccaactgtaacatgmgagaaaatatttgatatagaaagacaaaac

SEQ ID NO: 73 - DNA, 61 bp, Brassica napus
tggttcccacagtaaaggagatcatgcaggrtcctcgccggagatcatgctgtcgttacar

SEQ ID NO: 74 - DNA, 61 bp, Brassica napus
caatattttataagggccataaaacttcgrmgccagcttgtaattagatctcatcacagct

SEQ ID NO: 75 - DNA, 61 bp, Brassica napus
tagctataaatctagaaacctgtagaaactragttttacagatttactgtattctacggat

SEQ ID NO: 76 - DNA, 61 bp, Brassica napus
gcaaaaacttccaaattttgaatgaatagtrggagactgaatrttgattccacatttccat

SEQ ID NO: 77 - DNA, 61 bp, Brassica napus
ttgtgcctttaagttttcgggaatttttcaratataacctatatattcatttgaaacataa

SEQ ID NO: 78 - DNA, 61 bp, Brassica napus
aaagaagacatyaataacaaaaaaaactatrtaaggaagcgaggactgaagagaaagcgaa

SEQ ID NO: 79 - DNA, 61 bp, Brassica napus
atccgggacatcgaatattcgagtggctacrgatcgaatcggatcggataattgattattc

SEQ ID NO: 80 - DNA, 61 bp, Brassica napus
tggatgatacttgtcaaacgtgtggagaagmggtcgaaacgctgaaccatatgctgtttca

SEQ ID NO: 81 - DNA, 61 bp, Brassica napus
ataaaaccgcagtgataacaactttaattcmaaaagctccaaatgacagagatatggatgg

SEQ ID NO: 82 - DNA, 61 bp, Brassica napus
tactaaaattcaattggttaaaaattgtggraaatagtcaattgcataaactaatgcattg

SEQ ID NO: 83 - DNA, 61 bp, Brassica napus
ctgaaattctatgtaatctaatggactgatmtgagatactctgtatgttggtggtctttrt

SEQ ID NO: 84 - DNA, 61 bp, Brassica napus
cataagcttagcttgtgtgtacttaagaaaraactcaagaaatcaatagtttatagtttat

SEQ ID NO: 85 - DNA, 61 bp, Brassica napus
tgaagaaatctacatggagcagccagagggrttcattgttaaaggcaaggaagactgggtg

SEQ ID NO: 86 - DNA, 61 bp, Brassica napus
atcaagaaaattacaaattcacgtagatgaratatacagtaggttaaacatataacaacca

SEQ ID NO: 87 - DNA, 61 bp, Brassica napus
atcagtatatacactcacggttccgcgagcmgstctttttgcctcaggtttattggctgac

SEQ ID NO: 88 - DNA, 61 bp, Brassica napus
gaaatgttgcgaatatatgcttaaaagtaargttatatttctaatatgcagttcgaaatag

SEQ ID NO: 89 - DNA, 61 bp, Brassica napus
aacagaatggcaccaggtgtgttggatctcrcactataggaagcacgaaagatctgtrtct

SEQ ID NO: 90 - DNA, 61 bp, Brassica napus
aggttgaatttgatcgttataaaagatctcrtgtgcaagtttaacaacatcaccatcgttm

SEQ ID NO: 91 - DNA, 61 bp, Brassica napus
gaaagaacagttggccgaagcattgactaarccactagaacgatcaagagttcaggagctt

SEQ ID NO: 92 - DNA, 61 bp, Brassica napus
tgtacttcttatagctttaacaaccgatccrgacagtacaatgcaaagtgacctagtttgg

SEQ ID NO: 93 - DNA, 61 bp, Brassica napus
ggagattccaattaaggccaaaactgagtcrgtagaatggttcttagctcaattagtggag

SEQ ID NO: 94 - DNA, 61 bp, Brassica napus
aactctaaatatgcaatccgaagaattaaarcaggagatggaagggttctcacagacctga

SEQ ID NO: 95 - DNA, 61 bp, Brassica napus
gcccaaatatcataaagaaacacacgaaacrctctccgtttttggttttgatcccagacag

SEQ ID NO: 96 - DNA, 61 bp, Brassica napus
agtgatgatccaaaacaattataaaaattaratggcagcttctcagaagaactaagctaac

SEQ ID NO: 97 - DNA, 61 bp, Brassica napus
aaaaatccaattaacaatataaatgtctggmtaatatttcagttagcsraccactaactta

SEQ ID NO: 98 - DNA, 61 bp, Brassica napus
ggcatttgattgagagagatgagcttcatcrctgatgagaaccgagtatgtcgttgaagtc

SEQ ID NO: 99 - DNA, 61 bp, Brassica napus
attataatacttcttttgtcttcctaccacmtttattatttcttccattgaaaagcatgcc

SEQ ID NO: 100 - DNA, 61 bp, Brassica napus
aggaacaaaatggaagttgaagaacacaaaratgggaagaaaaagagaatctaagattaat

SEQ ID NO: 101 - DNA, 61 bp, Brassica napus
ggggagatttacacctacctaaagggtctgmaacctctagttccatgggmaaaagttgtgt

SEQ ID NO: 102 - DNA, 61 bp, Brassica napus
aagtaggtgagccattggacatggtgaatargagagagaggagaagaaacagaggatgawg

SEQ ID NO: 103 - DNA, 61 bp, Brassica napus
ttgacgattccacatgaggtagttggtagargtcagcttcttgaygttggacatgttcaca

SEQ ID NO: 104 - DNA, 61 bp, Brassica napus
actggtttgcctataacacatactggttctrcacttcttcctactcctcatcgttmtctta

SEQ ID NO: 105 - DNA, 61 bp, Brassica napus
gaacacaaatgttgggtccggagctgcgggrggcttagatttacatctcggttgtgtagtt

SEQ ID NO: 106 - DNA, 61 bp, Brassica napus
aaggaatataactgtctaaatagaatatatmactaatgatacagttattcctaccagtgta

SEQ ID NO: 107 - DNA, 61 bp, Brassica napus
actcacaatgcctgtatggaaactaaagaawgagtttcagttgtgttgttctttcagtatc

SEQ ID NO: 108 - DNA, 61 bp, Brassica napus
gatcgagaacatccgcttcttgcctgatcmrgagttcctttgcgccgatctgcgagcaatg

SEQ ID NO: 109 - DNA, 61 bp, Brassica napus
agttcattgyccccttcatgactcgataaawctcttttagaaaacattattagaaaacatt

SEQ ID NO: 110 - DNA, 61 bp, Brassica napus
agaaatctgttttkcttkattkgcaaacrargctagaaagggagatttaagaagttggtga

SEQ ID NO: 111 - DNA, 61 bp, Brassica napus
ggctttgcccagctcattactcttgcaaaamcccttaatgagagtattataggtaacgata

SEQ ID NO: 112 - DNA, 61 bp, Brassica napus
attcagatcacccgattamaaagagagacgmgagcaataagagagataaagagatatcaag

SEQ ID NO: 113 - DNA, 61 bp, Brassica napus
tcatttaagatgttattaacttgacattttmaaatttaagatatctatctaatattttttt

SEQ ID NO: 114 - DNA, 61 bp, Brassica napus
aatttataaaataacagcaaatcttaaaaamtttaatatraaaaatraattatagatatam

SEQ ID NO: 115 - DNA, 61 bp, Brassica napus
tcaaagtcatcttgaaatcgaggattcaccrgcgtagaactcgtcgacatcgtcgcgttgc

SEQ ID NO: 116 - DNA, 61 bp, Brassica napus
tcgggtcctgtccggtgctgtgcctaaatcmaaaggacaccgaggaacaacaccgtacacc

SEQ ID NO: 117 - DNA, 61 bp, Brassica napus
gccatcattgtcaagttcgtcgtcctatccrtaagaatmatwgactggatcctcgtcgttt

SEQ ID NO: 118 - DNA, 61 bp, Brassica napus
cgtgagcgagatgattgattcccccacgtcraagttttctgtagttgttcgaacgtagcag

SEQ ID NO: 119 - DNA, 61 bp, Brassica napus
aaatttatattggtcatttttwtcctatgtrctatttttatgataattttttttagggtta

SEQ ID NO: 120 - DNA, 61 bp, Brassica napus
tcatccacttcctccacagctttcttctccrcaacttccttctcaacctgctcggctgctg

SEQ ID NO: 121 - DNA, 61 bp, Brassica napus
gtaatgcctaaagaaaatcatatataaaatmatgtttctttgatcaaaaagaaaaaaatca

SEQ ID NO: 122 - DNA, 61 bp, Brassica napus
taaaatctccaaaatttattgctctagctargctacaaagtgttgtggccaaacattgatg

SEQ ID NO: 123 - DNA, 61 bp, Brassica napus
ataaccagaaacatagccagacaatgaaagratatgtgatctttctcsaagatcaagtgta

SEQ ID NO: 124 - DNA, 61 bp, Brassica napus
cttgttttttcgttaattggtgtaatatttrtgtttcatataattttattttcagtatttt

SEQ ID NO: 125 - DNA, 61 bp, Brassica napus
aaatayctctaaaaagtcagatattcgattmttggccacccctaggtaccttatcaaagct

SEQ ID NO: 126 - DNA, 61 bp, Brassica napus
tctctttgattatttttaaccatattgactrtagaaactaaaatacacatgtcagtttcar

SEQ ID NO: 127 - DNA, 61 bp, Brassica napus
tctgttatatggtttgagaattttcaaaatmtactattattggtctaagaacttataaaat

SEQ ID NO: 128 - DNA, 61 bp, Brassica napus
cacrtgactatatatgtgaccgraaaaaaartgaaaatcttttttaaaaaaggattgatca

SEQ ID NO: 129 - DNA, 61 bp, Brassica napus
tcatgtttawtaatgtttctagctaatccgrgaagacattaagaatttttctgagagagga

SEQ ID NO: 130 - DNA, 61 bp, Brassica napus
aataagtcaatatttagaaggggatacttcmagagaagtttgytgtagagtagacttccct

SEQ ID NO: 131 - DNA, 61 bp, Brassica napus
agagacatggtggaggagaaaatcatctttmaaacataaaaaactttaaaatatacaraat

SEQ ID NO: 132 - DNA, 61 bp, Brassica napus
agtttaattcattttgagtgaaaaatgaggrtaatcattggaaatgctctaatgtaagcta

SEQ ID NO: 133 - DNA, 61 bp, Brassica napus
ttaaaagtagtcaaaatactcaaaacacttraaatatccaaaatatttacggattctctay

SEQ ID NO: 134 - DNA, 61 bp, Brassica napus
attcccatcatctaacagttttttcaacagmaacracatataaatgctcatataatatttt

SEQ ID NO: 135 - DNA, 61 bp, Brassica napus
gatttattagatctcatattttagccatacrgctcactggcttctgcrtttcattcccctc

SEQ ID NO: 136 - DNA, 61 bp, Brassica napus
aattttcataagactataggtaacattatamgaaacattcattcgtataactgaaacatca

SEQ ID NO: 137 - DNA, 61 bp, Brassica napus
tattgttttttataaaatattacattgagtmggagtttcaatgacttacgaagattaacaa

SEQ ID NO: 138 - DNA, 61 bp, Brassica napus
ttaaaggaaaaatataatgtataatccacamgtagcgaagaccaaaaacctaatatagtga

SEQ ID NO: 139 - DNA, 61 bp, Brassica napus
agaggaggggagcaacactagcaaatggtcrttggttttgccagcaaaaatagggagattt

SEQ ID NO: 140 - DNA, 61 bp, Brassica napus
ctagtgcttcccagaccgtgtaagagtatgramatgcaaaaaacaagtgatctctggtttc

SEQ ID NO: 141 - DNA, 61 bp, Brassica napus
aattgtaacaaagctaagggtctgattgttrcggattttcgctttakgctttcgctttagk

SEQ ID NO: 142 - DNA, 61 bp, Brassica napus
agttaggttatttgctagtgtatcaacgaammatttaatccaatagaacctaggatactca

SEQ ID NO: 143 - DNA, 61 bp, Brassica napus
aaatataggagattggaaatccttctgaacrtgtttttcttgtgccatttgatctcatacc

SEQ ID NO: 144 - DNA, 61 bp, Brassica napus
caaattcagaacaatacagactaagctagcrgtgccaagattaaacygttacaatgtatat

SEQ ID NO: 145 - DNA, 61 bp, Brassica napus
tattataacttggctaagttatgatcttacmaatttagcagtgtgggtatatgcttctctc

SEQ ID NO: 146 - DNA, 61 bp, Brassica napus
ttgatatctatcggccaaatatgctttcagrccatcatatatgattattaatggcatcaag

SEQ ID NO: 147 - DNA, 61 bp, Brassica napus
agggtataggagtccagcatatttggtgtgmattagcatcaatatggtgacttagtatggc

SEQ ID NO: 148 - DNA, 61 bp, Brassica napus
tttagtttgcatgtaaaatgtcaaactgacrctctttgtgcmayagagakaatttaatacg

SEQ ID NO: 149 - DNA, 61 bp, Brassica napus
cacatcgcacggactagaatacagaggacgrggaggtcaaggcagagccggccygtatact

SEQ ID NO: 150 - DNA, 61 bp, Brassica napus
tgcaactgcaactctttagcttttgttgaargtttaaaggtttttctcgaaatcatttttt

SEQ ID NO: 151 - DNA, 61 bp, Brassica napus
ctggaaaagcatctataacaatgtcatgaamcatctcatgatctacyatytgctcttcatg

SEQ ID NO: 152 - DNA, 61 bp, Brassica napus
ttaaaattaccattactactaataagcttcrttcacaccrataaccttytcmatgttgaaa

SEQ ID NO: 153 - DNA, 61 bp, Brassica napus
catagagtgaagacttggccacatttattcrgagcscagacatacaagcaaaccggttcat

SEQ ID NO: 154 - DNA, 61 bp, Brassica napus
tggcgctggcaccacctaatcctgaaagcgraactgatatatttttgtggaaacatgggga

SEQ ID NO: 155 - DNA, 61 bp, Brassica napus
caatttttgttgatsggcaaaaaaacttgcmgaattccgtaatgttaccaatcgggcctaa

SEQ ID NO: 156 - DNA, 61 bp, Brassica napus
cgggaaatatattacatcttatctatcaaarcagaagtcatgatttttttatgtgtgttat

SEQ ID NO: 157 - DNA, 61 bp, Brassica napus
acctcgtgaggacgatctaacataaaatctrtgaactggtraatctctcaggactatgttc

SEQ ID NO: 158 - DNA, 61 bp, Brassica napus
ttttaagttgttgttttcttatgtctttttrtcckattctttttttgttcatattgaatgt

SEQ ID NO: 159 - DNA, 61 bp, Brassica napus
gtatgactattttgtatgattttttcttgarttcttgtacaagacctgcttttcgaccctg